(19) United States
(12) Patent Application Publication
(10) Pub. No.: US 2008/0207528 A1
(43) Pub. Date: Aug. 28, 2008

(54) HCV PROTEASE INHIBITORS

(76) Inventors: Syaulan Yang, Ban-Chyao (TW); Kuang-Yuan Lee, Hsinchu City (TW); Rong-Juinn Chen, Puzih City (TW); Pin Lo, Sanchong City (TW); Shao-Ying Liao, Taipei City (TW); Jen-Dar Wu, Taipei City (TW); Chi-Hsin Richard King, Holladay, UT (US)

Correspondence Address: OCCHIUTI ROHLICEK & TSAO, LLP, 10 FAWCETT STREET, CAMBRIDGE, MA, 02138, US

(21) Appl. No.: 12/023,345

(22) Filed: Jan. 31, 2008

Related U.S. Application Data
(60) Provisional application No. 60/887,741, filed on Feb. 1, 2007.

(51) Int. Cl. A61K 38/06; C07D 215/58; A61K 38/05; A61P 31/12; C07K 5/08

(52) U.S. Cl. 514/18; 546/153; 514/19; 530/331

(57) ABSTRACT

This invention relates to the compounds of formula (I) shown below. Each variable in formula (I) is defined in the specification. These compounds can be used to treat hepatitis C virus infection.

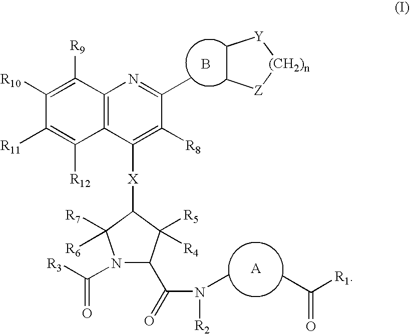

### CROSS REFERENCE

Pursuant to 35 U.S.C. § 119(e), this application claims priority to U.S. Provisional Application 60/887,741 filed on Feb. 1, 2007. The contents of the provisional application are incorporated by reference.

### BACKGROUND

Hepatitis C virus (HCV) is a (+)-sense single-stranded RNA virus that has been implicated as the major causative agent for most cases of non-A, non-B hepatitis. Infection by HCV is a compelling human health problem. See, e.g., WO 89/04669; Alberti et al., J. Hepatology, 31 (Suppl. 1), 17-24 (1999); Alter, J. Hepatology, 31 (Suppl. 1), 88-91 (1999); and Lavanchy, J. Viral Hepatitis, 6, 35-47 (1999).

A HCV protease necessary for viral replication contains about 3000 amino acids. It includes a nucleocapsid protein (C), envelope proteins (E1 and E2), and several non-structural proteins (NS1, NS2, NS3, NS4a, NS5a, and NS5b).

NS3 protein possesses serine protease activity and is considered essential for viral replication and infectivity. The essentiality of the NS3 protease was inferred from the fact that mutations in the yellow fever virus NS3 protease decreased viral infectivity. See, e.g., Chamber et al., Proc. Natl. Acad. Sci. USA 87, 8898-8902 (1990). It was also demonstrated that mutations at the active site of the HCV NS3 protease completely inhibited the HCV infection in chimpanzee model. See, e.g., Rice et al., J. Virol. 74 (4) 2046-51 (2000). Further, the HCV NS3 serine protease was found to facilitate proteolysis at the NS3/NS4a, NS4a/NS4b, NS4b/NS5a, NS5a/NS5b junctions and was thus responsible for generating four viral proteins during viral replication. See, e.g., US 2003/0207861. Consequently, the HCV NS3 serine protease enzyme is an attractive target in treating HCV infection. Potential NS3 HCV protease inhibitors can be found in WO 02/18369, WO 00/09558, WO 00/09543, WO 99/64442, WO 99/07733, WO 99/07734, WO 99/50230, WO 98/46630, WO 98/17679, WO 97/43310, U.S. Pat. No. 5,990,276, Dunsdon et al., Biorg. Med. Chem. Lett. 10, 1571-1579 (2000); Llinas-Brunet et al., Biorg. Med. Chem. Lett. 10, 2267-2270 (2000); and S. LaPlante et al., Biorg. Med. Chem. Lett. 10, 2271-2274 (2000).

Due to lack of immunity or remission associated with HCV infection, hepatitis caused by HCV infection is more difficult to treat comparing to other forms of hepatitis. The only anti-HCV therapies currently available are interferon-α, interferon-α/ribavirin combination, and pegylated interferon-α. However, sustained response rates for interferon-α or interferon-α/ribavirin combination were found to be <50% and patients suffer greatly from side effects of these therapeutic agents. See, e.g., Walker, DDT, 4, 518-529 (1999); Weiland, FEMS Microbial. Rev., 14, 279-288 (1994); and WO 02/18369. Thus, there remains a need for developing more effective and better-tolerated therapeutic drugs.

### SUMMARY

This invention is based on the unexpected discovery that certain proline analogues are effective in treating hepatitis C virus (HCV) infection by inhibiting hepatitis C viral proteases.

In one aspect, the invention features a compound of formula (I):

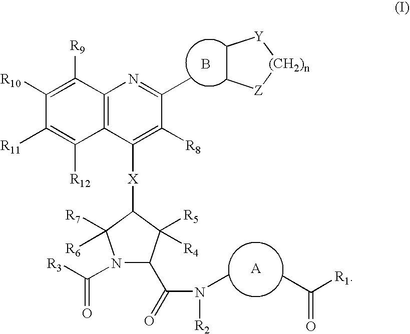

In formula (I), A is C₃-C₅ cycloalkylene, C₃-C₅ cycloalkenylene, or C₇-C₂₀ alkylarylene; B is aryl or heteroaryl; X is O, OCH₂, CH₂O, OC(O), CO(O), C(O)NH, or NHC(O); each of Y and Z, independently, is N(Rₐ₁), O, or CH₂; in which Rₐ₁ is H, C₁-C₁₀ alkyl, C₃-C₂₀ cycloalkyl, C₁-C₂₀ heterocycloalkyl, aryl, or heteroaryl; n is 1 or 2; R₁ is O(R_{b1}), NR_{b1}R_{b2}, NH—O(R_{b1}), NH—C(O)—R_{b1}, NH—C(O)—NR_{b1}R_{b2}, NH—NH—C(O)—R_{b1}, NH—C(O)—NH—S(O)₂—R_{b1}, NH—C(O)—COOR_{b1}, C(O)—NR_{b1}R_{b2}, NH—(R_{b3})—(R_{b4})—S(O)₂—R_{b1}, NH—(R_{b3})—(R_{b4})—S(O)₂—NR_{b1}R_{b2}, or NH—(R_{b3})—(R_{b4})—COO—R_{b1}; in which each of R_{b1} and R_{b2}, independently, is H, C₁-C₁₀ alkyl, C₃-C₂₀ cycloalkyl, C₁-C₂₀ heterocycloalkyl, aryl, or heteroaryl, and each of R_{b3} and R_{b4}, independently, is C₁-C₁₀ alkylene, C₃-C₂₀ cycloalkylene, C₁-C₂₀ heterocycloalkylene, arylene, heteroarylene, or deleted; and each of R₂, R₃, R₄, R₅, R₆, R₇, R₈, R₉, R₁₀, R₁₁, and R₁₂, independently is H, halo, OR_{c1}, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl, in which R_{c1} is H, C₁-C₁₀ alkyl, C₃-C₂₀ cycloalkyl, C₁-C₂₀ heterocycloalkyl, aryl, or heteroaryl.

Referring to formula (I), a subset of the compounds described above are those in which X is O. In these compounds, A can be 1,1-cyclobutylene, 1,1-cyclopentylene, 1,1-cyclopropylene optional substituted with C₂-C₁₀ alkenyl, 1,1-cyclopentenylene optionally substituted with C₂-C₁₀ alkenyl, or

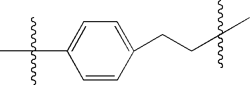

optionally substituted with C₁-C₁₀ alkyl or hydroxyl; B can be phenyl, pyridyl, or thiazole; R₁ can be O(R_{b1}), NH—C(O)—NR_{b1}R_{b2}, NH—NH—C(O)—R_{b1}, NH—C(O)—NH—S(O)₂—R_{b1}, NH—C(O)—COOR_{b1}, NH—(R_{b3})—(R_{b4})—S(O)₂—R_{b1}, NH—(R_{b3})—(R_{b4})—S(O)₂—NR_{b1}R_{b2}, or NH—(R_{b3})—(R_{b4})—COO—R_{b1}; and R₃ can be aryl optionally fused with C₁-C₂₀ heterocycloalkyl, or C₁-C₁₀ alkyl optional substituted with NH—COOR or C₁-C₂₀ heterocycloalkyl, in which R is H, C₁-C₁₀ alkyl, C₃-C₂₀ cycloalkyl, C₁-C₂₀ heterocycloalkyl, aryl, or heteroaryl. For example, R₁ can be OH, NH—S(O)₂—C₁-C₁₀ alkyl, NH—S(O)₂-phenyl, NH—S(O)₂-cyclopropyl, NH—S(O)₂—NH—C₁-C₁₀ alkyl, NH—S(O)₂—NH-phenyl, NH—S(O)₂—NH-cyclopropyl, NH—C(O)—NH-phenyl, NH—NH—C(O)-thienyl, NH—C(O)—NH—S(O)₂-(4-methylphenyl), NH—C(O)—NH-thienyl, NH—C(O)—NH—S(O)₂-phenyl, NH—CH(OH)—COO-ethyl, or NH—C(O)—COO-ethyl; and R₃ can be benzo[1,3]dioxolyl, or isobutyl substituted with NH—COO-t-butyl, NH—COO-cyclopentyl, 3-cyclopentylimidazolidinonyl, or 3-t-butylimidazolidinonyl.

Another subset of the compounds described above are those in which X is OCH₂, CH₂O, OC(O), CO(O), C(O)NH, or NHC(O). In these compounds, B can be phenyl; A can be 1,1-cyclobutylene or 1,1-cyclopropylene optional substituted with C₂-C₁₀ alkenyl; R₁ can be O(R_{b1}) or NH—(R_{b3})—(R_{b4})—S(O)₂—R_{b1}; and R₃ can be C₁-C₁₀ alkyl optional substituted with NH—COOR, in which R is H, C₁-C₁₀ alkyl, C₃-C₂₀ cycloalkyl, C₁-C₂₀ heterocycloalkyl, aryl, or heteroaryl. For example, R₁ can be OH, NH—S(O)₂-phenyl, or NH—S(O)₂-cyclopropyl; and R₃ can be isobutyl substituted with NH—COO-t-butyl or NH—COO-cyclopentyl.

Still another subset of the compounds described above are those in which B is phenyl, pyridinyl, or thiazole; n is 1 or 2; and each of Y and Z is O; each of Y and Z is CH; or Y is NH or NC₁-C₁₀ alkyl and Z is CH or O.

Yet another subset of the compounds described above are those in which R₁ is NH—S(O)₂-cyclopropyl.

The term “alkyl” refers to a saturated, linear or branched hydrocarbon moiety, such as —CH₃ or —CH(CH₃)₂. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as a cyclopropyl. The term “cycloalkylene” refers to a saturated, cyclic, divalent hydrocarbon moiety, such as 1,1-cyclopropylene. The term “cycloalkenyl” refers to a non-aromatic, cyclic hydrocarbon moiety that contains at least one ring double bond, such as cyclohexenyl. The term “cycloalkenylene” refers to a non-aromatic, cyclic, divalent hydrocarbon moiety that contains at least one ring double bond, such as 1,1-cyclopentenylene. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), naphthyl, pyrenyl, anthryl, and phenanthryl. The term “heteroaryl” refers to a moiety having one or more aromatic rings that contain at least one ring heteroatom (e.g., N, O, or S). Examples of heteroaryl moieties include furyl, fluorenyl, pyrrolyl, thienyl, oxazolyl, imidazolyl, thiazolyl, pyridyl, pyrimidinyl, quinazolinyl, quinolyl, isoquinolyl, and indolyl. The term “alkylarylene” refers to a divalent hydrocarbon moiety containing an aryl group substituted with an alkyl group in which one electron is located on the aryl group and the other electron is located on the alkyl group, such as

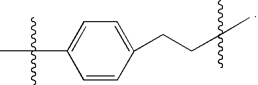

Alkyl, alkenyl, alkynyl, cycloalkyl, cycloalkylene, cycloalkenyl, cycloalkenylene, heterocycloalkyl, heterocycloalkenyl, aryl, heteroaryl, and alkylarylene mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

In another aspect, this invention features a method for treating HCV infection. The method includes administering to a subject in need thereof an effective amount of one or more compounds of formula (I) shown above. The term “treating” or “treatment” refers to administering one or more compounds of formula (I) to a subject, who has a HCV infection, a symptom of it, or a predisposition toward it, with the purpose to confer a therapeutic effect, e.g., to cure, relieve, alter, affect, ameliorate, or prevent the HCV infection, the symptom of it, or the predisposition toward it.

In addition, this invention encompasses a pharmaceutical composition that contains an effective amount of at least one of the compounds of formula (I) and a pharmaceutically acceptable carrier. The composition can further include a second antiviral compound, such as ribavirin or interferon. Examples of interferon include α-interferon or pegylated interferon. The term “pegylated interferon” mentioned herein refers to an interferon that is modified with a polyethylene glycol moiety.

The compounds of formula (I) described above include the compounds themselves, as well as their salts, prodrugs, and solvates, if applicable. A salt, for example, can be formed between an anion and a positively charged group (e.g., amino) on a compound of formula (I). Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a compound of formula (I). Suitable cations include sodium ion, potassium ion, magnesium ion, calcium ion, and an ammonium cation such as tetramethylammonium ion. The compounds of formula (I) also include those salts containing quaternary nitrogen atoms. Examples of prodrugs include esters and other pharmaceutically acceptable derivatives, which, upon administration to a subject, are capable of providing active compounds of formula (I). A solvate refers to a complex formed between an active compound of formula (I) and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the compounds of formula (I) described above for use in treating a HCV infection, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

Shown below are 80 exemplary compounds of this invention.

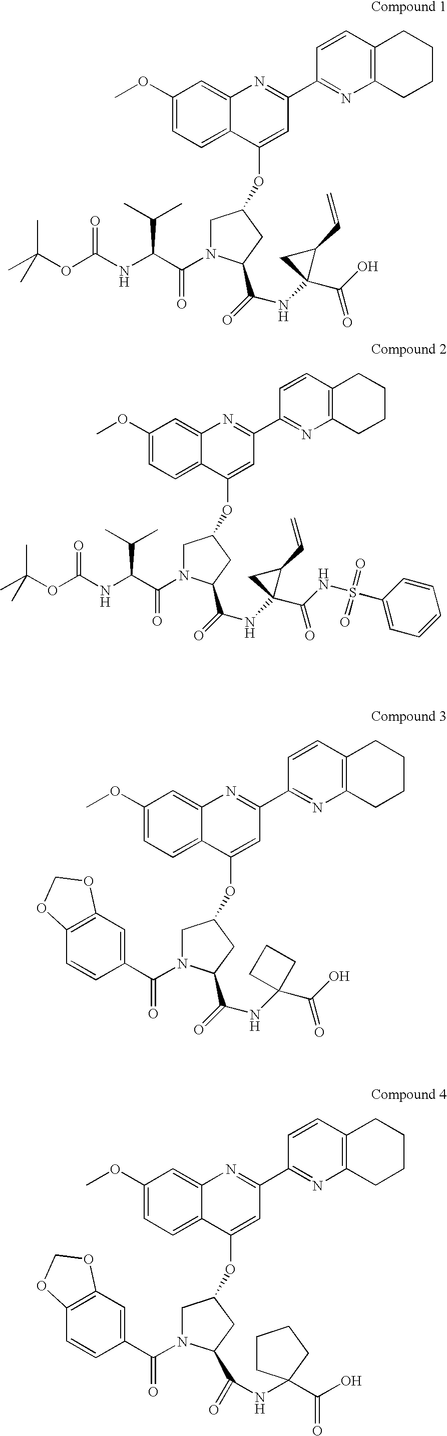

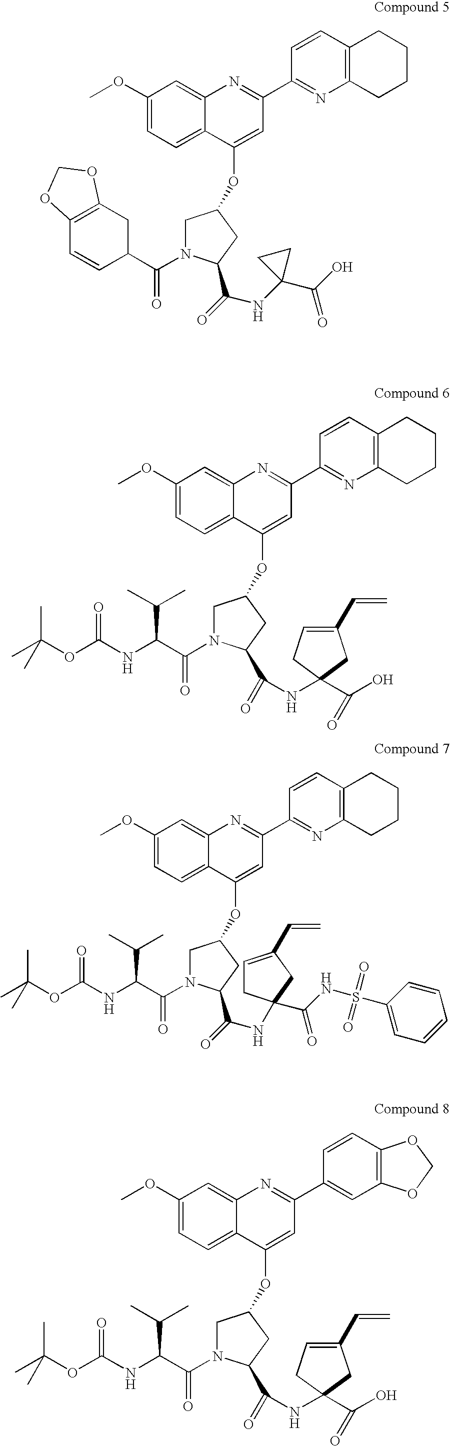

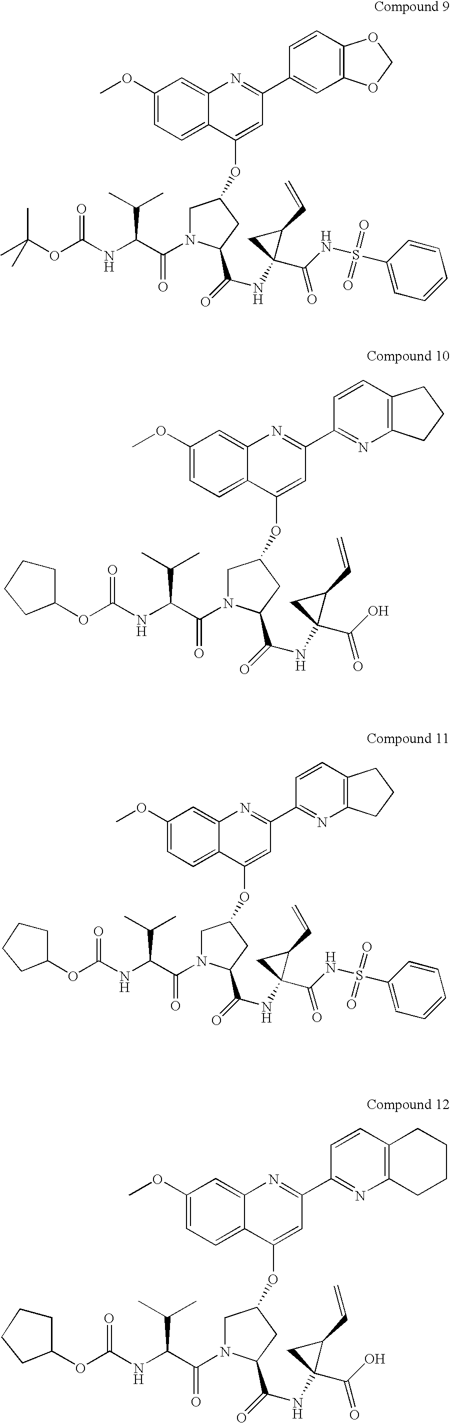

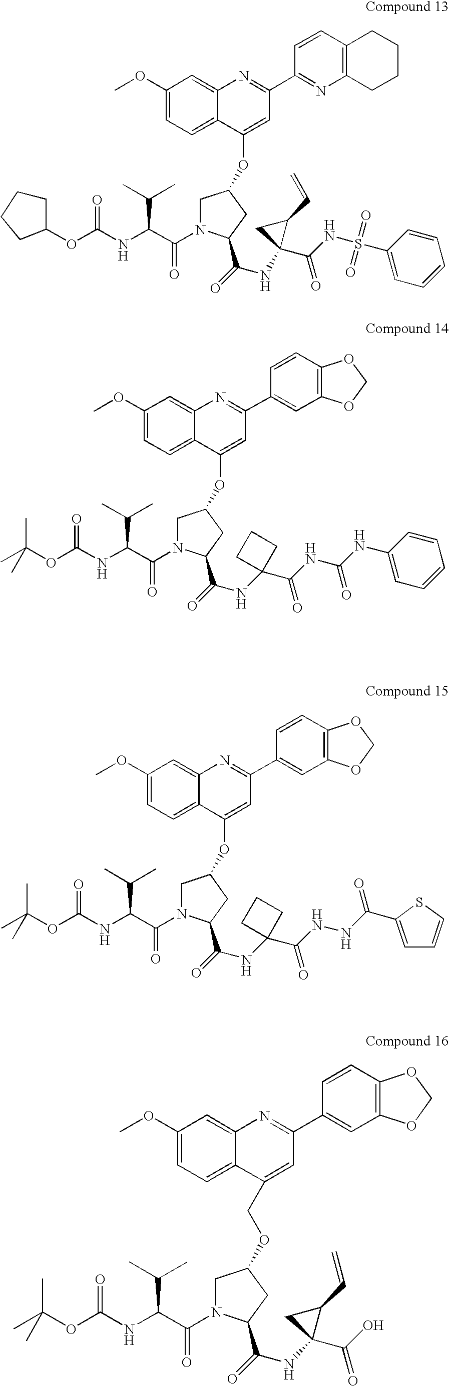

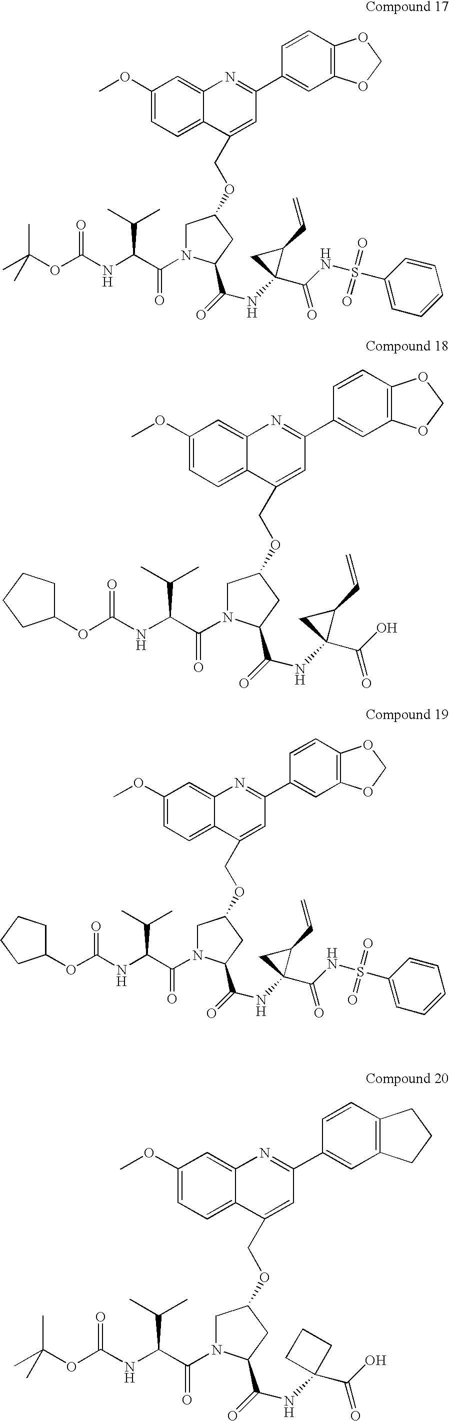

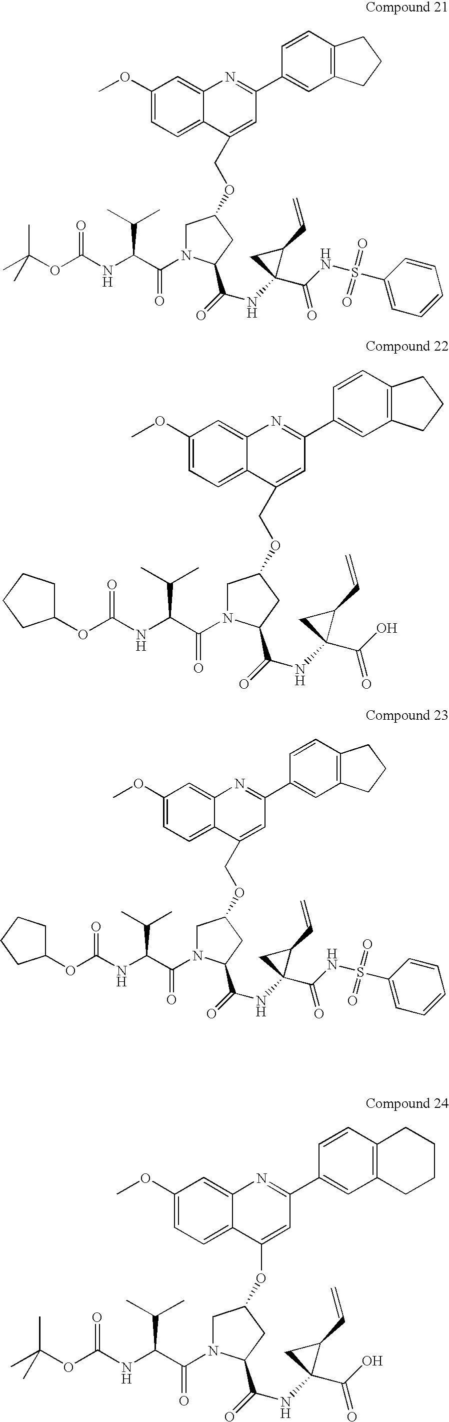

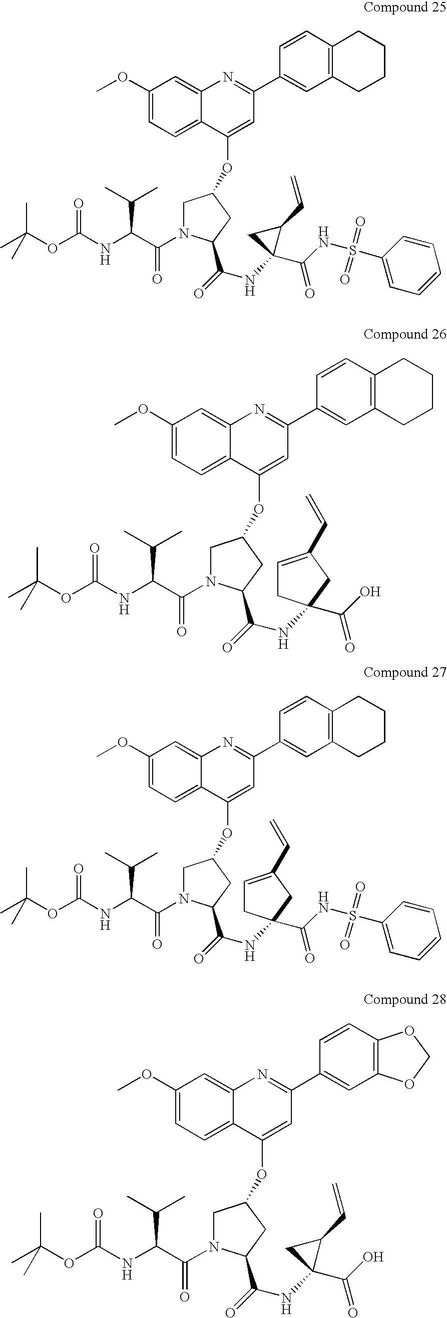

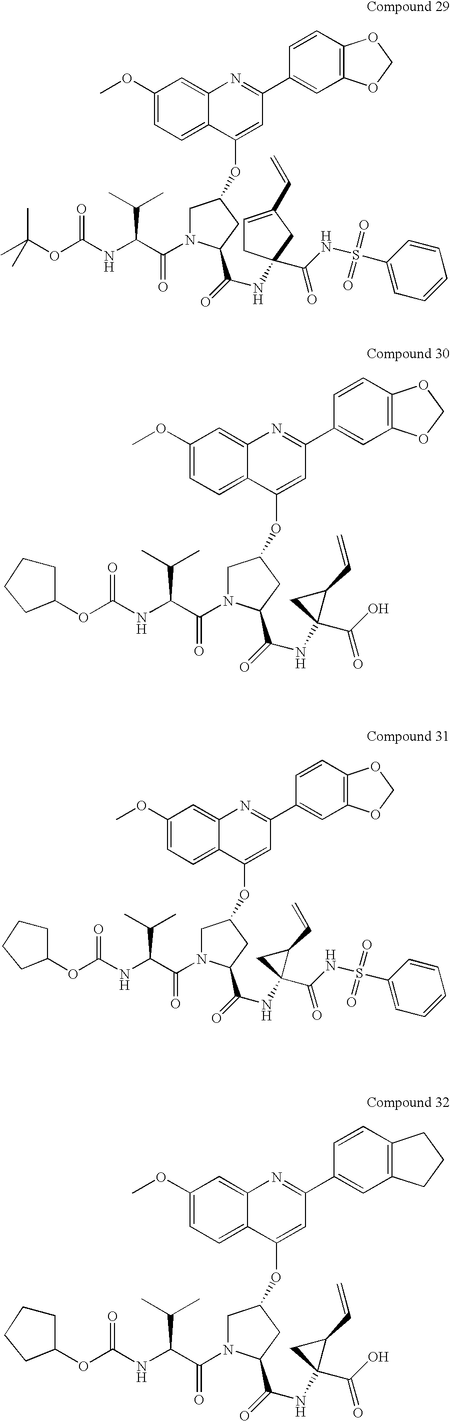

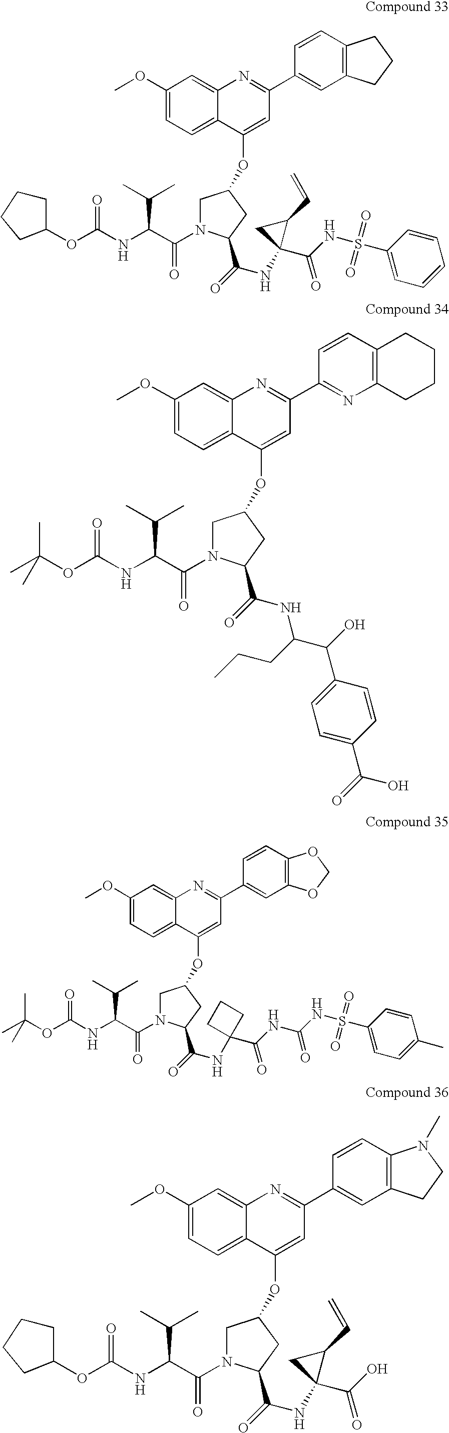

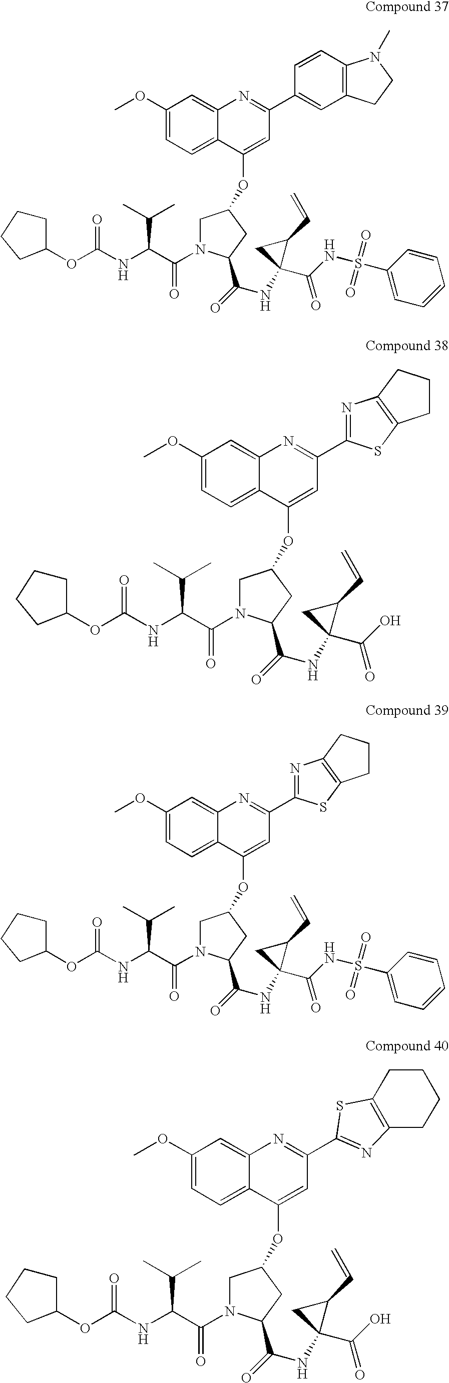

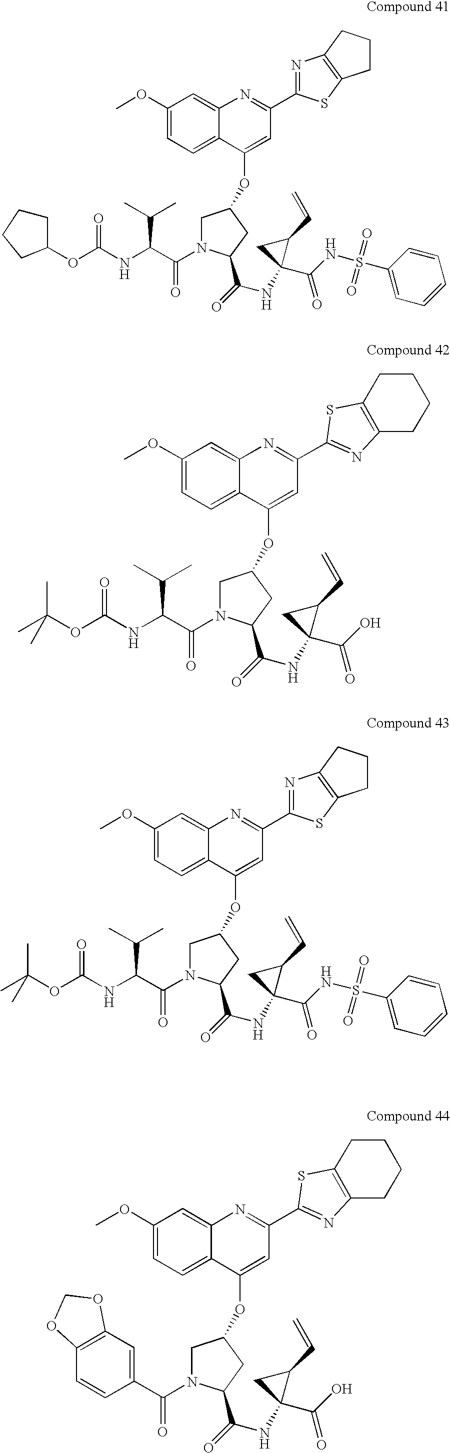

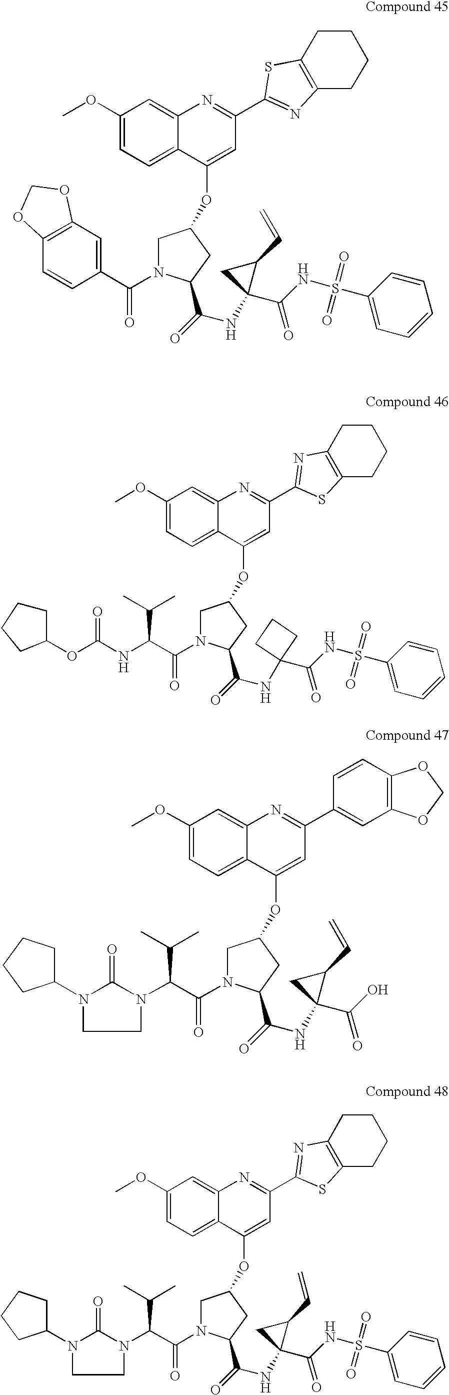

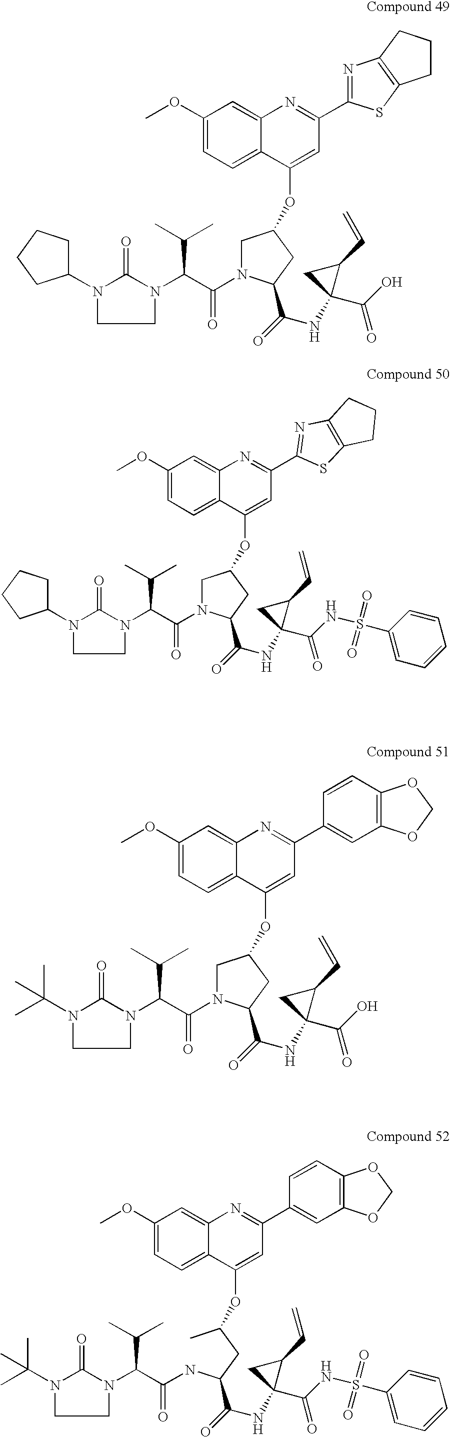

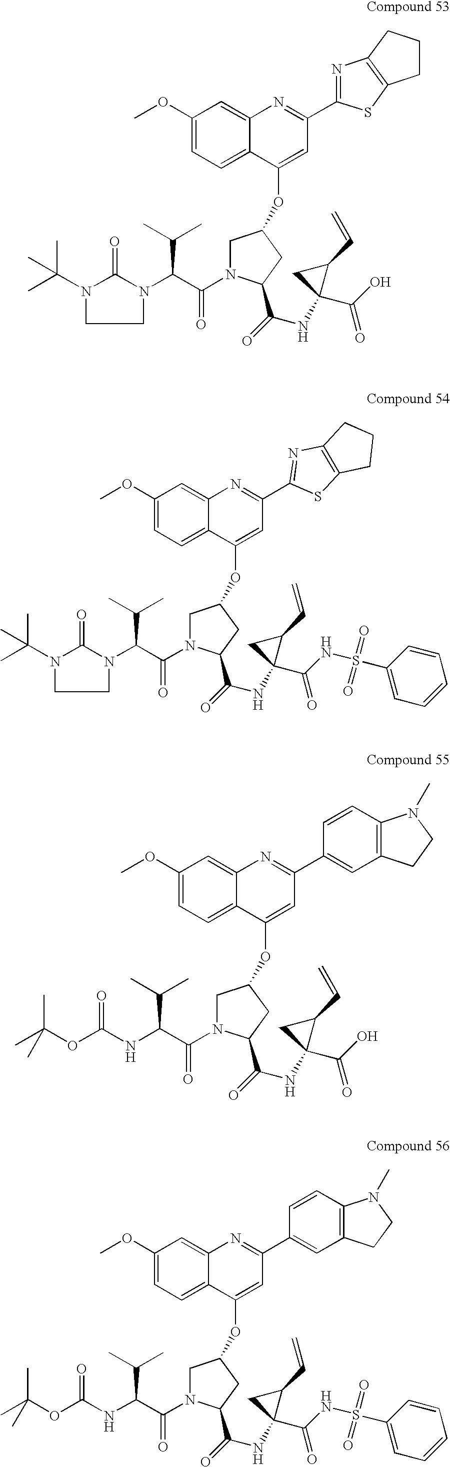

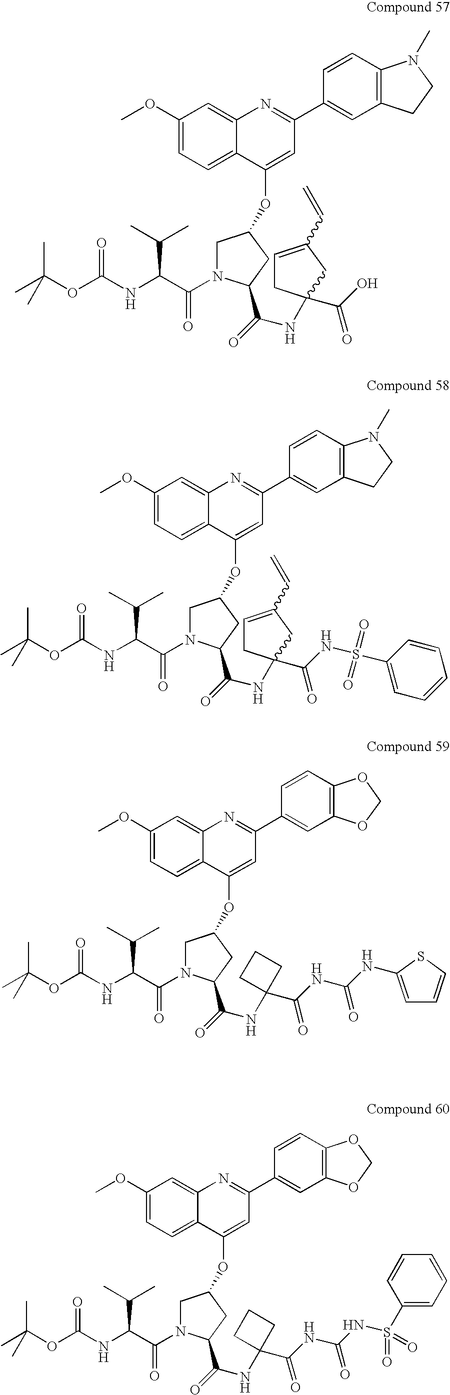

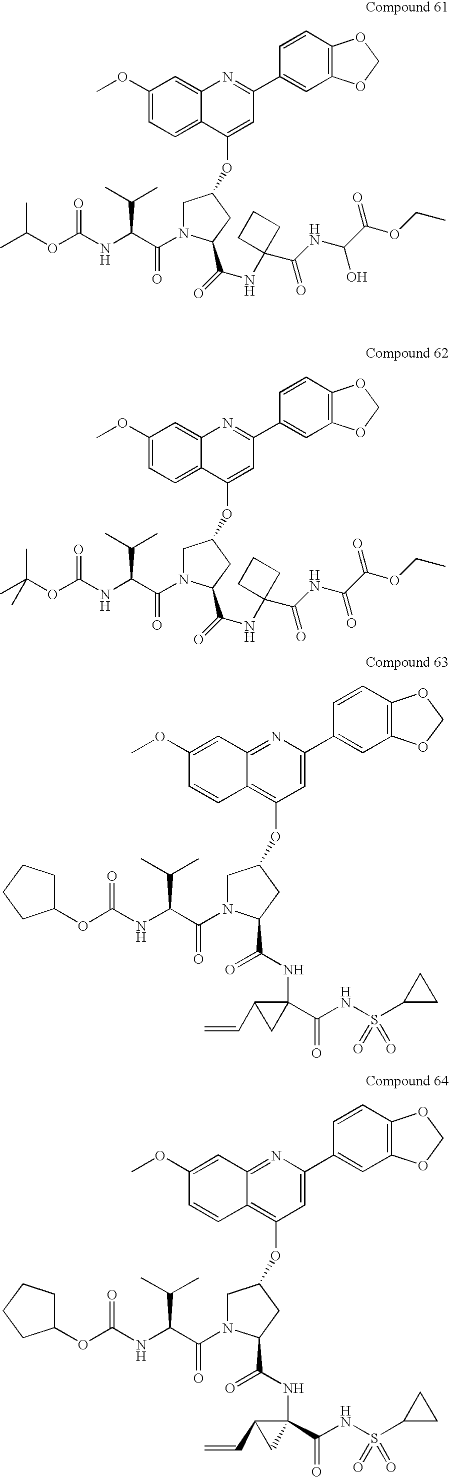

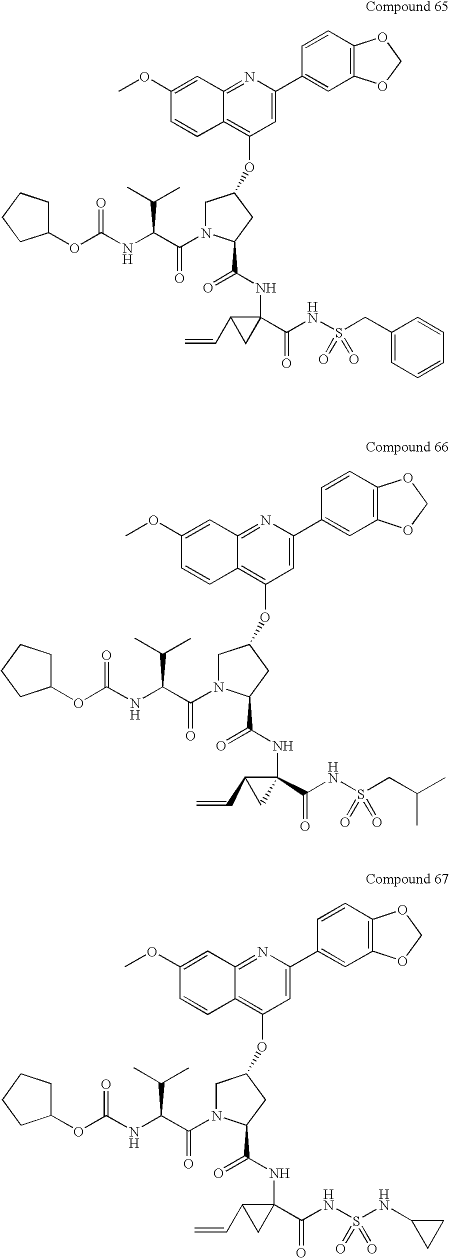

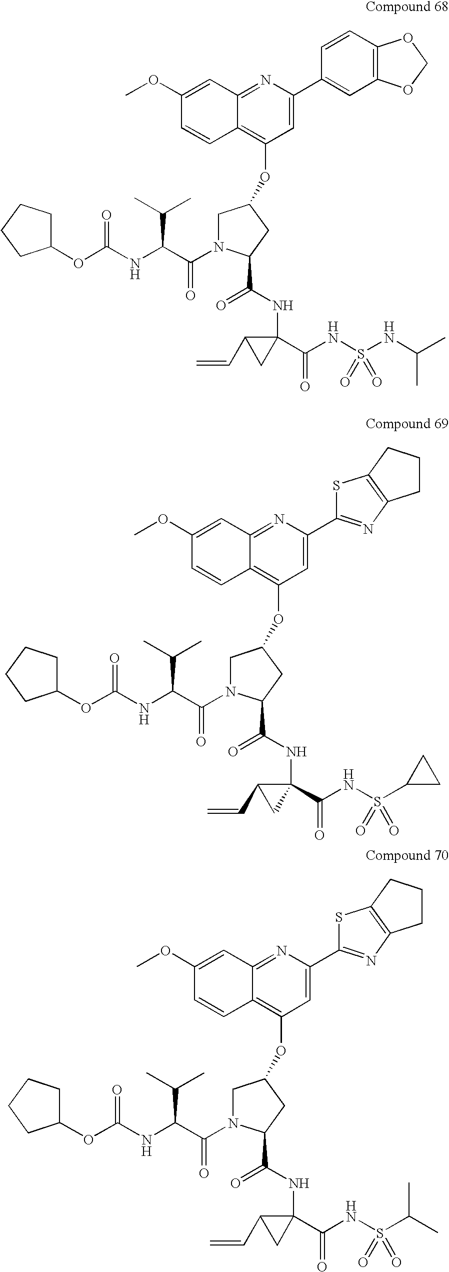

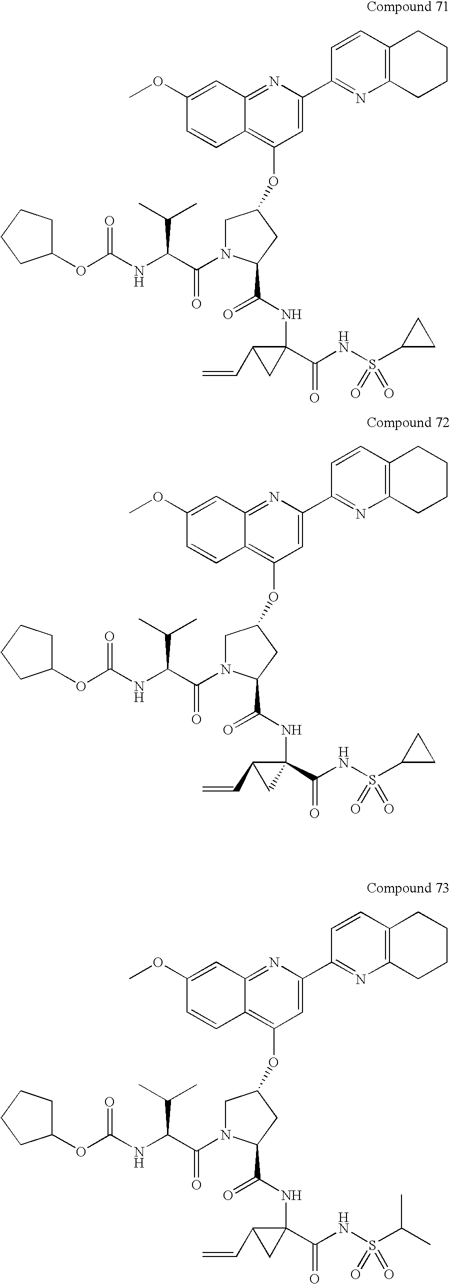

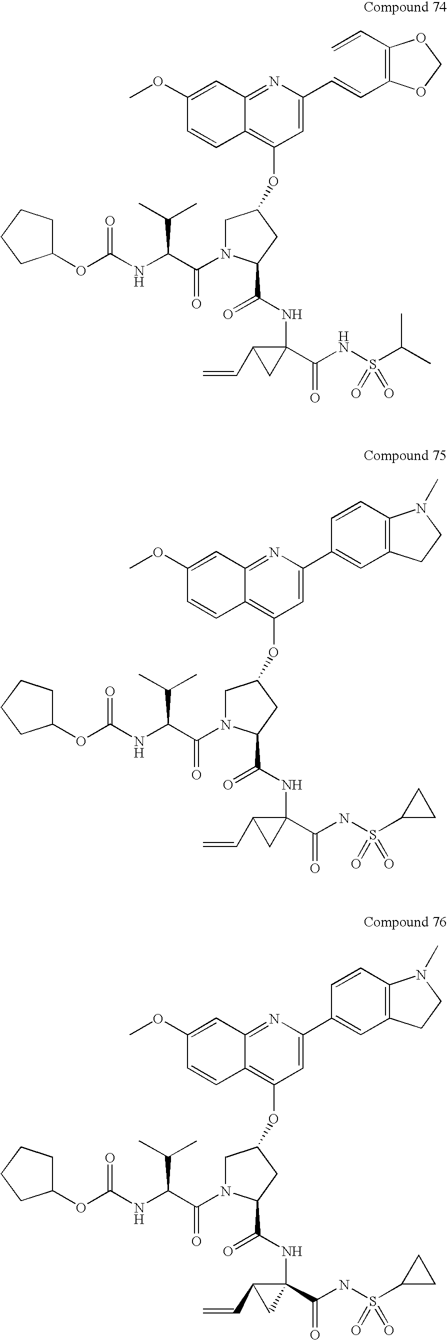

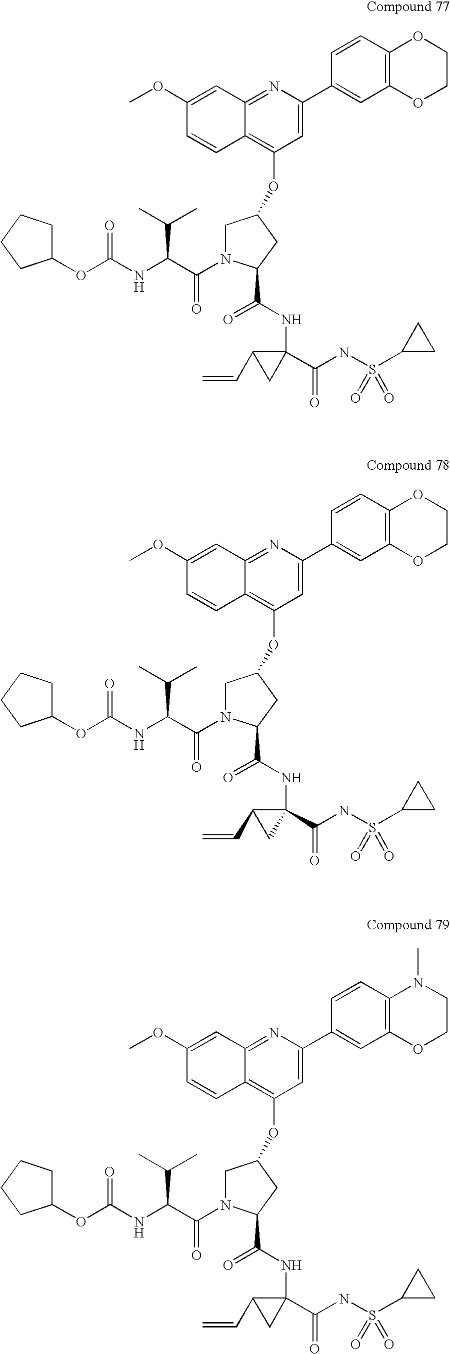

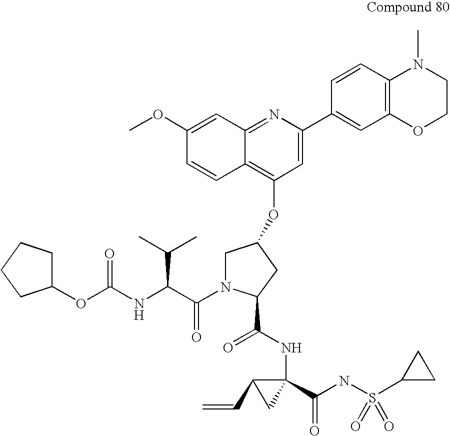

The compounds of formula (I) described above can be prepared by methods well known in the art. Examples 1-80 below provide detailed descriptions of how compounds 1-80 were actually prepared.

Scheme 1 shown below illustrates a typical route for synthesizing certain compounds of this invention. Variables A, B, Y, Z, n, R₁, R₃, and R₁₀ are defined in the Summary section above. Specifically, a substituted aniline compound can first undergo an acetylation reaction to form a 2-acetyl aniline. The compound thus obtained can react with a carboxylic acid containing an aryl or heteroaryl group to give an amide, which can undergo a ring closure reaction in the presence of a base (e.g., t-BuOK) to form a quinoline having a hydroxyl group. The quinoline can be treated with a chlorination agent (e.g., POCl₃) to convert the hydroxyl group to a chloride group. The chlorinated compound can subsequently react with t-Boc protected pyrrolidine containing a hydroxyl group and a carboxylic acid group to form an ether. The ether can then undergo a deprotection group to remove the t-Boc group and an esterification reaction to convert the carboxylic acid group to a carboxylate group. The compound thus obtained can react with an acid in the presence of peptide coupling agents to form an amide. The amide can undergo another hydrolysis reaction in the presence of a base (e.g., LiOH) to convert the carboxylate group back to the carboxylic acid group and then react with an amine containing a cycloalkyl ring and a methyl carboxylate group to form a diamide. The diamide can then be hydrolyzed to remove the methyl group to form certain compounds of the invention (e.g., compounds 1, 3-6, 8, 10, 12, 14, 24, 26, 28, 30, 32, 34, 36, 38, 40, 44, 47, 49, 51, 53, 55, and 57). The compounds thus obtained can be further modified (e.g., by reacting with an amine) to form certain other compounds of the invention (e.g., compounds 2, 7, 9, 11, 13, 15, 25, 27, 29, 31, 33, 35, 37, 39, 41-43, 45, 46, 48, 50, 52, 54, 6, and 58-80).

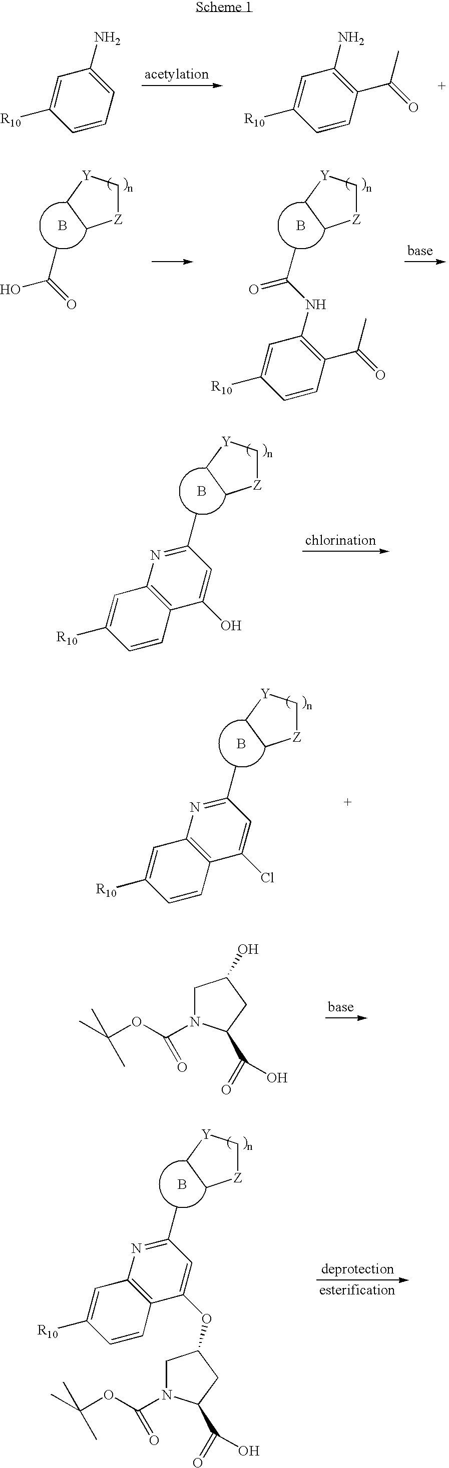

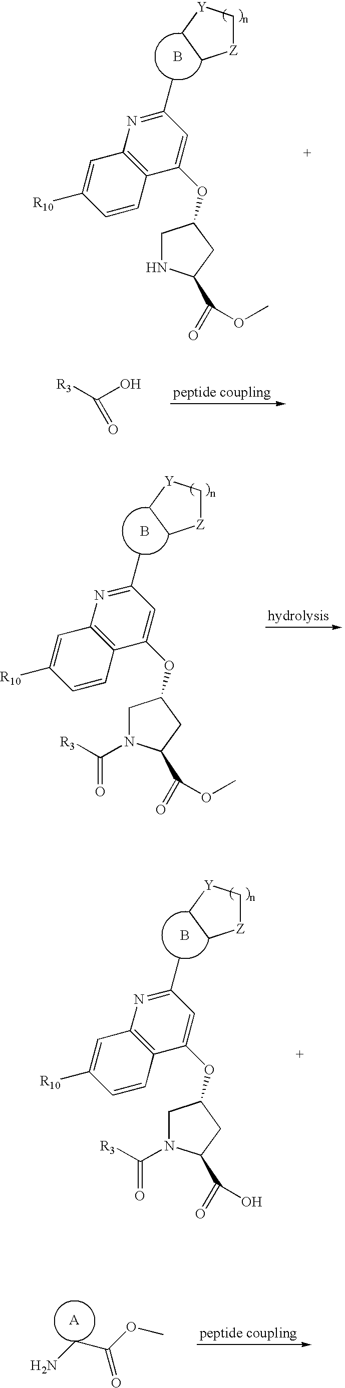

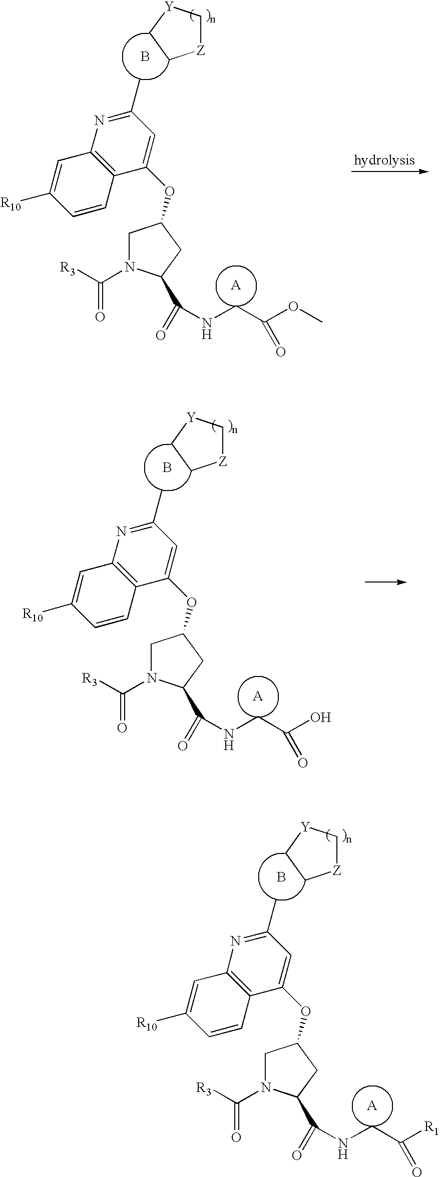

Some other compounds of the invention can be formed by synthetic routes similar to that described Scheme 1. For example, the ether described above can be formed by directly reacting the quinoline having a hydroxyl group with the t-Boc protected pyrrolidine having a hydroxyl group. As another example, to form compounds in which X recited in formula (I) is CH₂O or OCH₂ (e.g., compounds 16-23), a quinoline having a carboxylic acid group can be formed by reacting an isatin with 1-indan-ethanone in the presence of a base (e.g., KOH). The carboxylic acid group on the quinoline can then be reduced to a hydroxyl group, which can be converted to a chloride group. See, e.g., Example 20. The compound thus formed can then transformed into certain compounds of the invention following the same route illustrated in Scheme 1.

A compound synthesized above can be purified by a suitable method such as column chromatography, high-pressure liquid chromatography, or recrystallization.

Other compounds of formula (I) can be prepared using other suitable starting materials through the above synthetic routes and others known in the art. The methods described above may also additionally include steps, either before or after the steps described specifically herein, to add or remove suitable protecting groups in order to ultimately allow synthesis of the compounds of formula (I). In addition, various synthetic steps may be performed in an alternate sequence or order to give the desired compounds. Synthetic chemistry transformations and protecting group methodologies (protection and deprotection) useful in synthesizing applicable compounds of formula (I) are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The compounds mentioned herein may contain a non-aromatic double bond and one or more asymmetric centers. Thus, they can occur as racemates and racemic mixtures, single enantiomers, individual diastereomers, diastereomeric mixtures, tautomers, and cis- or trans-isomeric forms. All such isomeric forms are contemplated.

Also within the scope of this invention is a pharmaceutical composition containing an effective amount of at least one compound of formula (I) described above and a pharmaceutical acceptable carrier. Further, this invention covers a method of administering an effective amount of one or more of the compounds of formula (I) to a patient having a HCV infection. “An effective amount” refers to the amount of an active compound of formula (I) that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the method of the present invention, a composition having one or more compounds of formula (I) can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active compounds of formula (I) can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound of formula (I). Examples of other carriers include colloidal silicon oxide, magnesium stearate, cellulose, sodium lauryl sulfate, and D&C Yellow # 10.

The compounds of formula (I) described above can be preliminarily screened for their efficacy in treating HCV infection by an in vitro assay (Example 63 below) and then confirmed by animal experiments and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1

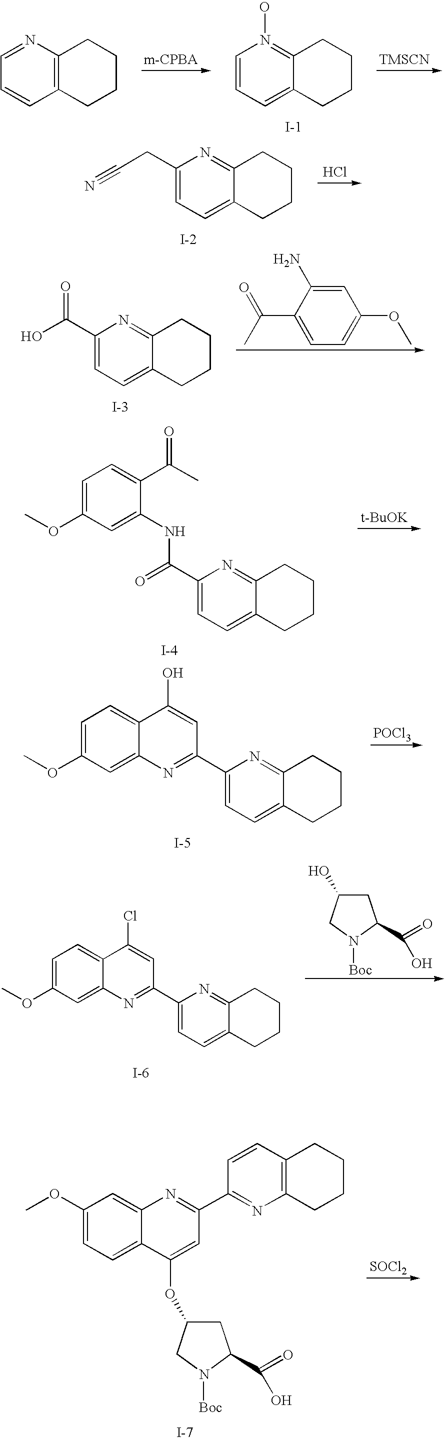

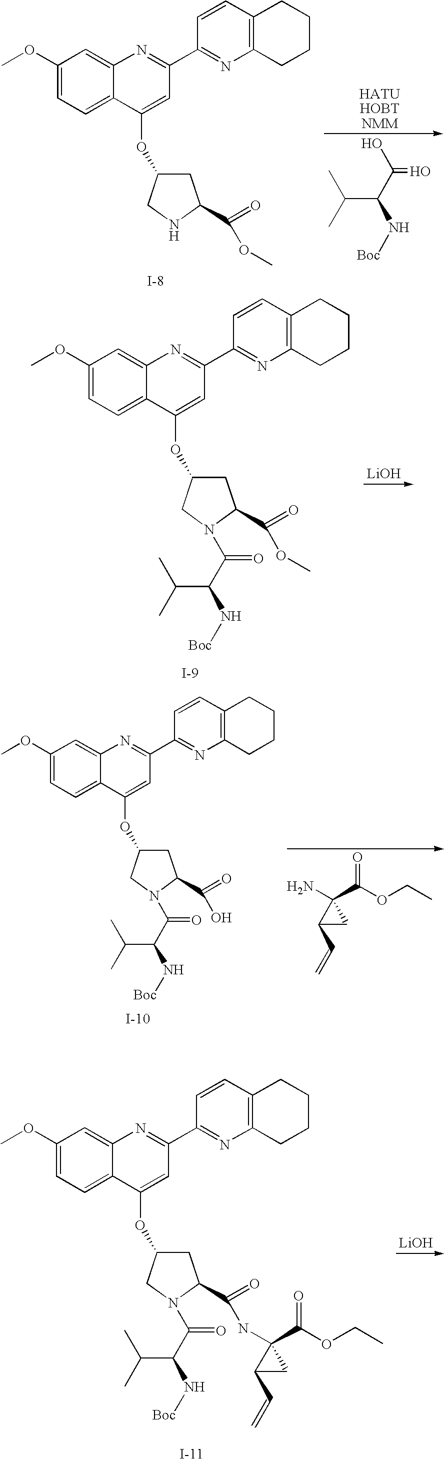

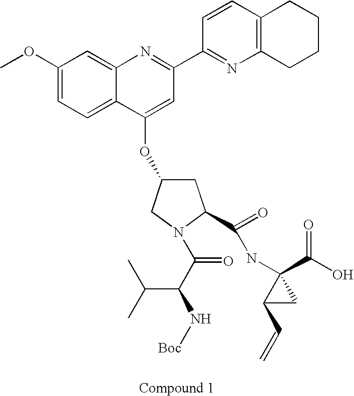

3-Chloroperoxybenzoic acid (m-CPBA, 10.5 g, 45 mmol) was added to a solution of 5,6,7,8-tetrahydroquinoline (5.0 g, 37.5 mmol) in CH₂Cl₂ (350 mL) at room temperature. After the reaction mixture was stirred overnight, it was quenched by a 1M aqueous sodium hydroxide solution and then a saturated aqueous NaCl solution. CH₂Cl₂ extracted the mixture. The combined organic layers were dried and concentrated. The residue thus obtained was purified by silica gel column chromatography to afford compound I-1 (5.5 g, 98%). ESI-MS (M+H⁺)=150.

To a solution of compound I-1 (10 g, 67 mmol) in CH₂Cl₂ (134 mL) were added trimethylsilyl cyanide (TMSCN, 18.5 mL, 147.4 mmol) and dimethylcarbamyl chloride (13.7 mL, 147.4 mmol) at room temperature. After the reaction mixture was stirred overnight, it was quenched by a 3M aqueous sodium hydroxide (350 mL). The mixture was vigorously stirred for 10 minutes and extracted with CH₂Cl₂ (200 mL×6). The combined organic layers were dried and concentrated. The residue thus obtained was purified by silica gel column chromatography to afford compound I-2 (6.5 g, 61%). ESI-MS (M+H⁺)=159.

A solution of compound I-2 (6.5 g, 41 mmol) in a 6M aqueous hydrochloric acid solution (150 mL, 24 eq.) was refluxed for 4 days. The solution was concentrated to give crude compound I-3 (12 g), which was used in the next step without further purification. ESI-MS (M+H⁺)=178.

A mixture of crude compound I-3 (3.1 g) and 1-(2-amino-4-methoxyphenyl)ethanone (1.5 g, 9 mmol) in pyridine (75 mL, 0.12 M) was cooled to −30° C. Phosphorus oxychloride (2.8 mL, 30 mmol, 3 eq.) was added dropwise over a period of 5 minutes. After the reaction mixture was stirred at −30° C. for 1 hour, the cooling bath was removed and the mixture was allowed to warm-up to room temperature. After the mixture was stirred for another 2 hours, it was poured into ice water. The pH was adjusted to 11 with an 2N aqueous NaOH. After the mixture was extracted with CH₂Cl₂, the organic layer was dried over anhydrous MgSO₄, filtered, and concentrated under vacuum. The crude product thus obtained was purified by silica gel column chromatography (30% EtOAc in hexane) to give compound I-4 (1.9 g, 65%). ESI-MS (M+H⁺)=325.

t-BuOK (2.6 g, 23.5 mmol, 4 eq.) was added to a suspension of compound I-4 (1.9 g, 5.9 mmol) in anhydrous tBuOH (45 mL). After the reaction mixture was heated under reflux for 2 hours, it was then cooled to room temperature and acidified by adding HCl (4N in dioxane, 1.5 eq.). The mixture was then concentrated under vacuum to give crude compound I-5, which was used in the next step without further purification. ESI-MS (M+H⁺)=307.

A solution of I-5 (5.9 mmol) in POCl₃ (5.4 mL, 59 mmol) was heated to reflux for 1.5 hours. After removal of POCl₃ in vacuum, the residue was quenched by 2N NaOH to pH>7, stirred for 15 minutes, and then extracted by CH₂Cl₂. The organic layer was dried over anhydrous MgSO₄, filtered, and concentrated under vacuum. The crude product thus obtained was then purified by silica gel column chromatography to give compound I-6 (1.2 g, 64% from 3 steps). ¹H NMR (CDCl₃) δ 8.51 (s, 1H), 8.28 (d, J=7.8 Hz, 1H), 8.11 (d, J=9.2 Hz, 1H), 7.52 (d, J=8.1 Hz, 1H), 7.48 (s, 1H), 7.27-7.23 (m, 2H), 3.99 (s, 3H), 3.03 (t, J=6.3 Hz, 2H), 2.85 (t, J=6.0 Hz, 2H), 1.98-1.62 (m, 4H). ESI-MS (M+H⁺)=325.

To a suspension of Boc-4R-hydroxyproline (1.9 g, 8.2 mmol) in DMSO (25 mL) was added t-BuOK (2.3 g, 14.7 mmol) at 0° C. The mixture was then allowed to return to room temperature. After the reaction mixture was stirred for 1 hour, and compound I-6 (2.67 g, 8.2 mmol) was added in three portions over 1 hour. The reaction mixture was stirred for 1 day and then was poured into cold water. The aqueous solution was acidified to pH 4.6 and filtered to give crude compound I-7, which was used in the next step without further purification. ESI-MS (M+H⁺)=520.

To a solution of crude compound I-7 (2.7 g, 5.2 mmol) in MeOH (100 mL) was added SOCl₂ (3.1 g, 26 mmol) at room temperature. The reaction mixture was heated to reflux for 1 hour. MeOH and SOCl₂ were removed to give crude compound I-8, which was used in the next step without further purification. ESI-MS (M+H⁺)=434.

N-Methylmorpholine (NMM, 3.5 g, 34.6 mmol) was added to a solution of crude compound I-8 (3.0 g, 6.9 mmol), 2-(1H-7-Azabenzotriazol-1-yl)-1,1,3,3-tetramethyl uronium hexafluoro-phosphate methanaminium (HATU, 3.95 g, 10.4 mmol), N-Hydroxybenzotriazole (HOBT, 1.4 g, 10.4 mmol) and Boc-VAL-OH (1.5 g, 6.9 mmol) in CH₂Cl₂ (50 mL) at room temperature. After the mixture was stirred overnight, it was concentrated under vacuum. The residue thus obtained was purified by silica gel column chromatography to give compound I-9 (3.2 g, 73%). ESI-MS (M+H⁺)=633.

To a solution of I-9 (3.2 g, 5.1 mmol) in THF (50 mL) was added 0.5 M LiOH (20 mL, 10.1 mmol) at room temperature. After the reaction mixture was stirred overnight, it was acidified by 10% HCl to pH<7 and concentrated under vacuum to give a yellow solid. The solid was washed by water and filtered to give compound I-10. ESI-MS (M+H⁺)=619.

NMM (0.12 g, 1.21 mmol) was added to a solution of compound I-10 (0.25 g, 0.41 mmol), HATU (0.31 g, 0.81 mmol), HOBT (0.082 g, 0.61 mmol) and ethyl 1-amino-2-vinylcyclopropanecarboxylate (0.076 g, 0.41 mmol) in CH₂Cl₂ (10 mL) at room temperature. After the reaction mixture was stirred overnight, it was concentrated under vacuum. The residue was purified by silica gel column chromatography to give compound I-11 (0.22 g, 72%). ESI-MS (M+H⁺)=756.

To a solution of I-11 (0.22 g, 0.29 mmol) in THF (10 mL) were added 0.5 M LiOH (1.2 mL, 0.58 mmol) and MeOH (1 mL) at room temperature. After the reaction mixture was stirred overnight, it was acidified by 10% HCl to pH<7, and concentrated under vacuum to give a yellow solid. The solid was washed by water and filtered to give Compound 1 (0.17 g, 80%). ¹H NMR (CD₃OD) δ 8.37-8.32 (m, 2H), 7.94 (s, 1H), 7.88-7.85 (m, 2H), 7.37 (dd, J=2.4 Hz, 9 Hz, 1H), 5.87-5.78 (m, 2H), 5.27 (d, J=16.5 Hz, 1H), 5.10 (dd, J=1.8 Hz, 10.2 Hz, 1H), 4.76-4.66 (m, 2H), 3.96 (s, 3H), 4.19-3.96 (m, 2H), 3.16 (t, J=6.2 Hz, 2H), 2.98 (t, J=6.2 Hz, 2H), 2.84-2.78 (m, 1H), 2.64-2.55 (m, 1H), 2.24-2.21 (m, 1H), 2.03-1.92 (m, 6H), 1.73-1.68 (m, 1H), 1.15 (s, 9H), 1.02-0.96 (m, 6H). ESI-MS (M+H⁺)=728.

### EXAMPLE 2

### Preparation of Compound 2

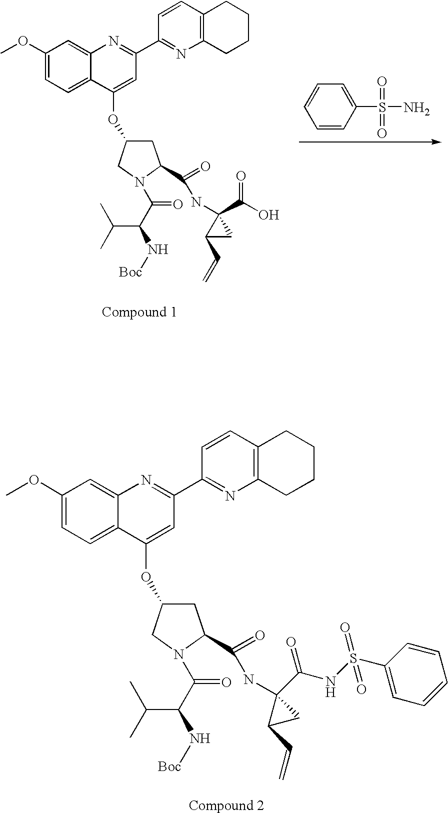

A solution of Compound 1 (0.17 g, 0.23 mmol), HATU (0.18 g, 0.47 mmol) and 4-dimethylaminopyridine (DMAP, 0.14 g, 1.17 mmol) in CH₂Cl₂ (5 mL) was stirred at room temperature for 1 hour, followed by dropwise addition of benzenesulfonamide (0.073 g, 0.47 mmol), diisopropylethylamine (DIPEA, 0.18 mL, 1.4 mmol) and 1,8-diazabicyclo[5,4,0]undec-7-ene (DBU, 0.18 g, 1.17 mmol) over a period of 15 minutes. After the reaction mixture was stirred at room temperature overnight, the solvent was removed by vacuum. The residue thus obtained was purified by silica gel column chromatography to give Compound 2. (30 mg, 15%). ¹H NMR (CDCl₃) δ 8.30 (d, J=7.8 Hz, 1H), 8.02-8.76 (m, 2H), 7.58 (s, 1H), 7.58-7.42 (m, 5H), 7.09 (s, 1H), 7.03 (d, J=7.8 Hz, 1H), 5.53-5.47 (m, 2H), 5.12 (d, J=16.8 Hz, 1H), 4.95 (d, J=10.2 Hz, 1H), 4.56 (d, J=11.2 Hz, 1H), 4.45 (m, 1H), 4.20 (m, 1H), 4.11 (d, J=7.8 Hz, 1H), 3.96 (s, 3H), 3.01 (t, J=6.2 Hz, 2H), 2.85 (t, J=6.2 Hz, 2H), 2.65-2.63 (m, 1H), 2.55-2.50 (m, 1H), 2.19-2.16 (m, 1H), 2.03-1.80 (m, 7H), 1.25 (s, 9H), 1.00 (d, J=6.8 Hz, 3H), 0.98 (d, J=6.8 Hz, 3H). ESI-MS (M+H⁺)=867.

### EXAMPLE 3

### Preparation of Compound 3

Compound 3 was prepared (0.12 g, 80%) in a manner similar to that described in Example 1. ¹H NMR (DMSO) δ 8.24 (m, 2H), 7.86 (d, J=7.8 Hz, 1H), 7.75 (s, 1H), 7.60 (d, J=7.8 Hz, 1H), 7.35 (s, 1H), 7.20-6.89 (m, 3H), 6.02 (s, 2H), 5.50-5.40 (brs, 1H), 4.71 (m, 1H), 4.05 (m, 1H), 3.90 (s, 3H), 3.70 (m, 1H), 2.91 (brs, 2H), 2.79 (brs, 2H), 2.70-2.60 (m, 3H), 2.40-2.30 (m, 1H), 2.23-2.07 (brs, 2H), 2.00-1.75 (m, 6H). ESI-MS (M+H⁺)=665.

### EXAMPLE 4

### Preparation of Compound 4

Compound 4 was prepared (0.13 g, 83%) in a manner similar to the described in Example 1. ¹H NMR (CD₃OD) δ 8.40-8.23 (m, 1H), 8.21-8.17 (m, 1H), 7.90-7.79 (m, 3H), 7.60-7.40 (m, 1H), 7.20-6.80 (m, 3H), 5.98 (s, 2H), 5.79 (brs, 1H), 4.95-4.75 (m, 1H), 4.33-4.25 (m, 1H), 4.03 (s, 3H), 4.01-3.98 (m, 1H), 3.13 (m, 2H), 2.97 (m, 2H), 2.90-2.80 (m, 1H), 2.70-2.56 (m, 1H), 2.65-2.62 (m, 1H), 2.40-1.60 (m, 12H). ESI-MS (M+H⁺)=678.

### EXAMPLE 5

### Preparation of Compound 5

Compound 5 was prepared (0.12 g, 80%) in a manner similar to that described in Example 1. ¹H NMR (CD₃OD) δ 8.26 (d, J=8.1 Hz, 1H), 8.11 (d, J=9.0 Hz, 1H), 7.88-7.74 (m, 3H), 7.38 (d, J=8.1 Hz, 1H), 7.17-6.85 (m, 3H), 5.98 (s, 2H), 5.76 (brs, 1H), 4.95-4.75 (m, 1H), 4.33-4.25 (m, 1H), 4.03 (s, 3H), 4.01-3.98 (m, 1H), 3.11 (m, 2H), 2.95 (m, 2H), 2.93-2.80 (m, 1H), 2.68 (m, 1H), 2.70-2.67 (m, 1H), 2.01-1.93 (m, 4H), 1.58-1.48 (m, 2H), 1.28-1.15 (m, 2H). ESI-MS (M+H⁺)=650.

### EXAMPLE 6

### Preparation of Compound 6

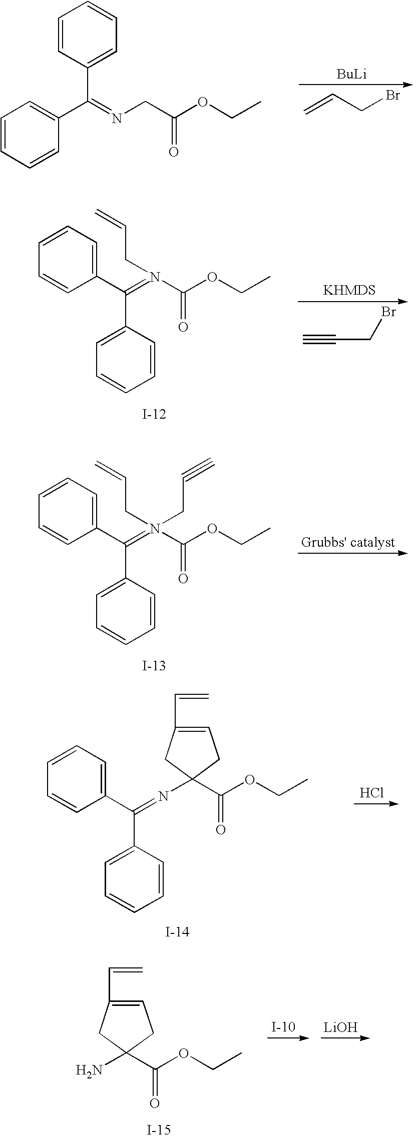

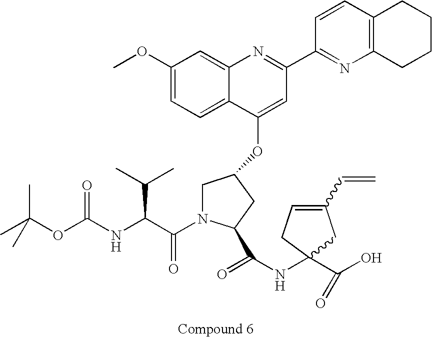

Under a nitrogen atmosphere at −78° C., 1,3-dimethyl-3,4,5,6-tetrahydro-pyrimidinone (DMPU, 4.79 g, 37.4 mmol) and N-(diphenylmethylene)glycine ethyl ester (10 g, 37.4 mmol) were added to a solution of 2.5 M BuLi (15 mL) in hexane and diisopropyl amine (3.79 g, 37.4 mmol) in THF. After the mixture was stirring for 30 minutes, 4-bromo-1-butene (4.45 g, 37.4 mmol) was added. The mixture was stirred continuously for another 2 hours at −78° C. and then allowed to warm up to room temperature overnight. The mixture was quenched by a saturated NH₄Cl aqueous solution. After the solution were concentrated, the aqueous layer was extracted by CH₂Cl₂. The organic layers were combined, dried, and concentrated under vacuum. The residue thus obtained was then purified by silica gel column chromatography to afford compound I-12 (11.3 g, 92%). ESI-MS (M+H⁺)=308.

A mixture of potassium hexamethyldisilazane (KHMDS, 29.1 mL, 14.5 mmol) and DMPU (1.87 g, 14.5 mmol) in THF was cooled to −78° C., follow by addition of compound I-12 (3 g, 9.7 mmol). The mixture was then allowed to warm up to room temperature for 30 minutes. After propargyl bromide was subsequently added at −78° C., the solution was again allowed to warm to room temperature and stirred for overnight. The reaction mixture was quenched by H₂O and extracted by CH₂Cl₂. The combined organic extracts were washed with brine, dried with anhydrous MgSO₄, filtered, and concentrated under vacuum. The crude product thus obtained was purified by silica gel column chromatography to give compound I-13 (2.9 g, 86%). ESI-MS (M+H⁺)=346.

The first generation of the Grubbs' catalyst (benzylidene-bis(tricyclohexylphosphine) dichlororuthenium, Aldrich, St. Louis, Mo.; 0.15 g, 0.17 mmol) was added to a solution of compound I-13 (0.6 g, 1.7 mmol) in CH₂Cl₂ (30 mL) while stirring at room temperature. After the reaction mixture was stirred for 3 days, it was filtrated and concentrated under vacuum. The crude product thus obtained was purified by silica gel column chromatography to give I-14 (0.26 g, 44%). ¹H NMR (CDCl₃) δ 7.82-7.14 (m, 10H), 6.49 (dd, J=17.8 Hz, 9.8 Hz, 1H), 5.62 (d, J=0.6 Hz, 1H), 5.08 (s, 1H), 5.04 (d, J=4.8 Hz, 1H), 3.69 (q, J=7.1 Hz, 2H), 3.23-3.15 (m, 2H), 3.06-2.95 (m, 2H), 1.11 (t, J=7.2 Hz, 4H). ESI-MS (M+H⁺)=346.

To a solution of compound I-14 in THF/CH₂Cl₂ (5 mL/5 mL) was added 10% HCl at room temperature. After the reaction mixture was stirred for 1.5 hours, it was quenched by a saturated NaHCO₃ aqueous solution to adjust pH>7 and then concentrated under vacuum. The residue was then washed by CH₂Cl₂. The organic layer was collected and concentrated under vacuum to give crude I-15. ESI-MS (M+H⁺)=182.

Compound 6 was prepared (0.15 g, 83%) in a manner similar to that described in Example 1 except that ethyl 1-amino-2-vinylcyclopropanecarboxylate used in the preparation of compound I-11 was replaced with compound I-15. ¹H NMR (CDCl₃) δ 8.38-8.33 (m, 2H), 7.95 (s, 1H), 7.88-7.86 (m, 2H), 7.39-7.35 (dd, J=9.6 Hz, 2.1 Hz, 1H), 6.62-6.50 (m, 1H), 5.86 (s, 1H), 5.75 (d, J=16.8 Hz, 1H), 5.13-5.06 (m, 2H), 4.96-4.80 (m, 2H), 4.18-4.02 (m, 4H), 3.94 (d, J=7.8 Hz, 1H), 3.42-3.20 (m, 2H), 3.16 (t, J=6.2 Hz, 2H), 3.04-2.99 (m, 3H), 2.85-2.81 (m, 3H), 2.62-2.50 (m, 1H), 2.03-1.94 (m, 4H), 1.13 (s, 9H), 0.99 (d, J=6.6 Hz, 3H), 0.95 (d, J=6.3 Hz, 3H). ESI-MS (M+H⁺)=754.

### EXAMPLE 7

### Preparation of Compound 7

Compound 7 was prepared (16 g, 13%) in a manner similar to that described in Example 2. ¹H NMR (CDCl₃) δ 8.32 (d, J=6.8 Hz, 1H), 8.06-8.01 (m, 2H), 7.87-7.81 (m, 2H), 7.60-7.49 (m, 5H), 7.07 (d, J=9.6 Hz, 1H), 6.38 (dd, J=17.2 Hz, 5.1 Hz, 1H), 5.60 (m, 1H), 5.49-5.41 (m, 1H), 5.29 (m, 1H), 5.05-4.95 (m, 1H), 4.57-4.47 (m, 2H), 4.18-4.15 (m, 2H), 3.96 (s, 3H), 3.03 (m, 2H), 2.84 (m, 2H), 2.72-2.61 (m, 2H), 2.40-2.00 (m, 5H), 1.94-1.86 (m, 4H), 1.25 (s, 9H), 1.10-1.00 (m, 6H). ESI-MS (M+H⁺)=893.

### EXAMPLE 8

### Preparation of Compound 8

Compound 8 was prepared (0.15 g, 83%) in a manner similar to that described in Example 1. ¹H NMR (CD₃OD+CDCl₃) δ 8.16-8.09 (m, 2H), 7.63 (s, 1H), 7.37-7.31 (m, 2H), 7.20-7.13 (m, 2H), 6.46-6.38 (m, 1H), 5.79 (s, 2H), 5.54 (s, 1H), 5.22 (d, J=7.8 Hz, 1H), 5.04-4.95 (m, 2H), 4.81 (m, 1H), 4.35 (d, J=9.6 Hz, 1H), 4.22 (m, 1H), 4.05 (s, 3H), 3.74 (m, 1H), 3.16-3.11 (m, 2H), 3.06-2.61 (m, 3H), 2.05-2.00 (m, 1H), 1.85 (m, 1H), 1.34 (s, 9H), 0.97 (d, J=3.2 Hz, 3H), 0.90 (d, J=6.3 Hz, 3H). ESI-MS (M+H⁺)=743.

### EXAMPLE 9

### Preparation of Compound 9

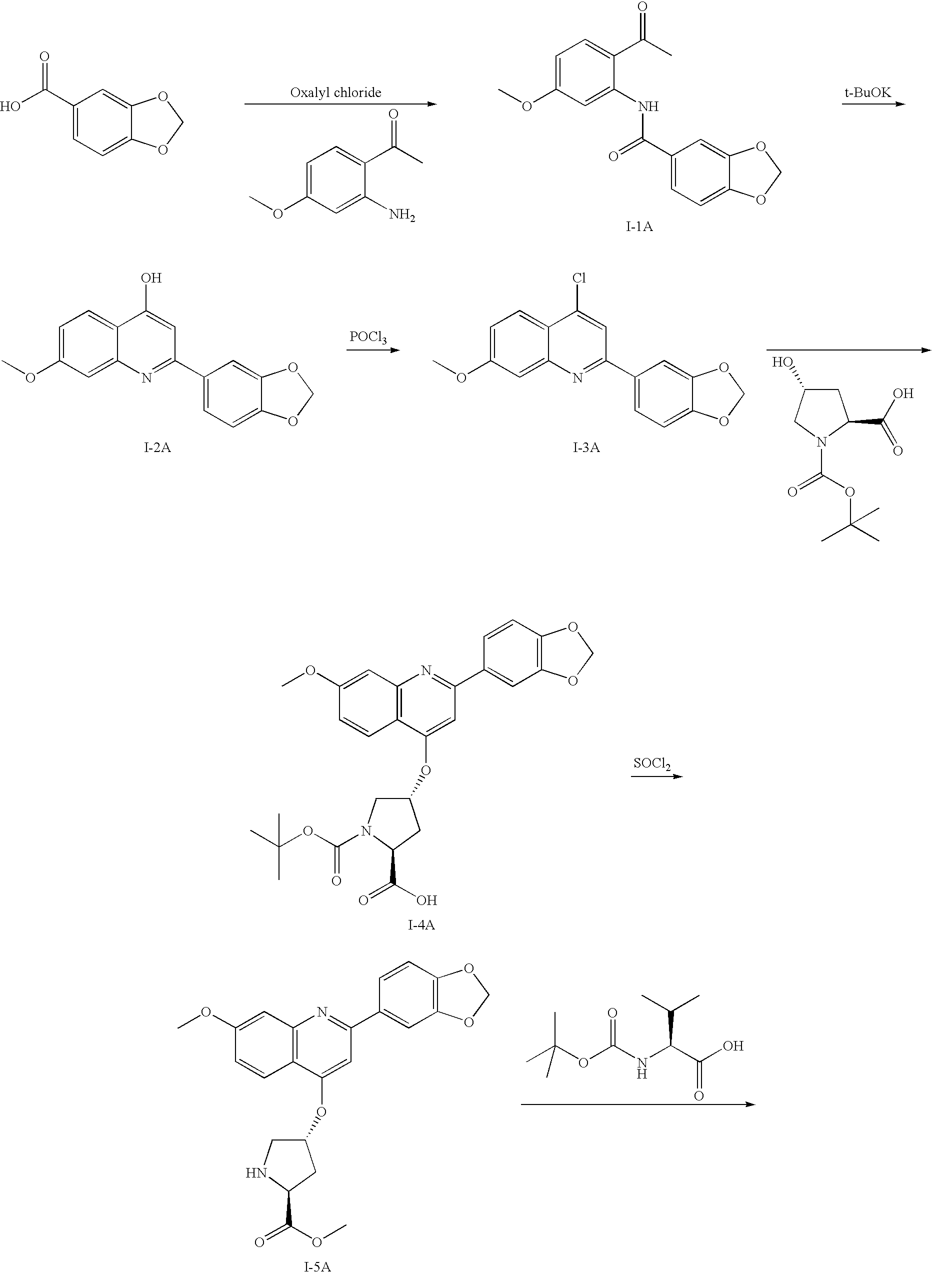

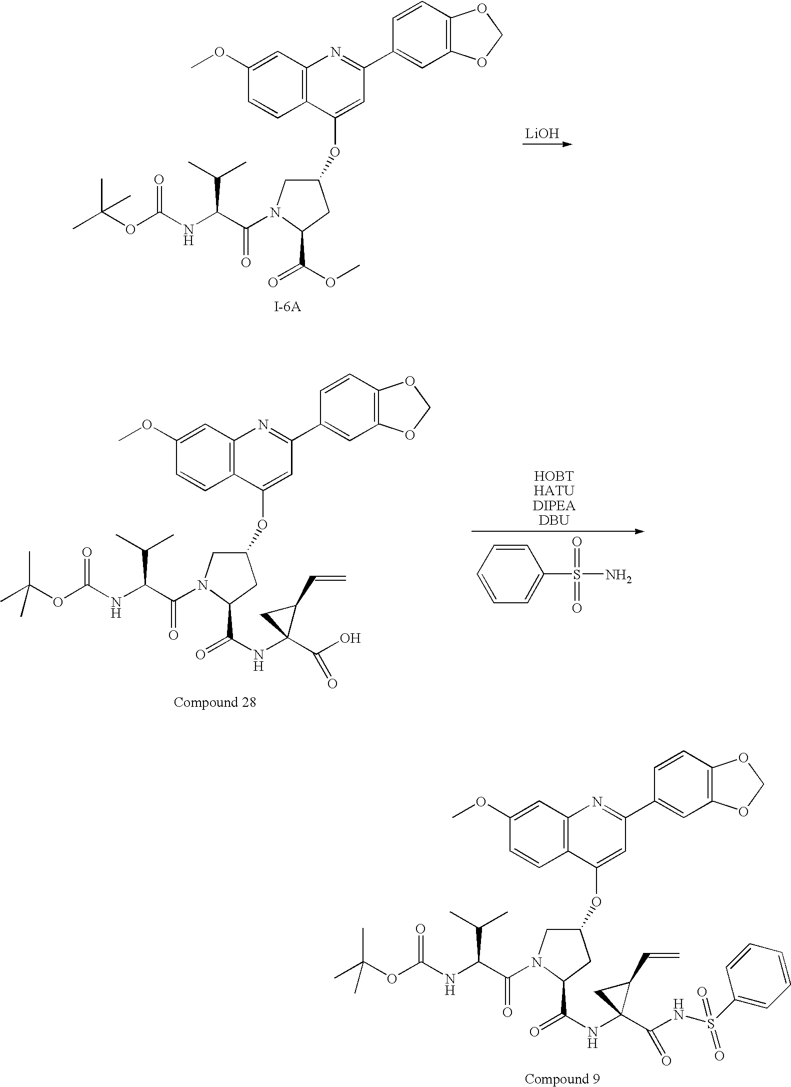

Oxalyl chloride (1.27 g, 10 mmol) was added to a solution of piperonylic acid (0.83 g, 5 mmol) in THF (20 mL) at 0° C. DMF (0.5 mL) was then slowly added to the reaction mixture. After the reaction mixture was stirred overnight at room temperature, THF and excess oxalyl chloride were removed under vacuum. The crude product was dissolved in dry pyridine (20 mL). 1-(2-amino-4-methoxyphenyl)ethanone (0.91 g, 5.0 mmol) was added to the mixture and stirred at 0° C. for 2.5 hours. The reaction mixture was allowed to slowly warm to room temperature and stirred overnight. After pyridine was removed, the residue was poured into ice water and quenched with an 1N HCl aqueous solution to adjust pH to 7. After the mixture was extracted with CH₂Cl₂, the organic layer was washed with a saturated NaHCO₃ aqueous solution, dried over anhydrous MgSO₄, filtered, and concentrated under vacuum. The crude product I-1A was obtained (1.5 g, 95%) and was used in the next step without further purification. ESI-MS (M+H⁺)=314.

t-BuOK (2.2 g, 20.0 mmol, 4 eq.) was added to a suspension of compound I-1A (1.5 g, 5.0 mmol) in anhydrous t-BuOH (40 mL). After the reaction mixture was heated under reflux for 2 hours, it was then cooled to room temperature and acidified by adding HCl (4N in dioxane, 1.5 eq.). The mixture was then concentrated under vacuum to give crude compound I-2A, which was used in the next step without further purification. ESI-MS (M+H⁺)=296.

A solution of compound I-2A (5.0 mmol) in POCl₃ (5.0 mL, 55 mmol) was heated to reflux for 1.5 hours. After removal of POCl₃ in vacuum, the residue was quenched by 2N NaOH to pH>7, stirred for 15 minutes, and then extracted by CH₂Cl₂.

The organic layer was dried over anhydrous MgSO₄, filtered, and concentrated under vacuum. The crude product thus obtained was then purified by silica gel column chromatography to give compound I-3A (1.1 g, 72%). ESI-MS (M+H⁺)=314.

To a suspension of Boc-4R-hydroxyproline (1.48 g, 6.4 mmol) in DMSO (20 mL) was added t-BuOK (2.15 g, 19.2 mmol) at 0° C. The mixture was then allowed to return to room temperature. After the reaction mixture was stirred for 1 hour, compound I-3A (2.67 g, 8.2 mmol) was added in three portions over 1 hour. The reaction mixture was stirred for 1 day and then was poured into cold water. The aqueous solution was acidified to pH 4.6 and filtered to give crude compound I-4A, which was used in the next step without further purification. ESI-MS (M+H⁺)=509.

To a solution of crude compound I-4A (0.3 g, 0.6 mmol) in MeOH (6 mL) was added SOCl₂ (0.1 g, 1.2 mmol) at room temperature. The reaction mixture was heated to reflux for 1 hour. MeOH and SOCl₂ were removed to give crude compound I-5A, which was used in the next step without further purification. ESI-MS (M+H⁺)=423.

N-Methylmorpholine (NMM, 0.2 g, 1.8 mmol) was added to a solution of crude compound I-5A (0.28 g, 0.6 mmol), 2-(1H-7-Azabenzotriazol-1-yl)-1,1,3,3-tetramethyl uronium hexafluoro-phosphate methanaminium (HATU, 0.45 g, 1.2 mmol), N-Hydroxybenzotriazole (HOBT, 0.04 g, 0.3 mmol), and Boc-VAL-OH (0.13 g, 0.6 mmol) in CH₂Cl₂ (5.0 mL) at room temperature. After the mixture was stirred overnight, it was concentrated under vacuum. The residue thus obtained was purified by silica gel column chromatography to give compound I-6A (0.28 g, 75%). ESI-MS (M+H⁺)=622.

To a solution of I-6A (0.28 g, 0.45 mmol) in THF (5.0 mL) was added 0.5 M LiOH (0.45 mL, 0.9 mmol) at room temperature. After the reaction mixture was stirred overnight, it was acidified by 10% HCl to pH<7 and concentrated under vacuum to give a yellow solid. The solid was washed by water and filtered to give compound I-7A. ESI-MS (M+H⁺)=608.

NMM (0.1 g, 1.0 mmol) was added to a solution of compound I-7A (0.2 g, 0.33 mmol), HATU (0.25 g, 0.66 mmol), HOBT (0.03 g, 0.17 mmol), and ethyl 1-amino-2-vinylcyclopropanecarboxylate (0.076 g, 0.41 mmol) in CH₂Cl₂ (10 mL) at room temperature. After the reaction mixture was stirred overnight, it was concentrated under vacuum. The residue was purified by silica gel column chromatography to give compound I-8A (0.17 g, 70%). ESI-MS (M+H⁺)=745.

To a solution of I-8A (0.17 g, 0.23 mmol) in THF (3.0 mL) was added 0.5 M LiOH (0.4 mL, 0.8 mmol) at room temperature. After the reaction mixture was stirred overnight, it was acidified by 10% HCl to pH<7 and concentrated under vacuum to give a yellow solid. The solid was washed by water and filtered to give Compound 28. ESI-MS (M+H⁺)=717.

A solution of Compound 28 (0.17 g, 0.24 mmol), HATU (0.18 g, 0.47 mmol) and 4-dimethylaminopyridine (DMAP, 0.14 g, 1.17 mmol) in CH₂Cl₂ (5 mL) was stirred at room temperature for 1 hour, followed by dropwise addition of benzenesulfonamide (0.073 g, 0.47 mmol), diisopropylethylamine (DIPEA, 0.18 mL, 1.4 mmol) and 1,8-diazabicyclo[5,4,0]undec-7-ene (DBU, 0.18 g, 1.17 mmol) over a period of 15 minutes. After the reaction mixture was stirred at room temperature overnight, the solvent was removed by vacuum. The residue thus obtained was purified by silica gel column chromatography to give Compound 9 (22 mg, 11%). ¹H NMR (CDCl₃) δ 7.93 (d, J=6.9 Hz, 2H), 7.82 (d, J=9.3 Hz, 1H), 7.58-7.35 (m, 6H), 6.96-6.85 (s, 3H), 6.04 (s, 2H), 5.77-5.69 (m, 1H), 5.30 (d, J=6.1 Hz, 1H), 5.04 (d, J=17.4 Hz, 1H), 4.76 (d, J=10.2 Hz, 1H), 4.60-4.55 (m, 1H), 4.21-4.18 (m, 1H), 4.04 (d, J=11.2 Hz, 1H), 3.95 (s, 3H), 3.77 (t, J=6.6 Hz, 1H), 2.64 (m, 1H), 2.50-2.20 (m, 4H), 2.18 (m, 1H), 1.38 (d, J=6.3 Hz, 3H), 1.07 (s, 9H), 0.48 (d, J=3.2 Hz, 3H). ESI-MS (M+H⁺)=856.

### EXAMPLE 10

### Preparation of Compound 10

Compound 10 was prepared (0.15 g, 84%) in a manner similar to that described in Example 1. ¹H NMR (CD₃OD+CDCl₃) δ 8.51 (d, J=7.5 Hz, 1H), 8.13 (d, J=9.1 Hz, 1H), 7.96 (s, 1H), 7.84-7.80 (m, 2H), 7.19 (d, J=9.6 Hz, 1H), 5.77-5.68 (m, 2H), 5.19 (d, J=17.1 Hz, 1H), 5.02 (m, 1H), 4.67-4.62 (m, 2H), 4.41 (m, 1H), 4.11-4.04 (m, 2H), 3.96 (s, 3H), 3.09 (t, J=7.2 Hz, 2H), 3.01 (t, J=7.2 Hz, 2H), 2.75-2.69 (m, 1H), 2.60-2.57 (m, 1H), 2.20 (t, J=3.6 Hz, 3H), 1.94 (m, 1H), 1.71 (t, J=6.6 Hz, 2H), 1.69-1.30 (m, 8H), 0.86 (d, J=6.6 Hz, 3H), 0.77 (d, J=6.9 Hz, 3H). ESI-MS (M+H⁺)=726.

### EXAMPLE 11

### Preparation of Compound 11

Compound 11 was prepared (19 mg, 11%) in a manner similar to that described in Example 2. ¹H NMR (CDCl₃) δ 8.36 (d, J=7.8 Hz, 1H), 8.03-7.98 (m, 2H), 7.82 (s, 1H), 7.66 (d, J=8.1 Hz, 1H), 7.57 (d, J=7.2 Hz, 1H), 7.50-7.42 (m, 3H), 7.08-7.04 (m, 2H), 5.59-5.56 (m, 2H), 5.12 (d, J=17.2 Hz, 1H), 4.96 (d, J=9.0 Hz, 2H), 4.55-4.44 (m, 2H), 4.29-4.23 (m, 1H), 4.13-4.10 (m, 1H), 3.96 (s, 3H), 3.05 (t, J=7.5 Hz, 2H), 3.00 (t, J=7.5 Hz, 2H), 2.65 (m, 1H), 2.53 (m, 1H), 2.17 (m, 3H), 2.09-1.52 (m, 11H), 1.01-0.88 (m, 6H). ESI-MS (M+H⁺)=864.

### EXAMPLE 12

### Preparation of Compound 12

Compound 12 was prepared (0.15 g, 76%) in a manner similar to that described in Example 1. ¹H NMR (CD₃OD+CDCl₃) δ 8.28-8.22 (m, 1H), 8.10 (d, J=8.7 Hz, 1H), 7.72 (d, J=4.2 Hz, 2H), 7.64 (d, J=7.5 Hz, 1H), 7.15 (d, J=9.2 Hz, 1H), 5.69-5.60 (m, 2H), 5.13 (d, J=17.4 Hz, 1H), 4.97 (d, J=9.2 Hz, 1H), 4.58 (s, 2H), 4.35 (d, J=11.6 Hz, 1H), 4.20-4.01 (m, 2H), 3.96 (s, 3H), 2.96 (m, 2H), 2.80 (m, 2H), 2.62-2.48 (m, 2H), 2.07-1.98 (m, 1H), 1.90-1.70 (m, 4H), 1.67-1.22 (m, 11H), 0.84-0.73 (m, 6H). ESI-MS (M+H⁺)=740.

### EXAMPLE 13

### Preparation of Compound 13

Compound 13 was prepared (24 mg, 12%) in a manner similar to that described in Example 2. ¹H NMR (CDCl₃) δ 8.29 (d, J=7.8 Hz, 1H), 8.02-7.97 (m, 2H), 7.81 (s, 1H), 7.53 (m, 5H), 7.16 (s, 1H), 7.02 (d, J=8.6 Hz, 1H), 5.67 (d, J=9.0 Hz, 1H), 5.54 (s, 1H), 5.13 (d, J=17.2 Hz, 1H), 4.97-4.94 (m, 2H), 4.55-4.51 (m, 2H), 4.26 (t, J=8.4 Hz, 1H), 4.12 (d, J=8.7 Hz, 1H), 3.96 (s, 3H), 3.00 (m, 2H), 2.84 (m, 2H), 2.64 (m, 1H), 2.50 (m, 1H), 2.17 (m, 1H), 2.09-1.20 (m, 15H), 1.02-0.95 (m, 6H). ESI-MS (M+H⁺)=879.

### EXAMPLE 14

### Preparation of Compound 14

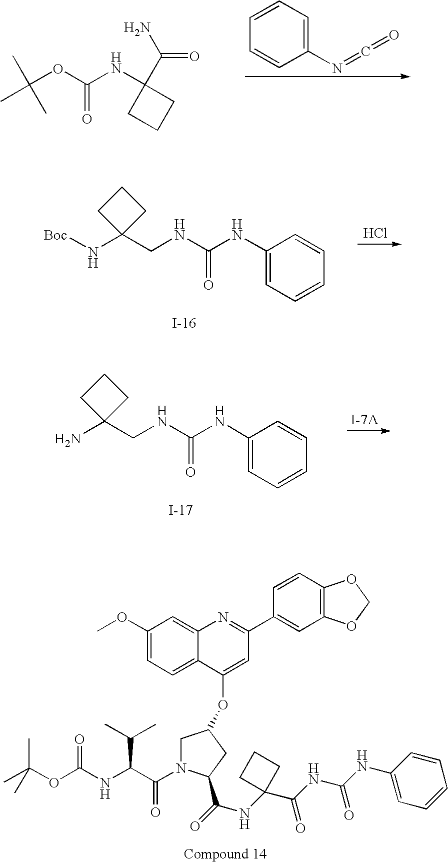

Phenyl isocyanate (0.25 g, 2.1 mmol) was added to a solution of tert-butyl 1-carbamoylcyclobutylcarbamate (0.3 g, 1.4 mmol) in toluene (20 ml) at room temperature. After the reaction mixture was refluxed overnight, it was concentrated under vacuum. The crude product thus obtained was purified by silica gel column chromatography to give compound I-16 (0.14 g, 29%). ESI-MS (M+H⁺)=333.

To a solution of compound I-16 (0.14 g, 0.42 mmol) in CH₂Cl₂ was added 4 M HCl in dioxane (1.1 mL, 4.2 mmol) at room temperature. After the reaction mixture was stirred overnight, it was concentrated under vacuum to give compound I-17 (0.11 g, 95%). ESI-MS (M+H⁺)=234.

Compound 14 was prepared (80 mg, 65%) in a manner similar to that described in Example 1 except that ethyl 1-amino-2-vinylcyclopropanecarboxylate used in the preparation of compound I-11 was replaced with compound I-17. ¹H NMR (CDCl₃) δ 8.06 (d, J=9.0 Hz, 1H), 7.50-7.39 (m, 5H), 7.30 (d, J=10.2 Hz, 2H), 7.07 (m, 2H), 6.89-6.85 (m, 2H), 5.99 (d, J=5.4 Hz, 2H), 5.70 (d, J=8.1 Hz, 1H), 5.39 (s, 1H), 4.76 (t, 1H), 4.63 (d, J=11.6 Hz, 1H), 4.07-4.01 (m, 2H), 3.98 (s, 3H), 3.09-3.03 (m, 1H), 2.60-2.41 (m, 2H), 2.39-2.23 (m, 2H), 2.09-1.92 (m, 4H), 1.20 (s, 9H), 0.92-0.77 (m, 6H). ESI-MS (M+H⁺)=823.

### EXAMPLE 15

### Preparation of Compound 15

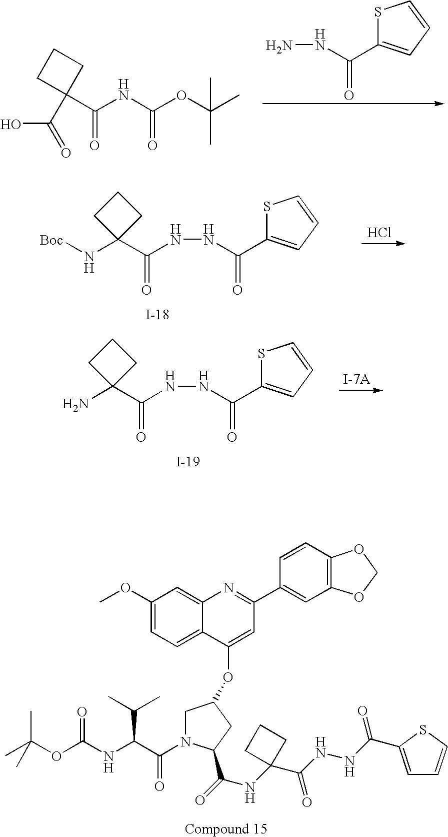

CDI (0.45 g, 2.8 mmol) was added to a solution of 1-(tert-butoxycarbonyl)-cyclobutane carboxylic acid (0.3 g, 1.4 mmol) and 2-(thiophene-2-carbonyl)hydrazine carboxylic acid (0.3 g, 2.1 mmol) in THF at room temperature. After the reaction mixture was refluxed for 3 days, it was concentrated under vacuum. The residue thus obtained was purified by silica gel column chromatography to give compound I-18 (0.3 g, 63%). ESI-MS (M+H⁺)=340.

Compound I-19 was prepared (0.13 g, 90%) in a manner similar to that described for the preparation of compound I-17. ESI-MS (M+H⁺)=240.

Compound 15 was prepared (135 mg, 62%) in a manner similar to that described in Example 1 except that ethyl 1-amino-2-vinylcyclopropanecarboxylate used in the preparation of compound I-11 was replaced with compound I-19. ¹H NMR (CDCl₃) δ 7.90 (d, J=8.4 Hz, 1H), 7.60-7.40 (m, 4H), 7.29 (s, 1H), 7.03-6.91 (m, 4H), 6.01 (s, 2H), 5.73 (s, 1H), 5.38 (s, 1H), 4.68 (m, 1H), 4.59 (d, J=11.2 Hz, 1H), 4.19 (t, J=8.8 Hz, 1H), 4.07 (d, J=12.3 Hz, 1H), 3.97 (s, 3H), 2.94 (m, 1H), 2.70-2.63 (m, 3H), 2.23-2.15 (m, 2H), 2.04-2.00 (m, 3H), 1.33 (s, 9H), 0.91 (m, 3H), 0.88 (m, 3H). ESI-MS (M+H⁺)=829.

### EXAMPLE 16

### Preparation of Compound 16

Compound 16 was prepared (0.15 g, 82%) in a manner similar to that described in Example 20. ¹H NMR (CD₃OD+CDCl₃) δ 8.53 (s, 1H), 7.88 (d, J=9.2 Hz, 1H), 7.83 (s, 1H), 7.77 (d, J=8.1 Hz, 1H), 7.64 (s, 1H), 7.29 (d, J=9.3 Hz, 1H), 7.00 (d, J=7.8 Hz, 1H), 6.06 (s, 2H), 5.82-5.65 (m, 1H), 5.23 (d, J=15.8 Hz, 2H), 5.08-5.02 (m, 2H), 4.62 (m, 1H), 4.49 (s, 1H), 4.25-4.16 (m, 2H), 3.96 (s, 3H), 3.77 (m, 1H), 2.51-2.35 (m, 2H), 2.15-2.06 (m, 1H), 1.99-1.92 (m, 1H), 1.76 (t, J=6.8 Hz, 1H), 1.43-1.41 (m, 1H), 1.12 (s, 9H), 0.95 (d, J=6.8 Hz, 3H), 0.87 (d, J=6.6 Hz, 3H). ESI-MS (M+H⁺)=731.

### EXAMPLE 17

### Preparation of Compound 17

Compound 17 was prepared (21 mg, 12%) from compound 16 in a manner similar to that described in Example 21. ¹H NMR (CDCl₃) δ 7.97 (d, J=7.5 Hz, 2H), 7.79 (d, J=9.3 Hz, 1H), 7.68-7.42 (m, 7H), 7.15 (d, J=9.0 Hz, 1H), 6.92 (d, J=8.2 Hz, 1H), 6.03 (s, 2H), 5.51 (m, 1H), 5.11 (d, J=15.9 Hz, 2H), 4.91 (t, J=11.6 Hz, 2H), 4.43 (brs, 3H), 4.25 (t, J=8.6 Hz, 1H), 3.96 (s, 3H), 3.77 (d, J=8.4 Hz, 1H), 2.41-2.35 (m, 1H), 2.26-2.01 (m, 4H), 1.80 (t, J=6.6 Hz, 1H), 1.30 (s, 9H), 0.96 (m, 3H), 0.94 (m, 3H). ESI-MS (M+H⁺)=870.

### EXAMPLE 18

### Preparation of Compound 18

A mixed solution of cyclopentanol (10.9 mL, 120 mmol) and pyridine (10.7 mL, 132 mmol) was dripped into a solution of triphosgene (11.87 g, 40.0 mmol) in anhydrous diethyl ether (100 mL) under −70° C. After the mixture was stirred for 1 hour, it was removed from the low temperature bath. After a further stirring of 1.5 hours, 1.0 N HCl_{(aq)} (110 mL) was added. The organic phase was separated and washed with brine (60 mL) and dried with Na₂SO₄. The solvent was removed by distillation under reduced pressure to give cyclopentyl chloroformate (14.395 g, bp. 80° C. at 75 mm-Hg, 80.7%). The cyclopentyl chloroformate was used in the next step without further purification.

To a stirred and cooled (0° C.) solution of the L-valine methyl ester hydrochloride (13.528 g, 80.7 mmol) in H₂O (80.7 mmol) were added dropwise a 1 M Na₂CO₃ aqueous solution (89 mL) and cyclopentyl chloroformate (14.395 g, 96.9 mmol). The ice-bath was then removed and the reaction mixture was stirred at room temperature for 3 hours. The organic layer was subsequently extracted with diethyl ether (3×160 mL), dried with anhydrous MgSO₄, and concentrated to give crude cyclopentyloxycarbonyl-L-valine methyl ester, which was used in the next reaction without further purification.

1.0 N sodium hydroxide_{(aq)} (97 mL) was added to a solution of cyclopentyloxy-carbonyl-L-valine methyl ester (19.634 g, 80.7 mmol) in CH₃OH (403.5 mL) at room temperature. The resulting suspension was stirred at room temperature for 4 hours and the was partitioned between 10% KHSO_{4(aq)} (132 mL) and CH₂Cl₂ (3×418 mL). The combined organic layers were dried over Na₂SO₄ and concentrated under reduced pressure. The crude product thus obtained was purified by flash chromatography using 6% CH₃OH in CH₂Cl₂ as the eluent to give (S)-2-(cyclopentyloxycarbonylamino)-3-methylbutanoic acid as a oily syrup (17.968 g). ESI-MS (M+H⁺)=230.

Compound 18 was prepared (0.13 g, 76%) in a manner similar to that described in Example 20 except that Boc-VAL-OH used in the preparation of compound I-25 was replaced with (S)-2-(cyclopentyloxycarbonyl-amino)-3-methylbutanoic acid obtained above. ¹H NMR (CD₃OD+CDCl₃) δ 8.58 (s, 1H), 7.89 (d, J=9.0 Hz, 1H), 7.85 (s, 1H), 7.80 (d, J=8.4 Hz, 1H), 7.66 (s, 1H), 7.31 (d, J=9.3 Hz, 1H), 7.03 (d, J=8.1 Hz, 1H), 6.07 (s, 2H), 5.74-5.71 (m, 1H), 5.22 (d, J=16.8 Hz, 2H), 4.97-4.87 (m, 2H), 4.72 (m, 1H), 4.59 (t, J=7.1 Hz, 1H), 4.49 (s, 1H), 4.25-4.18 (m, 2H), 3.96 (s, 3H), 3.77 (d, J=9.6 Hz, 1H), 2.49-2.43 (m, 1H), 2.37-2.33 (m, 1H), 2.15-2.06 (m, 1H), 1.99-1.92 (m, 1H), 1.76-1.72 (m, 1H), 2.39-1.18 (m, 9H), 0.94 (d, J=6.9 Hz, 3H), 0.90 (d, J=6.6 Hz, 3H). ESI-MS (M+H⁺)=743.

### EXAMPLE 19

### Preparation of Compound 19

Compound 19 was prepared (19 mg, 11%) from compound 18 in a manner similar to that described in Example 21. ¹H NMR (CDCl₃) δ 7.98 (d, J=7.5 Hz, 2H), 7.81 (d, J=9.0 Hz, 1H), 7.71-7.75 (m, 7H), 7.15 (m, 1H), 6.94 (d, J=8.1 Hz, 1H), 6.03 (s, 2H), 5.53-5.50 (m, 1H), 5.13 (d, J=14.4 Hz, 2H), 4.95 (d, J=9.8 Hz, 2H), 4.87 (brs, 1H), 4.44 (brs, 3H), 4.27 (t, J=8.6 Hz, 1H), 3.96 (s, 3H), 3.78 (d, J=8.7 Hz, 1H), 2.39-2.00 (m, 4H), 1.95-1.18 (m, 10H), 0.95 (m, 3H), 0.92 (m, 3H). ESI-MS (M+H⁺)=882.

### EXAMPLE 20

### Preparation of Compound 20

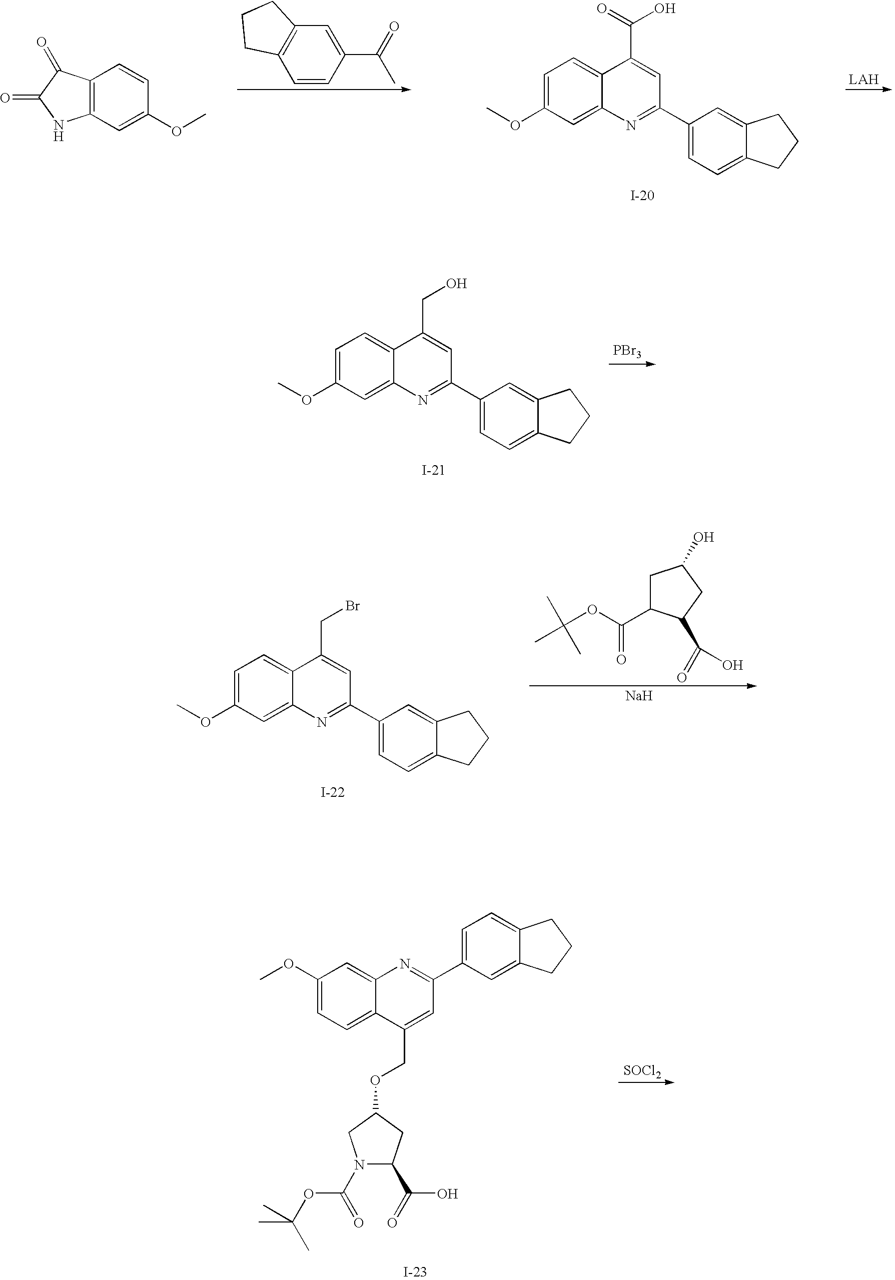

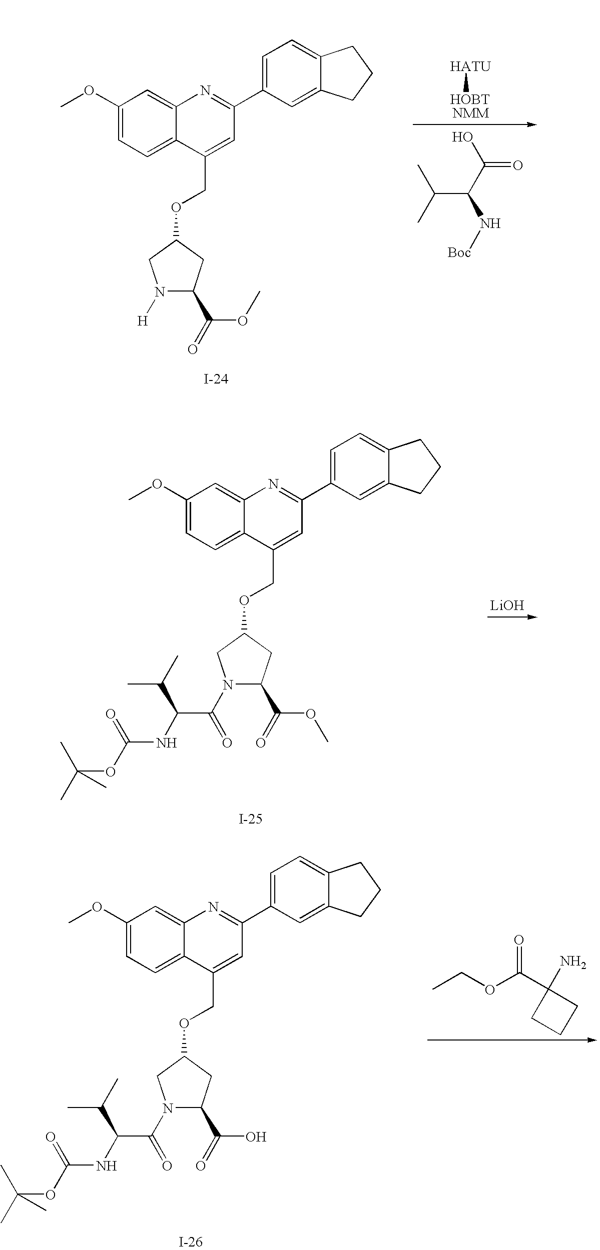

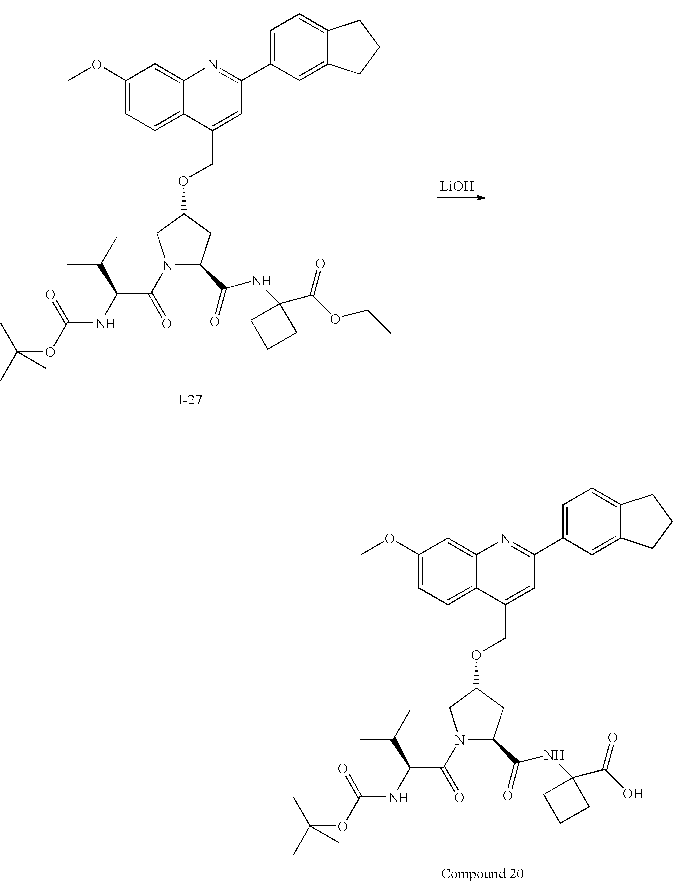

6-Methoxylisatin (6 g, 33.87 mmol), 1-indan-ethanone (40.64 mmol), and 85% KOH pellets (120 mmol) were dissolved in EtOH (40 mL). The reaction mixture was stirred at 80° C. for 24 hours. After the solvent was removed by evaporation, the residue thus obtained was dissolved in H₂O (50 mL). The solution was then washed twice with Et₂O (30 mL). The aqueous phase was cooled by an ice-water bath and acidified with a 37% HCl aqueous solution to pH 1. The precipitate was collected by suction filtration, washed with H₂O, and dried to give compound I-20, 2-indan-5-yl-7-methoxy-quinoline-4-carboxylic acid (9.5 g, 88%) as a solid. ESI-MS (M+H⁺)=320.

To a stirred suspension of compound I-20 (1 g, 1 eq.) in THF (30 mL) was added slowly LiAlH₄ (LAH, 1 g, 8 eq.) at −10° C. under a dry nitrogen atmosphere. The mixture was stirred at −5° C. for 20 hours. The reaction was quenched with cold H₂O (20 mL). After evaporation of THF, ethyl acetate (15 mL) was added and the mixture was filtered. The organic layer were separated, dried over anhydrous Na₂SO₄, concentrated, and dried to give compound I-21, (2-indan-5-yl-7-methoxy-quinolin-4-yl)-methanol (0.8 g, 83%) as an oil. ESI-MS (M+H⁺)=306.

To a solution of compound I-21 (5 g, 1 eq.) in THF (100 mL) was added dropwise PBr₃ (2.2 eq) at 0° C. The resulting mixture was stirred at 0° C. for 1 hour and then slowly warmed to room temperature. After the mixture was stirred for 16 hours, the reaction was quenched with a NaHCO₃(sat) aqueous solution at 0° C. and the PH was adjusted to above 7. The mixture was then extracted with CH₂Cl₂. The organic layer was separated, dried, and concentrated to give a brown oil. The crude compound was purified by silica gel column chromatography to give compound I-22, 4-bromomethyl-2-indan-5-yl-7-methoxy-quinoline (3.6 g, 60%) as a solid. ESI-MS (M+H⁺)=370.

NaH (0.4 g 60%) was added to a solution of N-Boc-4-hydroxyproline carboxylic acid (0.75 g 1.2 eq.) in THF (10 mL). The mixture was stirred at room temperature for 1 hour. A solution of compound I-22 (1 g, 2.7 mmol) in THF (10 mL) was slowly added to the mixture at room temperature within 5˜10 minutes. After 10 minutes, the mixture was heated at 80° C. for 20 hour. After the mixture was allowed to cool down to room temperature, H₂O (15 mL) was added. The mixture was then extracted with ethyl acetate. The organic layer was separated, dried, and concentrated to afford a crude oil, which was purified by silica gel column chromatography to give compound I-23 (1.12 g, 80%) as a solid. ESI-MS (M+H⁺)=519.

SOCl₂ (2.2 eq.) was added to a solution of compound I-23 (1.20 g, 1 eq.) in dry methanol (10 mL). After the mixture was heated at 65° C. for 1.5 hours, the solvent was evaporated and the residue was dried under high vacuum to give the HCl salt of compound I-24, which was used in next step without any purification.

To a stirred suspension of the crude compound I-24 (1.0 g, 1 eq.) in CH₂Cl₂ (25 mL) were sequentially added Boc-VAL-OH (0.5 g, 1.0 eq.), HOBT (0.16 g, 0.5 eq.), NMM (0.7 g, 3 eq.) and HATU (1.76 g, 2 eq.). The mixture was stirred at room temperature for 16 hours. After the CH₂Cl₂ was evaporated, the residue was added into EtOAc (50 mL). The organic solution was washed with a 10% NaHCO₃ aqueous solution (40 mL), dried, and concentrated. The crude product obtained was purified by silica gel column chromatography on silica gel to give compound I-25 (1.34 g, 92%) as an oil. ESI-MS (M+H⁺)=632.

0.5 M LiOH_{(aq)} (4 mL, 4 eq.) was added to a solution of compound I-25 (1 g, 1 eq.) obtained in THF (20 mL). The solution was stirred at room temperature for 20 hours, and was acidified with a 10% HCl aqueous solution to PH˜3. After the solvents were removed under vacuum, the resulting residue was washed with H₂O to give compound I-26 (0.86 g, 88%) as a solid. ESI-MS (M+H⁺)=618.

To a stirred suspension of crude compound I-26 (0.15 g, 1 eq.) in CH₂Cl₂ (5 mL) were sequentially added ethyl 1-amino-1-cyclobutanecarboxylate monohydrochloride (0.05 g, 1.0 eq.), HOBT (0.02 g, 0.5 eq.), NMM (0.08 g, 3 eq.), and HATU (0.20 g, 2 eq.). The mixture was stirred at room temperature for 16 hours. After the CH₂Cl₂ was evaporated, the residue was added into EtOAc (10 mL). The organic solution was washed with a 10% NaHCO₃ aqueous solution (10 mL), dried, and concentrated. The crude product thus obtained was purified by silica gel column chromatography on silica gel to give compound I-27 (0.15 g, 83%) as an oil. ESI-MS (M+H⁺)=743.

0.5M LiOH_{(aq)} (1.2 ml, 4 eq) was added in a solution of compound I-27 (0.15 g, 1 eq.) in THF/MeOH (5/5 mL). The solution was stirred at room temperature for 20 hours, and was then acidified with a 10% HCl aqueous solution to PH˜3. After the solvents were removed under vacuum, the resulting residue was washed with H₂O to give compound 20 (0.11 g, 76%) as a solid. ¹H NMR (CDCl₃) δ 8.08-8.01 (m, 2H), 7.89-7.81 (m, 3H), 7.47-7.44 (d, J=7.5 Hz, 1H), 7.26 (s, 1H), 5.28-5.09 (m, 3H), 4.85-4.82 (dd, J=17.1 Hz, 7.2 Hz, 1H), 4.51 (brs, 1H), 4.34-4.30 (d, J=11.7 Hz, 1H), 4.26-4.20 (dd, J=17.7 Hz, 7.8 Hz, 1H), 4.05 (s, 3H), 3.77-3.74 (m, 1H), 3.08-3.03 (m, 2H), 3.00-2.94 (m, 2H), 2.76-2.59 (m, 3H), 2.45-2.40 (m, 1H), 2.30-2.21 (m, 2H), 2.16-2.03 (m, 2H), 2.06 (m, 3H), 1.22 (s, 9H), 0.98 (d, J=6.6 Hz, 3H), 0.91 (d, J=6.6 Hz, 3H). ESI-MS (M+H⁺)=715.

### EXAMPLE 21

### Preparation of Compound 21

HATU (0.12 g, 0.32 mmol) was added to a solution of compound 20 (0.11 g, 0.15 mmol) and DIPEA (0.11 g, 0.82 mmol) in CH₂Cl₂ (6 mL) at room temperature. After the solution was stirred for 1 hour, DMAP (0.08 g, 0.67 mmol) and benzenesulfonamide (0.06 g, 0.39 mmol) were added. After the mixture was stirred for 15 minutes, DBU (0.12 g) was added dropwise. The resulting solution was stirred for 16 hours at room temperature and was then added into EtOAc (10 mL). The organic solution was washed with a 10% NaHCO₃ aqueous solution (10 mL), dried, and concentrated. The crude product thus obtained was purified by silica gel column chromatography on silica gel to give compound 21 (0.08 g, 60%) as a solid. ¹H NMR (CDCl₃) δ 8.04-8.01 (m, 3H), 7.95-7.83 (m, 3H), 7.61-7.37 (m, 5H), 7.21-7.17 (m, 1H), 5.26-5.22 (m, 1H), 5.18-5.13 (d, J=13.1 Hz, 1H), 5.00-4.96 (m, 1H), 4.58-4.53 (m, 1H), 4.42-4.33 (m, 2H), 4.26-4.20 (dd, J=10.1 Hz, 8.1 Hz, 1H), 3.98 (s, 3H), 3.75-3.72 (m, 1H), 3.06-2.93 (m, 4H), 2.73-2.62 (m, 1H), 2.58-2.51 (m, 1H), 2.43-2.31 (m, 2H), 2.17-2.12 (m, 2H), 2.09-2.03 (m, 3H), 2.03-1.93 (m, 2H), 1.34 (s, 9H), 0.97-0.94 (m, 6H). ESI-MS (M+H⁺)=854.

### EXAMPLE 22

### Preparation of Compound 22

Compound 22 was prepared in a manner similar to that described in Example 20 except that ethyl 1-amino-1-cyclobutanecarboxylate monohydrochloride used in preparing compound I-27 was replaced with ethyl 1-amino-2-vinylcyclopropane-carboxylate. ¹H NMR (CDCl₃) δ 8.29 (brs, 1H), 7.99-7.97 (d, J=8.7 Hz, 1H), 7.87-7.77 (m, 3H), 7.34-7.25 (m, 3H), 5.76-5.67 (m, 1H), 5.28-5.23 (m, 2H), 5.03-4.98 (m, 2H), 4.69-4.68 (m, 1H), 4.65-4.52 (dd, J=10.2 Hz, 6.4 Hz, 1H), 4.52-4.44 (m, 2H), 4.26-4.19 (dm, J=10.4 Hz, 1H), 4.05 (s, 3H), 3.81-3.79 (m, 1H), 2.91-2.90 (m, 2H), 2.69-2.59 (m, 3H), 2.41-2.35 (m, 1H), 2.34-2.16 (m, 1H), 2.09-1.81 (m, 4H), 1.80-1.49 (m, 8H), 1.48-1.41 (m, 3H), 0.98 (d, J=7.2 Hz, 3H), 0.96 (d, J=7.2 Hz, 3H). ESI-MS (M+H⁺)=739

### EXAMPLE 23

### Preparation of Compound 23

Compound 23 was prepared in a manner similar to that described in Example 21 using compound 22 as a starting material. ¹H NMR (CDCl₃) δ 8.01-7.97 (m, 2H), 7.86-7.84 (m, 2H), 7.72 (brs, 1H), 7.52-7.46 (m, 4H), 7.37-7.34 (m, 1H), 7.19-7.16 (m, 1H), 7.10-7.09 (m, 1H), 5.59-5.48 (m, 1H), 5.16-5.12 (m, 2H), 4.97-4.89 (m, 3H), 4.40-4.28 (m, 4H), 3.97 (s, 3H), 3.78-3.74 (m, 1H), 3.03-2.99 (m, 4H), 2.29-2.22 (m, 1H), 2.22-2.04 (m, 2H), 2.04-2.00 (m, 1H), 1.82-1.24 (m, 12H), 0.96 (m, 6H). ESI-MS (M+H⁺)=878.

### EXAMPLE 24

### Preparation of Compound 24

Compound 24 was prepared in a manner similar to that described in Example 1 by using compound I-30 as a starting material. ¹H NMR (CDCl₃) δ 8.10-8.01 (m, 2H), 7.60-7.58 (m, 2H), 7.19-7.13 (m, 3H), 5.67-5.58 (m, 3H), 5.17-5.11 (m, 1H), 5.00-4.96 (m, 1H), 4.59-4.56 (m, 1H), 4.40-4.35 (m, 1H), 4.02-3.99 (m, 2H), 3.96 (s, 3H), 2.78-2.71 (m, 4H), 2.70-2.58 (m, 1H), 2.58-2.42 (m, 1H), 2.06-2.02 (m, 1H), 1.88-1.80 (m, 1H), 1.72-1.63 (m, 6H), 1.19 (s, 9H), 0.85 (d, J=5.7 Hz, 3H), 0.83 (d, J=5.7 Hz, 3H). ESI-MS (M+H⁺)=727.

Compound I-30 was prepared as follows:

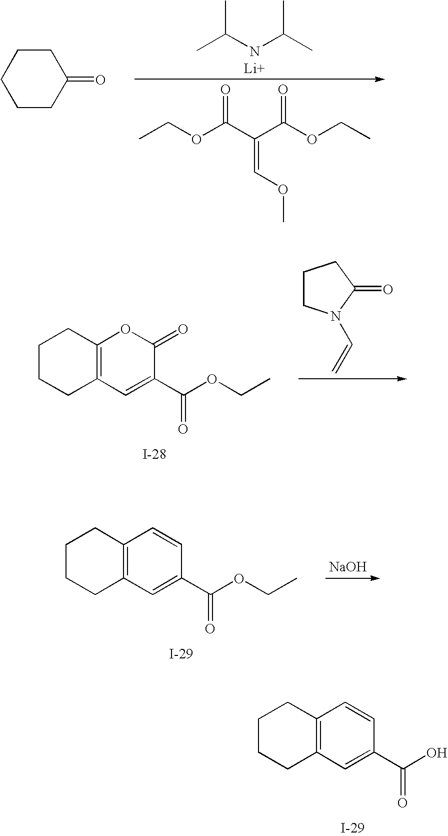

A solution of cyclohexanone (5 g, 1 eq.) in 60 mL of dry THF was added dropwise (over a period of 30 minutes) to a −78° C. solution of freshly generated lithium diisopropylamide (31 mL, 1.2 eq.) in 75 mL of dry THF under dry N₂. The resulting mixture was stirred at −78° C. to −5° C. over 2 hours. After the solution was cooled to −30° C., diethyl ethoxymethylene malonate (12.4 mL, 1.2 eq.) in 20 mL of THF was added slowly (over a period of 15˜20 minutes). The mixture was stirred at −30° C. to 25° C. for 16 hours. The solution was then poured onto 100 mL of 5% aqueous HCl and extracted with CH₂Cl₂ (5×30 mL). The combined extracts were dried over Na₂SO₄ and concentrated under vacuum. The crude product thus obtained was purified by silica gel column chromatography to give compound I-28 as a white solid (5.7 g, 50%). ¹H NMR (CDCl₃) δ.7.98 (s, 1H), 4.35 (q, J=6.9 Hz, 2H), 2.60-2.55 (m, 2H), 2.48-2.44 (m, 2H), 1.84-1.74 (m, 4H), 1.36 (t, J=7.2 Hz, 3H). ESI-MS (M+H⁺)=223.

A solution containing compound I-28 (0.5 g, 1 eq.) and N-vinyl-2-pyrrolidone (0.75 g, 3 eq.) in 2.0 mL of dry mesitylene was heated at 160° C. (bath temperature) for 48 hours in a sealed vessel. The mixture was then cooled, concentrated under vacuum, and purified by silica gel column chromatography to give compound I-29 as a white solid (0.37 g, 80%). ¹H NMR (CDCl₃) δ.7.75-7.73 (m, 2H), 7.12-7.09 (d, J=8.7 Hz, 1H), 4.35 (q, J=6.9 Hz, 2H), 2.81 (m, 4H), 1.81 (m, 4H), 1.38 (t, J=7.5 Hz, 3H). ESI-MS (M+H⁺)=205.

A solution of compound I-29 (5.6 g, 1 eq.) in THF (70 mL) was treated with a 1 N aqueous NaOH solution (80 mL). The resulting mixture was warmed at 60° C. for 20 hours, and then diluted with diethyl ether. After the mixture was extracted twice with a saturated aqueous NaHCO₃ solution, 10% HCl was added to neutralize the mixture to PH=3˜5. The aqueous layer was then extracted with diethyl ether. The combined diethyl ether layer was dried over Na₂SO₄, filtered, and concentrated to give compound I-30 (4.66 g, 95%). ¹H NMR (CDCl₃) δ.7.81-7.78 (m, 2H), 7.16-7.13 (d, J=7.5 Hz, 1H), 2.82 (m, 4H), 1.84-1.80 (m, 4H). ESI-MS (M+H⁺)=177.

### EXAMPLE 25

### Preparation of Compound 25

Compound 25 was prepared in a manner similar to that described in Example 2 using compound 24 as a starting material. ¹H NMR (CDCl₃) δ 8.00-7.97 (d, J=9.3 Hz, 2H), 7.76-7.69 (m, 3H), 7.50-7.43 (m, 2H), 7.20-7.17 (d, J=8.1 Hz, 2H), 7.08-6.93 (m, 3H), 5.89-5.82 (m, 1H), 5.37-5.31 (m, 2H), 5.29-5.07 (m, 3H), 4.51-4.47 (m, 1H), 4.28-4.19 (m, 1H), 4.15-4.05 (m, 1H), 3.95 (s, 3H), 2.89-2.77 (m, 4H), 2.77-2.74 (m, 1H), 2.57-2.52 (m, 1H), 2.16-1.99 (m, 2H), 1.84 (m, 4H), 1.79-1.76 (m, 2H), 1.35 (s, 9H), 1.01 (d J=6.6 Hz, 3H), 0.99 (d, J=6.6 Hz, 3H). ESI-MS (M+H⁺)=866.

### EXAMPLE 26

### Preparation of Compound 26

Compound 26 was prepared in a manner similar to that described Example 1 except that ethyl 1-amino-2-vinylcyclopropanecarboxylate used in preparing compound I-27 was replaced with compound I-15. ¹H NMR (CDCl₃) δ 8.13-8.05 (m, 2H), 7.61-7.53 (m, 3H), 7.29-7.12 (m, 2H), 6.89-6.58 (d, J=8.4 Hz, 1H), 6.47-6.44 (m, 1H), 5.77 (brs, 1H), 5.57 (m, 1H), 5.19-4.98 (m, 2H), 4.85-4.79 (m, 2H), 4.39-4.35 (m, 1H), 4.21-4.03 (m, 2H), 4.02 (s, 3H), 3.24-3.19 (m, 2H), 2.96-2.69 (m, 2H), 2.68 (m, 2H), 2.45 (m, 2H), 2.20-1.97 (m, 7H), 1.32 (s, 9H), 0.99-0.90 (m, 6H). ESI-MS (M+H⁺)=753.

### EXAMPLE 27

### Preparation of Compound 27

Compound 27 was prepared from compound 26 in a manner similar to that described for compound 2. ¹H NMR (CDCl₃) δ 8.04-7.94 (m, 3H), 7.74-7.68 (m, 2H), 7.61-7.42 (m, 4H), 7.20-7.16 (d, J=8.4 Hz, 1H), 7.05-6.94 (m, 1H), 6.86 (s, 1H), 6.43-6.37 (m, 1H), 5.53-5.37 (m, 2H), 5.29-5.25 (m, 1H), 5.07-4.88 (m, 3H), 4.57-4.49 (m, 1H), 4.20-4.02 (m, 2H), 3.95 (s, 3H), 3.38-3.24 (m, 1H), 3.00-2.82 (m, 6H), 2.71-2.42 (m, 3H), 2.15-2.10 (m, 1H), 1.84 (m, 4H), 1.40 (s, 9H), 1.11-0.98 (m, 6H). ESI-MS (M+H⁺)=892.

### EXAMPLE 28

### Preparation of Compound 28

Compound 28 was prepared in a manner similar to that described in Example 9. ¹H NMR (CDCl₃) δ 8.15-8.07 (m, 2H), 7.61-7.50 (m, 2H), 6.97-6.84 (m, 1H), 6.00 (s, 2H), 5.79-5.74 (m, 1H), 5.59-5.58 (m, 1H), 5.31-5.18 (m, 1H), 5.17-5.07 (m, 1H), 4.66-4.60 (m, 1H), 4.42-4.38 (m, 1H), 4.15-4.04 (m, 2H), 3.98 (s, 3H), 2.71-2.62 (m, 2H), 2.50-2.45 (m, 1H), 2.14-2.01 (m, 1H), 1.97-1.87 (m, 1H), 1.75-1.73 (m, 1H), 1.31 (s, 9H), 0.95-0.93 (m, 6H). ESI-MS (M+H⁺)=717.

### EXAMPLE 29

### Preparation of Compound 29

Compound 29 was prepared from compound 28 in a manner similar to that described in Example 25. ¹H NMR (CDCl₃) δ 8.03˜8.00 (d, J=7.8 Hz, 1H), 7.97-7.88 (m, 3H), 7.58-7.39 (m, 6H), 7.03-6.87 (m, 2H), 6.42-6.33 (m, 1H), 6.02 (s, 2H), 5.51-4.86 (m, 6H), 4.56-4.52 (m, 1H), 4.20-4.03 (m, 2H), 3.95 (s, 3H), 3.36-3.24 (m, 1H), 2.95-2.88 (m, 1H), 2.69-2.45 (m, 4H), 2.16-2.09 (m, 1H), 1.36 (s, 9H), 1.00-0.98 (m, 6H). ESI-MS (M+H⁺)=882.

### EXAMPLE 30

### Preparation of Compound 30

Compound 30 was prepared in a manner similar to that described in Example 24. ¹H NMR (CDCl₃) δ 8.17-8.10 (m, 2H), 7.87 (brs, 1H), 7.64-7.61 (d, J=8.4 Hz, 1H), 7.53 (s, 1H), 7.19-7.17 (m, 2H), 7.01-6.99 (d, J=8.1 Hz, 1H), 6.07 (s, 2H), 5.84-5.73 (m, 2H), 5.62 (brs, 1H), 5.26-5.20 (d, J=17.4 Hz, 1H), 5.09-5.05 (d, J=11.2 Hz, 1H), 4.77-4.76 (m, 1H), 4.68 (dd, J=10.2 Hz, 7.8 Hz, 1H), 4.44-4.40 (dm, J=11.7 Hz, 1H), 4.14-4.05 (m, 2H), 3.96 (s, 3H), 2.73-2.68 (m, 1H), 2.57-2.50 (m, 1H), 2.16-2.07 (m, 1H), 2.07-1.94 (m, 1H), 1.76-1.72 (m, 1H), 1.65-1.43 (m, 8H), 1.43-1.38 (m, 1H), 0.96 (d, J=6.6 Hz, 3H), 0.92 (d, J=6.6 Hz, 3H). ESI-MS (M+H⁺)=729.

### EXAMPLE 31

### Preparation of Compound 31

Compound 31 was prepared from compound 30 in a manner similar to that described in Example 25. ¹H NMR (CDCl₃) δ 8.01-7.92 (m, 3H), 7.61-7.40 (m, 6H), 7.26-6.92 (m, 3H), 6.04 (s, 2H), 5.59-5.46 (m, 2H), 5.39 (brs, 1H), 5.14-5.19 (d, J=16.8 Hz, 1H), 4.97-4.93 (d, J=11.4 Hz, 2H), 4.58-4.54 (d, J=10.8 Hz, 1H), 4.52-4.42 (dd, J=17.1 Hz, 8.4 Hz, 1H), 4.28-4.22 (dd, J=16.8 Hz, 9.0 Hz, 1H), 4.13-4.06 (m, 1H), 3.95 (s, 3H), 2.61-2.53 (m, 2H), 2.23-2.04 (m, 1H), 2.04-1.88 (m, 1H), 1.87-1.84 (m, 1H), 1.82-1.57 (m, 8H), 1.37-1.32 (m, 1H), 1.00 (d, J=6.9 Hz, 3H), 0.98 (d, J=6.9 Hz, 3H). ESI-MS (M+H⁺)=868.

Compound I-32 was prepared as follows:

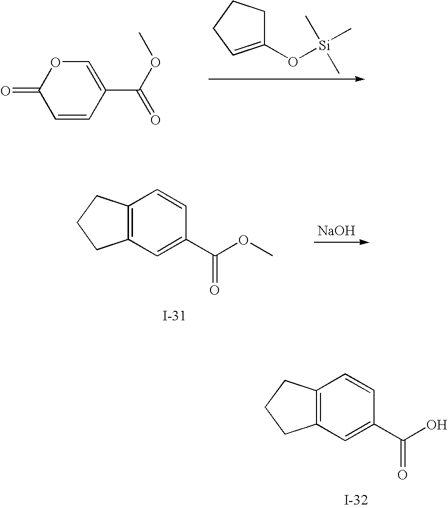

A mixture of methyl coumalate (0.15 g, 1 eq.) and 1-(trimethylsilyloxy)-cyclopentene (0.155 g, 1 eq.) was kept at 180° C. for 20 hours in a sealed vessel. The mixture was cooled, concentrated under vacuum, and purified by silica gel column chromatography to afford compound I-31 as a white solid (0.16 g, 90%). ESI-MS (M+H⁺)=177.

Compound I-31 was hydrolyzed by aq. NaOH to obtain compound I-32. ESI-MS (M+H⁺)=163.

### EXAMPLE 32

### Preparation of Compound 32

Compound 32 was prepared in a manner similar to that described for compound 24. ¹H NMR (CDCl₃) δ 8.29 (brs, 1H), 8.12-8.09 (d, J=9.6 Hz, 1H), 7.98-7.79 (m, 3H), 7.31-7.16 (m, 2H), 7.11 (s, 1H), 5.83-5.70 (m, 1H), 5.65 (brs, 1H), 5.51-4.41 (m, 1H), 5.28-5.22 (d, J=17.4 Hz, 1H), 5.11-5.08 (d, J=10.2 Hz, 1H), 4.83-4.74 (m, 2H), 4.46-4.42 (d, J=12.9 Hz, 1H), 4.21-4.09 (m, 2H), 4.03 (s, 3H), 3.01-2.77 (m, 4H), 2.39-2.08 (m, 4H), 1.79-1.44 (m, 12H), 0.97 (d, J=6.6 Hz, 3H), 0.93 (d, J=6.6 Hz, 3H). ESI-MS (M+H⁺)=725.

### EXAMPLE 33

### Preparation of Compound 33

Compound 33 was prepared from compound 32 in a manner similar to that described in Example 25. ¹H NMR (CDCl₃) δ 7.99-7.97 (d, J=7.8 Hz, 2H), 7.91 (s, 1H), 7.78-7.76 (d, J=6.9 Hz, 1H), 7.58-7.55 (m, 1H), 7.49-7.44 (m, 3H), 7.35-7.33 (d, J=7.8 Hz, 1H), 7.19 (s, 1H), 7.02-6.96 (m, 2H), 5.62-5.58 (m, 2H), 5.39 (brs, 1H), 5.14-5.08 (d, J=17.4 Hz, 1H), 4.97-4.93 (d, J=10.2 Hz, 1H), 4.57-4.48 (m, 2H), 4.28-4.22 (dd, J=10.7 Hz, 8.4 Hz, 1H), 4.21-4.09 (m, 1H), 3.94 (s, 3H), 3.03-2.94 (m, 4H), 2.61-2.42 (m, 2H), 2.16-2.10 (m, 2H), 1.86-1.41 (m, 12H), 0.99 (d, J=7.2 Hz, 3H), 0.97 (d, J=7.2 Hz, 3H). ESI-MS (M+H⁺)=865.

Compound I-34 was prepared as follows:

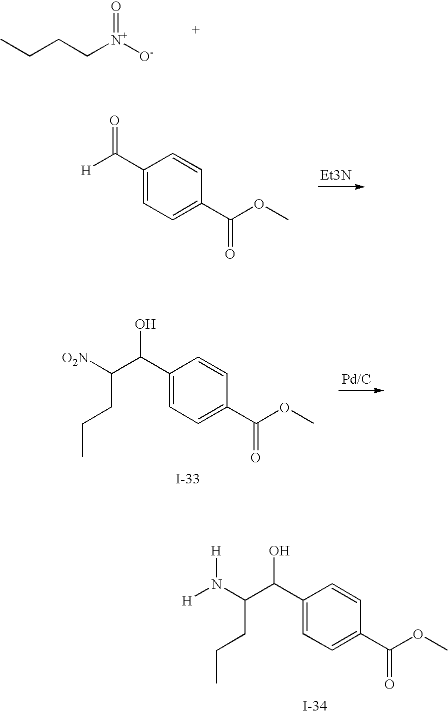

Triethyl amine (4 g, 4 eq.) was added dropwise to a stirred solution of 1-nitrobutane (1 g, 1 eq.) and methyl 4-formylbenzoate (3.18 g, 2 eq.) and MeOH (40 mL) at 0° C. over 2 hours. The solution was warmed to room temperature, stirred overnight, and concentrated to dryness to give an oil. The oil was dissolved in H₂O and acidified to PH=1 with 10% HCl, followed by extraction with EtOAc. The combined organic layer was dried over Na₂SO₄, filtered, and concentrated to dryness to give compound I-33 (2.3 g, 88%). ESI-MS (M+H⁺)=269.

To a stirred solution of compound I-33 (2 g, 1 eq.) in acetic acid (8 mL) was added 10% Pd/C (0.05 g). The resulting mixture was hydrogenated at 80 psi for 20 hours. The acetic acid was then evaporated and washed with diethyl ether and give compound I-34 (1.6 g, 90%). ESI-MS (M+H⁺)=238.

### EXAMPLE 34

### Preparation of Compound 34

Compound 34 was prepared in a manner similar to that described in Example 24 by using compound I-34 as a starting material. ¹H NMR (CDCl₃) δ 8.24-8.19 (m, 1H), 8.00-7.95 (m, 3H), 7.88-7.75 (m, 1H), 7.51-7.49 (m, 1H), 7.39-7.30 (m, 2H), 7.05-7.02 (m, 2H), 5.50-5.29 (m, 2H), 4.65-4.54 (m, 2H), 4.23-4.00 (m, 3H), 3.93 (s, 3H), 3.03 (m, 2H), 2.82 (m, 2H), 2.65-2.57 (m, 1H), 2.15-1.84 (m, 6H), 1.59-1.36 (m, 4H), 1.34 (s, 9H), 0.98-0.93 (m, 6H), 0.84-0.78 (m, 3H). ESI-MS (M+H⁺)=825.

### EXAMPLE 35

### Preparation of Compound 35

Compound 35 was prepared in a manner similar to that described in Example 24 by using compound I-36 as a starting material. ¹H NMR (CDCl₃) δ 9.35 (brs, 1H), 7.97-7.95 (m, 2H), 7.60 (s, 1H), 7.54-7.50 (d, J=8.2 Hz, 1H), 7.384-7.25 (m, 3H), 7.04-6.68 (m, 4H), 6.04 (s, 2H), 5.35-5.24 (m, 2H), 4.58-4.52 (m, 1H), 4.11-4.05 (m, 1H), 4.01-3.88 (m, 4H), 2.92 (m, 1H), 2.60-2.55 (m, 2H), 2.41 (s, 3H), 2.10-1.88 (m, 6H), 1.39 (s, 9H), 0.77 (d, J=6.0 Hz, 3H), 0.70 (d, J=6.0 Hz, 3H). ESI-MS (M+H⁺)=901.

Compound I-36 was prepared as follows:

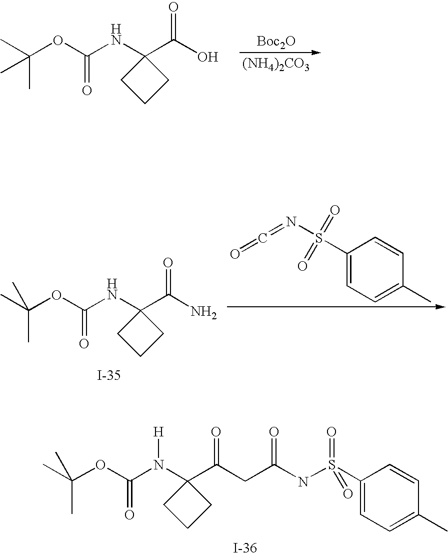

Ammonium carbonate (0.5 g, 1.26 eq.) was added to a stirred solution of 1-(tert-butoxycarbonyl)cyclobutanecarboxylic acid (1.1 g, 1 eq.), pyridine (0.25 mL), and Boc₂O (1.5 g, 1.3 eq.) in THF (8 mL). The mixture was stirred for 16 hours. After ethyl acetate was added, the solution was washed with water and 5% H₂SO₄, dried, and concentrated. The product thus obtained was triturated with ether to give I-35 (1 g, 92%). ESI-MS (M+Na⁺)=237.

A mixture of compound I-35 (0.2 g, 1 eq.) and p-toluene sulfonylisocyanate (0.2 mL, 2.3 eq) in 3 mL of toluene was heated under reflux for 1.5 hours, cooled, and concentrated under reduced pressure. The residue thus obtained was purified by silica gel column chromatography to afford compound I-36 (0.27 g, 70%). ESI-MS (M+H⁺)=412.

Prior to preparing compound 35, compound I-36 was deprotected in a manner similar to that used to prepared compound I-37.

### EXAMPLE 36

### Preparation of Compound 36

Compound 36 was prepared in a manner similar to that described for Compound 1 by using (S)-2-(cyclopentyloxycarbonyl-amino)-3-methylbutanoic acid prepared in Example 18 and I-39 as starting materials. ESI-MS (M+H⁺)=740.

Compound I-39 was prepared as follows:

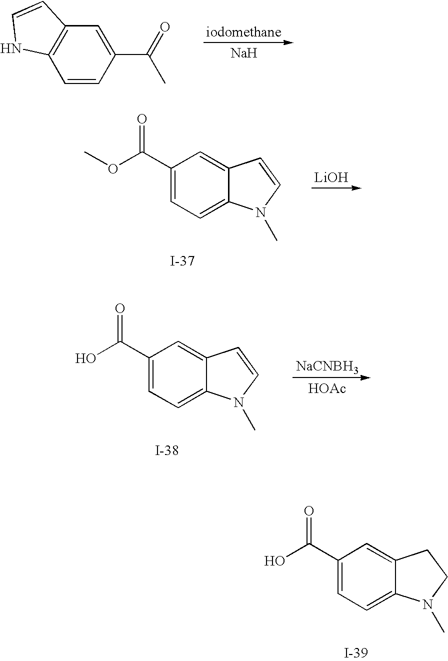

To a solution of indole-carboxylic acid (5 g, 1 eq.) in DMF (250 ml) was added dry NaH (3.75 g, 5 eq.), freshly prepared by washing 60% NaH in mineral oil with hexane. After the mixture was stirred at room temperature for 40 minutes, iodomethane (20 mL, 10 eq.) was added. After being stirred for 20 hours, the reaction mixture was poured into a 1M NaHSO₄ aqueous solution (30 mL), and extracted with ethyl acetate. The organic layer was back-extracted sequentially with water, a saturated aqueous NaHCO₃ solution, dried over MgSO₄, filtered, and concentrated to afford a crude product as an oil I-37. To a solution of the crude product I-37 in THF (80 mL) was added 80 mL of a 1 M LiOH aqueous solution. The mixture was stirred at 50° C. for 24 hours and then diluted with diethyl ether. After the mixture was then extracted twice with a saturated NaHCO₃ aqueous solution, 6 M HCl was added to the combined aqueous layers to adjust the pH to 1. The aqueous layer was then extracted with ethyl acetate. The combined ethyl acetate layer was dried over Na₂SO₄, filtered, and concentrated to give compound I-38 (4.72 g, 87%). ¹H NMR (CDCl₃) δ 8.49 (s, 1H), 8.01-7.98 (dd, J=8.4 Hz, 1.5 Hz, 1H), 7.38-7.35 (d, J=8.7 Hz, 1H), 7.14-7.13 (d, J=3.0 Hz, 1H), 6.63 (d, J=3.0 Hz, 1H), 3.84 (s, 3H). ESI-MS (M+H⁺)=176.

To a solution of compound I-38 (0.05 g, 1 eq.) in HOAc (2 mL) was added NaCNBH₃ (0.04 g, 2 eq.). The mixture was stirred at room temperature for 20 minutes. After HOAc was removed, the mixture was diluted with H₂O (5 mL) and ethyl acetate (10 mL). The mixture was then washed twice with a saturated NaHCO₃ aqueous solution, extracted with ethyl acetate (20 mL), dried over Na₂SO₄, filtered, and concentrated to give compound I-39 (0.05 g, 98%). ¹H NMR (CDCl₃) δ 7.78-7.75 (d, J=8.1 Hz, 1H), 7.63 (s, 1H), 6.35-6.32 (d, J=8.4 Hz, 1H), 3.45-3.39 (t, J=8.4 Hz, 2H), 2.91 (t, J=8.4 Hz, 2H), 2.79 (s, 3H). ESI-MS (M+H⁺)=178.

### EXAMPLE 37

### Preparation of Compound 37

Compound 37 was prepared from compound 36 in a manner similar to that described for compound 2. ESI-MS (M+H⁺)=879.

### EXAMPLE 38

### Preparation of Compound 38

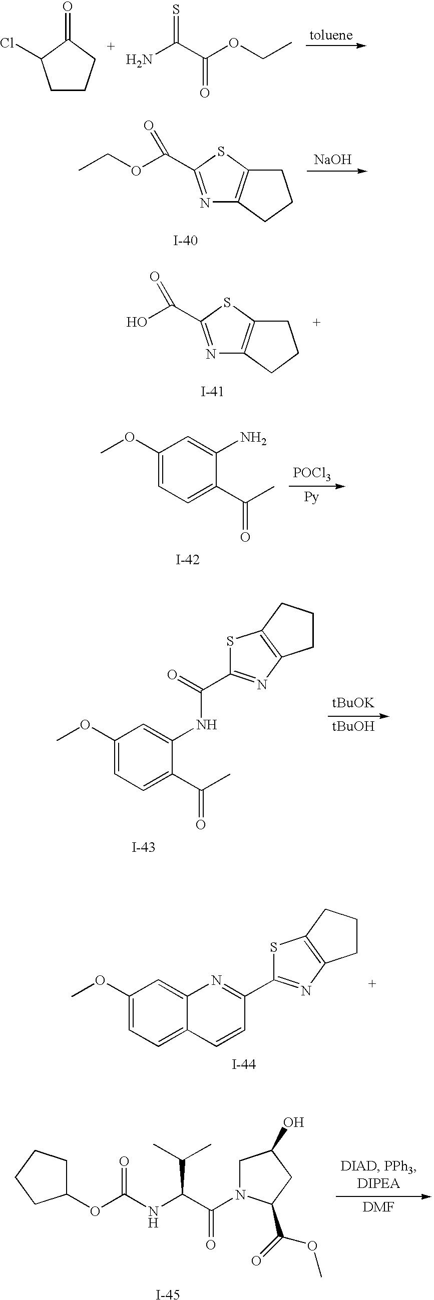

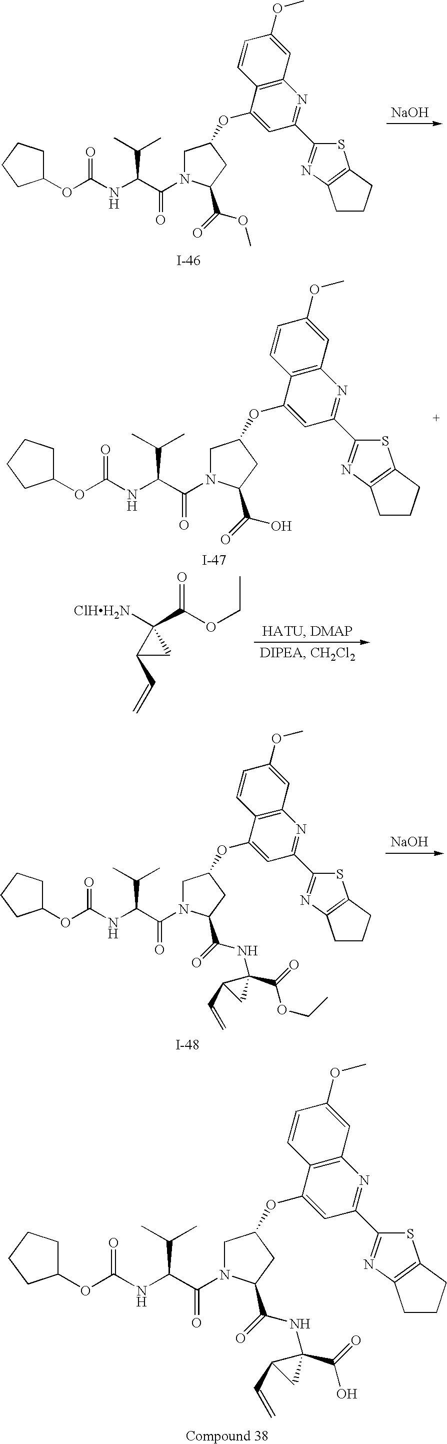

A mixture of amino-thioxo-acetic acid ethyl ester (6 g, 45 mmol) and 2-chloro-cyclopentanone (5.6 g, 47 mmol, 1.05 eq.) in toluene was heated under reflux for 4 hours. The brown solution thus obtained was cooled to room temperature, diluted with EtOAc (50 mL), washed with sat. aq. NaHCO₃ (50 mL) and brine (50 mL), dried over anhydrous MgSO₄, filtered, and concentrated under vacuum. The beige solid was purified by flash chromatography on a silica gel column (10% EtOAc in hexane) to afford compound I-40 (7.6 g, 86%) as a pale brown sticky oil. ESI-MS (M+H⁺)=198.

A solution of compound I-40 (2 g, 10 mmol) in THF/MeOH/H₂O (4:1:1, 30 mL) was treated with a 2 N NaOH aqueous solution (7.5 mL, 1.5 eq.) at room temperature for 5 hours. The mixture was concentrated to dryness under vacuum to obtain compound I-41, which was used directly in the next step without further purification. ESI-MS (M+H⁺)=170.

A solution of 4-methoxy-2-amino-acetophenone (I-42, 1.67 g, 10 mmol) and compound I-41 (10 mmol, 1 eq.) in pyridine (80 mL) was cooled to −30° C. using a cooling bath. Phosphorus oxy chloride (2.8 mL, 30 mmol, 3 eq.) was then added dropwise over a period of 15 minutes. After the reaction was stirred at −30° C. for 0.5 hours, the bath was removed and the reaction mixture was allowed to warm-up to room temperature. After the reaction mixture was stirred for 2 hours, it was poured into ice water. The pH of the mixture was adjusted to 11 with a 2 N NaOH aqueous solution and extracted with CH₂Cl₂. The organic layer was dried over anhydrous MgSO₄, filtered, and concentrated under vacuum. The crude product was purified by flash chromatography on a silica gel column (30% EtOAc in hexane) to give compound I-43 (1.1 g, 35%) as a pale beige solid. ESI-MS (M+H⁺)=317.

t-BuOK (1.0 g, 8.8 mmol, 4 eq.) was added to a suspension of compound I-43 (0.7 g, 2.2 mmol) in anhydrous t-BuOH (10 mL). The reaction mixture was heated under reflux for 2 hours, cooled to room temperature, and acidified with the addition of HCl (4N in dioxane, 3 mL). The mixture was concentrated under vacuum and the residue obtained was poured into a solution of 10% KHSO₄. After filtration, the solid was washed with ether and water, and dried under vacuum to give compound I-44 (0.4 g, 61%) as beige solid. ¹H NMR (CDCl₃-CD₃OD) δ 8.41 (d, J=8.7 Hz, 1H), 7.46-7.72 (m, 2H), 7.26-7.38 (m, 1H), 3.59 (s, 1H), 3.55 (s, 2H), 3.28 (t, J=6.6 Hz, 2H), 3.18 (t, J=6.6 Hz, 2H), 2.76-2.90 (m, 2H). ESI-MS (M+H⁺)=299.

A solution of compound I-45 (0.3 g, 1.01 mmol), compound I-44 (0.36 g, 1.01 mmol), and triphenylphosphine (0.53 g, 2.02 mmol) in DMF (10 mL) was cooled down to 0° C. Diisopropyl. azodicarboxylate (DIAD, 0.4 mL, 2.02 mmol) was added dropwise in 15 minutes. The reaction mixture was then allowed to warm up slowly to room temperature and was stirred continuously overnight. After the solvent was removed under vacuum, the mixture was diluted with CH₂Cl₂ (50 mL), washed with water (50 mL) and brine (50 mL), dried over anhydrous MgSO₄, filtered, and concentrated under vacuum. The residue was purified by flash chromatography on a silica gel column (50% EtOAc in hexane) to give compound I-46 (0.86 g, contaminated with triphenylphosphate oxide) as a yellow solid. ESI-MS (M+H⁺)=637.

A 2 N NaOH aqueous solution (10 mL) was added to a solution of compound I-46 (0.86 g, contaminated with triphenylphosphate oxide) in THF (40 mL). An additional 10 mL of MeOH was added to obtain a homogeneous solution and the resulting solution was stirred at room temperature for 4 hours. The mixture was acidified with 10% KHSO₄ to pH 3, and then extracted twice with CH₂Cl₂. The organic layer was dried over anhydrous MgSO₄, filtered, and concentrated under vacuum. The residue was purified by flash chromatography on a silica gel column (10% MeOH in CH₂Cl₂) to give compound I-47 (0.45 g, 0.7 mmol, 71% in two steps) as a yellow solid. ESI-MS (M+H⁺)=623.

A solution of acid compound I-47 (0.20 g, 0.32 mmol), HATU (0.24 g, 0.64 mmol), and DMAP (0.04 g, 0.32 mmol) in CH₂Cl₂ (10 mL) was stirred at room temperature for 0.5 hours, followed by addition of ethyl 1-amino-2-vinylcyclopropane-carboxylate (0.082 g, 0.32 mmol) and DIPEA (0.23 mL, 1.28 mmol) in CH₂Cl₂ (5 mL). After the addition was complete, the reaction mixture was stirred at room temperature for another 6 hours, diluted with CH₂Cl₂ (50 mL), washed with water (50 mL) and brine (50 mL), dried over anhydrous MgSO₄, filtered, and concentrated under vacuum. The residue was purified by flash chromatography on a silica gel column (50% EtOAc in hexane) to give compound I-48 (0.21 g, 0.27 mmol, 86%) as a yellow solid. ESI-MS (M+H⁺)=760.

A 2N NaOH (6 mL) aqueous solution was added to a solution of compound I-48 (0.21 g, 0.27 mmol) in THF (25 mL) was added. After an additional 6 mL of MeOH was then added to obtain a homogeneous solution, the resulting solution was stirred at room temperature for 4 hours. The mixture was acidified with 10% KHSO₄ to pH 3, and then extracted twice with CH₂Cl₂. The organic layer was dried over anhydrous MgSO₄, filtered, and concentrated under vacuum. The residue was purified by flash chromatography on a silica gel column (10% MeOH in CH₂Cl₂) to give compound 38 (0.16 g, 0.21 mmol, 81%) as a yellow solid. ¹H NMR (CDCl₃) δ 9.49 (brs, 1H), 7.95 (brs, 1H), 7.85 (d, J=7.5 Hz, 1H), 7.39 (s, 1H), 7.29 (s, 1H), 6.80-7.14 (m, 1H), 5.78-6.02 (m, 1H), 5.54-5.42 (m, 1H), 5.06-5.52 (m, 1H), 4.84-5.04 (m, 2H), 4.78 (s, 1H), 3.92-4.60 (m, 3H), 3.90 (s, 3H), 3.70-3.74 (m, 3H), 2.82-3.36 (m, 4H), 2.42-2.64 (m, 3H), 1.26-2.20 (m, 10H), 0.68-1.16 (m, 6H). ESI-MS (M+H⁺)=732.

Compound I-42 was prepared as follows: A solution of m-anisidine (10.9 g, 86.4 mmol) in CH₂Cl₂ (50 mL) at −78° C. was slowly treated with a solution of BCl₃ in CH₂Cl₂ (1 M, 86 mL, 86 mmol) to give a slurry, which was stirred at −50° C. for 1 hour. Acetyl chloride (11.5 g, 86 mmol) and AlCl₃ (11.5 g, 86 mmol) were added sequentially. The reaction mixture was then allowed to warm up slowly to room temperature and stirred continuously overnight. The solution was poured into ice and the pH of the mixture was adjusted to above 7 by using a 2 N NaOH aqueous solution. The mixture was then extracted with EtOAc (4×250 mL). The combined organic layer was washed with brine, dried over anhydrous MgSO₄, filtered, and concentrated under vacuum. The residue was purified by flash chromatography on a silica gel column (20% EtOAc in hexane) to give compound I-42 (6.1 g, 37 mmol, 43% yield) as a white solid: ESI-MS (M+H⁺)=166.

Compound I-45 was prepared as follows: CH₂Cl₂ (23.7 mL) was added to a mixture of cyclopentyloxycarbonyl-L-valine obtained in Example 18 (1.088 g, 4.745 mmol), HOBt (0.962 g, 7.118 mmol), and EDC (1.365 g, 7.118 mmol). 4-Methylmorpholine (2.1 mL) and H-cis-Hyp-OMe.HCl (0.862 g, 4.745 mmol) were added sequentially. After the reaction mixture was stirred at room temperature overnight, it was partitioned between H₂O (5 ml) and EtOAc (25 ml). The organic layer was separated and the aqueous layer was acidified to pH=2 with 10% KHSO_{4(aq)} and then extracted with EtOAc (3×10 ml). The organic layers were combined, dried over Na₂SO₄, and concentrated. The residue was purified by column chromatography (70% ethyl acetate in n-hexane˜2% CH₃OH in CH₂Cl₂) to give compound I-45 as a oily syrup (1.40 g, 83%). ESI-MS (M+H⁺)=357.

### EXAMPLE 39

### Preparation of Compound 39

A solution of acid compound 38 (0.07 g, 0.1 mmol), HATU (0.07 g, 0.2 mmol) and DMAP (0.01 g, 0.1 mmol) in CH₂Cl₂ (5 mL) was stirred at room temperature for 0.5 hours, followed by addition of benzenesulfonamide (0.03 g, 0.1 mmol), DIPEA (0.07 mL, 0.40 mmol) and DBU (0.02 mL, 0.1 mmol). After the addition was complete, the reaction mixture was stirred at room temperature for 6 hours, diluted with CH₂Cl₂ (50 mL), washed with water (50 mL) and brine (50 mL), dried over anhydrous MgSO₄, filtered, and concentrated under vacuum. The residue was purified by flash chromatography on a silica gel column (30% EtOAc in hexane) to give compound 39 (0.027 g, 0.03 mmol, 30%) as a yellow solid. ¹H NMR (CDCl₃) δ 7.92-7.82 (m, 3H), 7.52-7.62 (m, 1H), 7.42-7.52 (m, 3H), 7.32 (d, J=2.1 Hz, 1H), 7.15 (s, 1H), 6.99 (dd, J=9.0 Hz, 2.4 Hz, 1H), 5.64 (d, J=9.3 Hz, 1H), 5.55 (dd, J=16.5 Hz, 8.1 Hz, 1H), 5.43 (brs, 1H), 5.13 (s, 0.5H), 5.07 (s, 0.5H), 4.86-4.98 (m, 3H), 4.52 (d, J=11.7 Hz, 1H), 4.44 (dd, J=9.9 Hz, 6.9 Hz, 1H), 4.21 (t, J=8.4 Hz, 1H), 4.057 (dd, J=11.7 Hz, 3.6 Hz, 1H), 3.93 (s, 3H), 2.86-3.04 (m, 4H), 2.40-2.66 (m, 4H), 1.26-2.24 (m, 12H), 0.98 (d, J=6.6 Hz, 3H), 0.94 (d, J=6.6 Hz, 3H). ESI-MS (M+H⁺)=871.

### EXAMPLE 40

### Preparation of Compound 40

Compound 40 was prepared in a manner similar to that described in Example 38. ¹H NMR (CDCl₃) δ 9.50 (brs, 1H), 7.86-8.06 (m, 1H), 7.85 (d, J=8.1 Hz, 1H), 7.42 (s, 1H), 7.30 (s, 1H), 6.91-7.14 (brs, 1H), 6.80-6.90 (m, 1H), 5.78-6.06 (m, 1H), 5.322 (brs, 1H), 5.08 (d, J=16.8 Hz, 1H), 4.40-5.52 (m, 2H), 3.74-4.36 (m, 6H), 3.43 (brs, 4H), 2.85 (s, 4H), 2.46-2.64 (m, 1H), 1.24-2.21 (m, 13H), 0.76-1.16 (m, 6H). ESI-MS (M+H⁺)=746.

### EXAMPLE 41

### Preparation of Compound 41

Compound 41 was prepared from compound 40 in a manner similar to that described in example 39. ¹H NMR (CDCl₃) δ 7.95 (d, J=8.1 Hz, 2H), 7.56 (t, J=7.2 Hz, 1H), 7.40-7.50 (m, 3H), 7.32 (d, J=2.1 Hz, 1H), 7.14 (brs, 1H), 6.99 (dd, J=9.3 Hz, 2.1 Hz, 1H), 5.48-5.66 (m, 2H), 5.45 (brs, 1H), 5.11 (d, J=17.1 Hz, 1H), 4.86-4.88 (m, 2H), 4.40-4.56 (m, 2H), 4.21 (t, J=8.4 Hz, 1H), 4.07 (dd, J=11.7 Hz, 3.6 Hz, 1H), 3.93 (s, 3H), 2.85 (brs, 4H), 2.54-2.66 (m, 1H), 2.40-2.52 (m, 1H), 1.22-2.24 (m, 12H), 0.98 (d, J=6.3 Hz, 3H), 0.94 (d, J=6.3 Hz, 3H). ESI-MS (M+H⁺)=885.

### EXAMPLE 42

### Preparation of Compound 42

Compound 42 was prepared in a manner similar to that described in Example 38. ¹H NMR (CDCl₃) δ 9.83 (brs, 1H), 7.92-8.08 (m, 1H), 7.83 (d, J=8.7 Hz, 1H), 7.29-7.48 (m, 2H), 6.87-7.20 (m, 2H), 5.72-5.94 (m, 1H), 5.34 (brs, 1H), 5.13 (d, J=17.4 Hz, 1H), 4.94 (d, J=9.3 Hz, 1H), 4.50-4.64 (m, 1H), 3.54-4.28 (m, 7H), 2.86 (s, 4H), 2.54-2.70 (m, 1H), 1.50-2.46 (m, 7H), 1.23 (s, 6H), 0.74-1.12 (m, 6H). ESI-MS (M+H⁺)=734.

### EXAMPLE 43

### Preparation of Compound 43

Compound 43 was prepared from compound 42 in a manner similar to that described in Example 39. ¹H NMR (CDCl₃) δ 7.86-8.02 (m, 2H), 7.26-7.54 (m, 7H), 7.01 (d, J=8.6 Hz, 1H), 5.30-5.72 (m, 2H), 5.08 (d, J=17.4 Hz, 1H), 4.92 (d, J=10.2 Hz, 1H), 4.40-4.62 (m, 1H), 3.98-4.22 (m, 2H), 3.93 (s, 3H), 2.93 (s, 1H), 2.85 (s, 4H), 250-2.68 (m, 1H), 2.32-2.50 (m, 1H), 1.64-2.18 (m, 6H), 1.33 (s, 6H), 0.84-1.16 (m, 6H). ESI-MS (M+H⁺)=873.

### EXAMPLE 44

### Preparation of Compound 44

Compound 44 was prepared in a manner similar to that described in Example 38. ¹H NMR (CDCl₃) δ 7.84 (brs, 1H), 6.82-7.40 (m, 5H), 6.57 (brs, 1H), 5.81 (brs, 2H), 4.72-5.38 (m, 4H), 3.70-3.4.20 (m, 5H), 3.10-3.60 (m, 2H), 2.40-2.90 (m, 5H), 1.60-2.16 (m, 6H). ESI-MS (M+H⁺)=683.

### EXAMPLE 45

### Preparation of Compound 45

Compound 45 was prepared from compound 44 in a manner similar to that described in Example 39. ¹H NMR (CDCl₃) δ 7.84 (d, J=9.6 Hz, 1H), 7.68 (t, J=6.0 Hz, 1H), 7.59 (brs, 1H), 7.42 (brs, 1H), 7.30 (d, J=2.4 Hz, 2H), 7.02-7.14 (m, 3H), 6.74 (d, J=8.7 Hz, 1H), 5.96 (s, 2H), 5.91 (dd, J=17.1 Hz, 7.8 Hz, 1H), 5.33 (brs, 1H), 5.21 (dd, J=17.1 Hz, 1.5 Hz, 1H), 5.03 (dd, J=10.2 Hz, 1.8 Hz, 1H), 4.92 (t, J=9.0 Hz, 1H), 4.14 (dd, J=12.3 Hz, 3.3 Hz, 1H), 3.93 (s, 3H), 3.32-3.45 (m, 1H), 3.18-3.28 (m, 3H), 3.00-3.16 (m, 1H), 2.77-2.89 (m, 4H), 2.56-2.68 (m, 1H), 2.37-2.45 (m, 2H), 1.48-2.08 (m, 7H). ESI-MS (M+H⁺)=822.

### EXAMPLE 46

### Preparation of Compound 46

Compound 46 was prepared in a manner similar to that described in Example 39. ¹H NMR (CDCl₃) δ 7.80-8.08 (m, 3H), 7.20-7.54 (m, 6H), 6.92-7.04 (m, 1H), 6.76-6.84 (m, 1H), 5.72-5.88 (m, 1H), 5.22-5.42 (m, 2H), 5.04-5.16 (m, 1H), 4.78-4.88 (m, 1H), 4.64-4.76 (m, 1H), 4.50-4.60 (m, 1H), 4.23-4.38 (m, 1H), 3.84-4.22 (m, 5H), 3.74 (t, J=9.3 Hz, 1H), 3.56-5.70 (m, 1H), 3.16-3.32 (m, 1H), 2.62-2.92 (m, 5H), 2.42-2.58 (m, 1H), 1.26-2.31 (m, 14H), 0.76-1.00 (m, 6H). ESI-MS (M+H⁺)=873.

### EXAMPLE 47

### Preparation of Compound 47

Compound 47 was prepared in a manner similar to that described in compound 1 using compound I-54 as a starting material. ¹H NMR (CDCl₃) δ 7.78-7.92 (m, 3H), 7.42-7.52 (m, 2H), 7.35 (d, J=2.1 Hz, 1H), 6.98 (dd, J=9.0 Hz, 2.1 Hz, 1H), 6.88 (s, 1H), 6.79 (d, J=7.8 Hz, 1H), 5.92 (s, 2H), 5.64-5.82 (m, 1H), 2.07 (dd, J=17.4 Hz, 8.7 Hz, 1H), 5.00 (d, J=10.5 Hz, 1H), 4.72 (t, J=7.5 Hz, 1H), 4.48 (d, J=11.4 Hz, 1H), 4.22 (d, J=10.8 Hz, 1H), 3.90 (s, 3H), 3.74-3.82 (m, 1H), 3.12-3.32 (m, 2H), 3.00-3.12 (m, 1H), 2.75 (brs, 5H), 2.34-2.50 (m, 1H), 2.14-2.28 (m, 1H), 2.07 (dd, J=17.4 Hz, 8.7 Hz, 1H), 1.18-1.82 (m, 8H), 0.82-0.94 (m, 3H), 0.77 (d, J=6.6 Hz, 3H). ESI-MS (M+H⁺)=754.

Compound I-54 was prepared as follows:

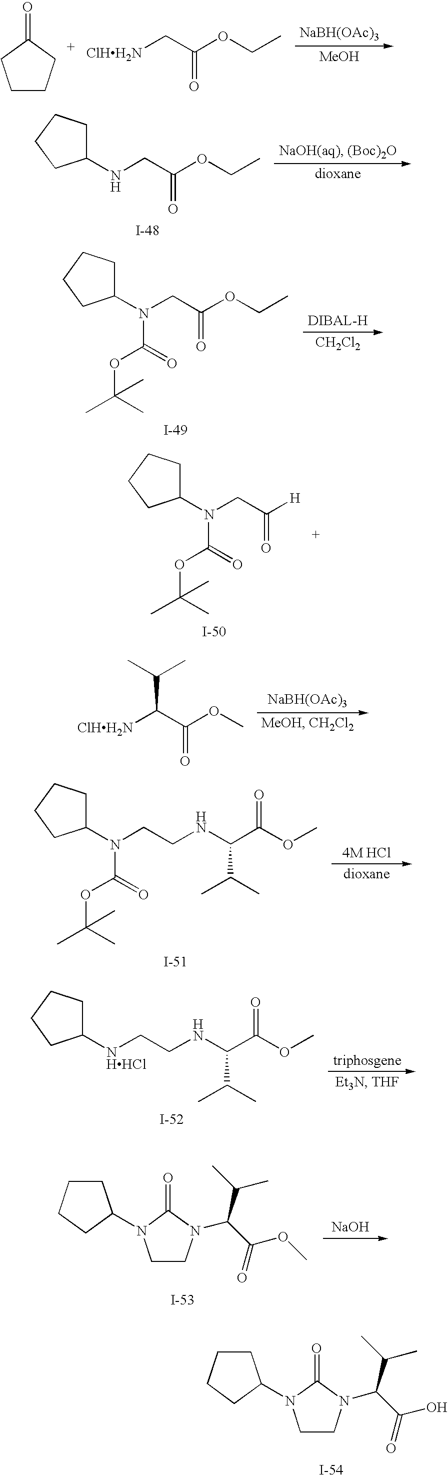

Solid sodium triacetoxyborohydride (16.7 g, 78.8 mmol) was added to cyclopentanone (10 g, 72.0 mmol) and ethyl glycinate hydrochloride (7.0 mL, 78.8 mmol) in 100 mL MeOH at room temperature. The reaction was stirred at room temperature overnight. After an HCl aqueous solution (1 N, 50 mL) was added, the mixture was stirred for 1 hour and was rotary evaporated. The residue was dissolved in 70 mL of 1 N NaOH and extracted with CH₂Cl₂ (3×100 mL). The combined organic layers were washed with brine, dried over anhydrous MgSO₄, filtered, and concentrated under vacuum to give compound I-48, which was used in the next step without further purification. ESI-MS (M+H⁺)=172.

To a solution of compound I-48 in 100 mL dioxane was added a 2N NaOH aqueous solution to give a clean solution. (Boc)₂O (15 mL, 70 mmol) was then added and the mixture was stirred at room temperature for 4 hours. The mixture was then extracted with CH₂Cl₂ (2×100 mL) and water (100 mL). The combined organic layer was washed with brine, dried over anhydrous MgSO₄, filtered, and concentrated under vacuum. The residue was purified by flash chromatography on a silica gel column (10% EtOAc in hexane) to give compound I-49 (11.5 g, 42 mmol, 59% yield in two steps) as an oil. ESI-MS (M+H⁺)=272.

To a solution of compound I-49 (5 g, 18.4 mmol) in 50 mL CH₂Cl₂ at −78° C. was added DIBAL (1.0 M in hexane, 28 mL, 27.6 mmol) dropwise over 20 minutes. The reaction was stirred at −75° C. for 18 hours and quenched with a NH₄Cl aqueous solution (3 mL) and an 1 N HCl aqueous solution (8 mL). After the mixture was stirred for 1 hour, 1 N NaOH (50 mL) was added. The mixture was then extracted with CH₂Cl₂ (2×50 mL). The organic layer was dried over anhydrous MgSO₄, filtered, and concentrated under vacuum. The residue was purified by flash chromatography on a silica gel column (20% EtOAc in hexane) to give compound I-50 (3.2 g, 14 mmol, 76%) as an oil. ESI-MS (M+H⁺)=228.

To a solution of the compound I-50 (3.7 g, 16.3 mmol) in CH₂Cl₂/MeOH (1:2, 60 mL) at 0° C. were added L-valine methyl ester hydrochloride (2.7 g, 16.3 mmol) and NaB(OAc)₃H (3.8 g, 17.9 mmol). The mixture was allowed to warm up to room temperature and stirred for 6 hours. Aqueous solutions of NH₄Cl (4 mL) and 1 N HCl (10 mL) were added. The mixture was stirred for 1 hour and then extracted with CH₂Cl₂ (2×100 mL) and 1 N NaOH (100 mL). The organic layer was dried over anhydrous MgSO₄, filtered, and concentrated under vacuum. The residue was purified by flash chromatography on a silica gel column (30% EtOAc in hexane) to give compound I-51 (3 g, 8.7 mmol, 53%) as an oil. ESI-MS (M+H⁺)=343.

Compound I-51 (1 g, 2.9 mmol) was treated with 4 M HCl in dioxane (6 mL) at room temperature for 2 hours. The solvent was then evaporated and the residue was re-dissolved twice in CH₂Cl₂/MeOH (1:1 ratio) to removed excess HCl. The solid was dried under high vacuum to give an amine hydrochloride salt I-52, which was used in the next step without further purification. ESI-MS (M+H⁺)=243.

To a solution of compound I-52 and triethylamine (1.7 mL, 1.2 mmol) in THF (30 mL) cooled to 0° C. by a ice-water bath was added triphosgene (0.3 g, 1 mmol). After the ice-water bath was removed, the mixture was stirred for 24 hours. Isolation of the product involved addition of 1N HCl (10 mL). The aqueous phase was separated and washed with CH₂Cl₂ (100 mL). The combined organic layer was dried over anhydrous MgSO₄, filtered, and concentrated under vacuum. The crude product was purified by flash chromatography on a silica gel column (30% EtOAc in hexane) to give compound I-53 (0.35 g, 1.3 mmol, 45% yield in two steps) as an oil. ESI-MS (M+H⁺)=269.

To a solution of compound I-53 (0.6 g, 2.2 mmol) in THF (30 mL) was added 2N NaOH (6 mL). After an additional 6 mL of MeOH was added to obtain a homogeneous solution, the resulting solution was stirred at room temperature for 4 hours. The mixture solution was acidified with 10% KHSO₄ to pH 3, and then extracted twice with CH₂Cl₂. The organic layer was dried over anhydrous MgSO₄, filtered, and concentrated under vacuum. The residue was purified by flash chromatography on a silica gel column (10% MeOH in CH₂Cl₂) to give compound I-54 (0.55 g, 2.1 mmol, 98%) as a white solid. ESI-MS (M+H⁺)=255.

### EXAMPLE 48

### Preparation of Compound 48

Compound 48 was prepared from compound 47 in a manner similar to that described in Example 2. ¹H NMR (CDCl₃) δ 7.91-8.00 (m, 3H), 7.42-7.61 (m, 6H), 7.38 (d, J=2.1 Hz, 1H), 7.12 (brs, 1H), 7.01 (dd, J=9.0 Hz, 2.7 Hz, 1H), 6.86-6.93 (m, 2H), 6.00 (s, 2H), 5.46-5.61 (m, 1H), 5.32 (brs, 1H), 5.08 (d, J=16.2 Hz, 1H), 4.91 (d, J=11.4 Hz, 1H), 4.77 (d, J=11.4 Hz, 1H), 4.37 (dd, J=9.3 Hz, 6.9 Hz, 1H), 4.25 (d, J=11.1 Hz, 1H), 3.97-4.10 (m, 2H), 3.92 (s, 1H), 3.71 (dd, J=17.1 Hz, 8.4 Hz, 1H), 3.30-3.41 (m, 1H), 3.10-3.26 (m, 2H), 2.28-2.62 (m, 3H), 0.97 (dd, J=17.7 Hz, 9.0 Hz, 2H), 1.80 (dd, J=8.1 Hz, 5.7 Hz, 1H), 1.24-1.74 (m, 8H), 0.97 (d, J=6.6 Hz, 3H), 0.94 (d, J=6.6 Hz, 3H). ESI-MS (M+H⁺)=893.

### EXAMPLE 49

### Preparation of Compound 49

Compound 49 was prepared in a manner similar to that described in Example 38 using (2S,4S)-methyl 1-((S)-2-(3-cyclopentyl-2-oxoimidazolidin-1-yl)-3-methylbutanoyl)-4-hydroxypyrrolidine-2-carboxylate as a starting material. ¹H NMR (CDCl₃) δ 7.95 (d, J=9 Hz, 1H), 7.42 (s, 1H), 7.31 (d, J=2.4 Hz, 1H), 7.05-7.13 (m, 1H), 6.51 (s, 1H), 5.82-5.98 (m, 1H), 5.42-5.55 (m, 1H), 5.34 (dd, J=15.9 Hz, 1.2 Hz, 1H), 5.14 (dd, J=11.4 Hz, 1.2 Hz, 1H), 4.65 (d, J=7.2 Hz, 1H), 4.10-4.40 (m, 3H), 3.93 (s, 3H), 3.54-3.66 (m, 1H), 3.14-3.38 (m, 3H), 3.04 (t, J=7.2 Hz, 2H), 2.93 (t, J=6.3 Hz, 2H), 2.42-2.56 (m, 2H), 2.30-2.40 (m, 4H), 1.90-2.08 (m, 1H), 1.34-1.88 (m, 9H), 1.06 (d, J=6.3 Hz, 1H), 0.91 (d, J=6.6 Hz, 1H). ESI-MS (M+H⁺)=757.

(2S,4S)-methyl 1-((S)-2-(3-cyclopentyl-2-oxoimidazolidin-1-yl)-3-methylbutanoyl)-4-hydroxypyrrolidine-2-carboxylate was prepared as follows: A solution of compound I-54 (0.50 g, 2.0 mmol), HATU (1.5 g, 4.0 mmol) and DMAP (0.24 g, 2.0 mmol) in CH₂Cl₂ (30 mL) was stirred at room temperature for 0.5 hours, then followed by addition of (2S,4S)-methyl 4-hydroxypyrrolidine-2-carboxylate hydrochloride (0.36 g, 2.0 mmol) and DIPEA (1.4 mL, 8.0 mmol) in CH₂Cl₂ (10 mL). After the addition was complete, the reaction mixture was stirred at room temperature for 18 hours, diluted with CH₂Cl₂ (50 mL), washed with water (50 mL) and brine (50 mL), dried over anhydrous MgSO₄, filtered, and concentrated under vacuum. The residue was purified by flash chromatography on a silica gel column (3% MeOH in CH₂Cl₂) to give compound I-55 (0.67 g, 1.7 mmol, 88%) as a white solid. ESI-MS (M+H⁺)=382.

### EXAMPLE 50

### Preparation of Compound 50

Compound 50 was prepared from compound 49 in a manner similar to that described in Example 39. ESI-MS (M+H⁺)=896.

### EXAMPLE 51

### Preparation of Compound 51

Compound 51 was prepared in a manner similar to that described in Example 47. ¹H NMR (CDCl₃) δ 7.74-7.90 (m, 2H), 7.36-7.54 (m, 3H), 6.98 (dd, J=9.0 Hz, 1.8 Hz, 1H), 6.91 (s, 1H), 6.79 (d, J=7.8 Hz, 1H), 5.92 (s, 2H), 5.66-5.84 (m, 1H), 5.32 (brs, 1H), 5.22 (d, J=17.1 Hz, 1H), 5.04 (d, J=10.8 Hz, 1H), 4.71 (t, J=7.8 Hz, 1H), 4.46 (d, J=11.4 Hz, 1H), 4.23 (d, J=10.8 Hz, 1H), 3.91 (s, 1H), 3.08-3.34 (m, 3H), 2.72-2.98 (m, 2H), 2.35-2.49 (m, 1H), 2.16-2.32 (m, 1H), 2.09 (dd, J=18.6 Hz, 9.9 Hz, 1H), 1.80 (t, J=6.0 Hz, 1H), 1.38-1.48 (m, 1H), 1.28 (s, 1H), 1.22 (s, 1H), 1.03 (s, 7H), 0.89 (d, J=6.0 Hz, 3H), 0.79 (d, J=6.9 Hz, 3H). ESI-MS (M+H⁺)=742.

### EXAMPLE 52

### Preparation of Compound 52

Compound 52 was prepared from compound 51 in a manner similar to that described in compound 48. ESI-MS (M+H⁺)=881.

### EXAMPLE 53

### Preparation of Compound 53

Compound 53 was prepared in a manner similar to that described in Example 49. ESI-MS (M+H⁺)=745.

### EXAMPLE 54

### Preparation of Compound 54

Compound 54 was prepared from compound 53 in a manner similar to that described in compound 50. ESI-MS (M+H⁺)=884.

### EXAMPLE 55

### Preparation of Compound 55

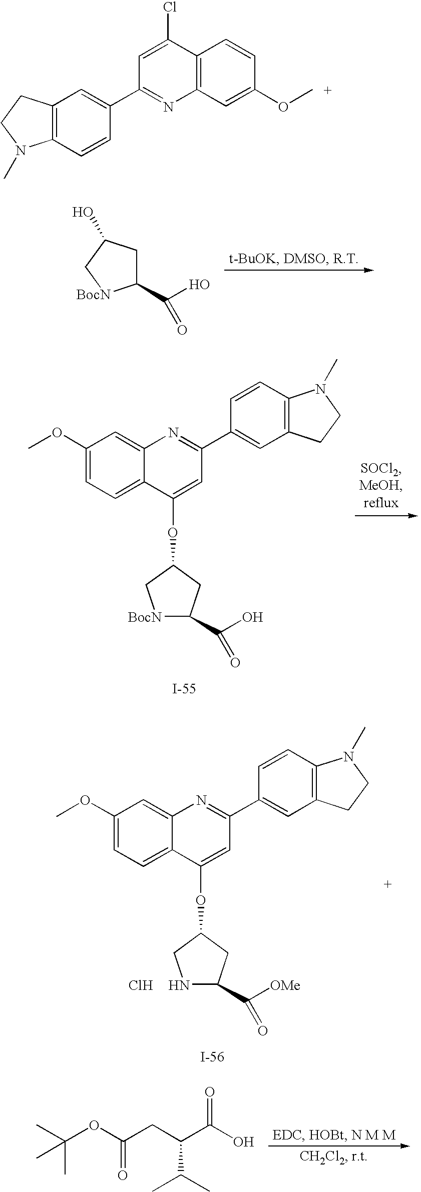

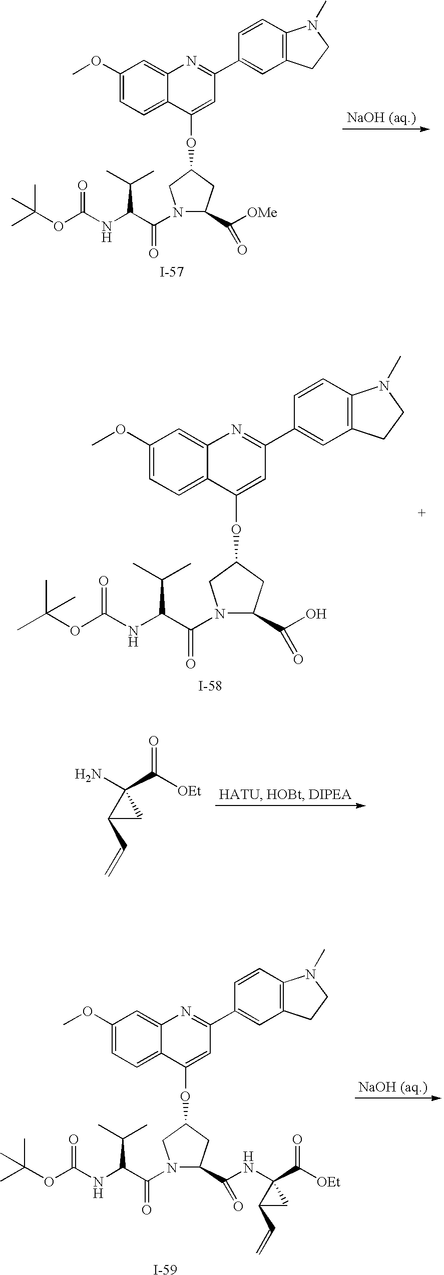

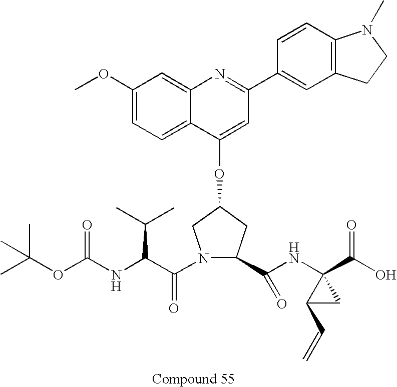

To a suspension of N-Boc-(2S,4R)-hydroxyproline (0.325 g) in dry DMSO (2 mL) was added t-BuOK (0.394 g) at 0° C. After the mixture was stirred for 1.5 hours, 4-chloro-7-methoxy-2-(1-methyl-2,3-dihydro-1H-indol-5-yl)-quinoline (0.5 g) was added in three portions over 1 hour. The mixture was stirred for one day and was poured into cold water (10 mL) and washed with Et₂O (4×10 mL). The aqueous solution was acidified to pH 4.6, filtered to obtain a white solid, and dried in vacuo to give compound I-55 (0.620 g, 85%). ESI-MS (M+H⁺)=619.

MeOH (8 mL) was cooled in an ice-NaCl bath and SOCl₂ (1 mL) was added dropwise. After compound I-55 (0.799 g) was added to the resulting solution, the mixture was refluxed for one hour. MeOH was then evaporated to give compound I-56, which was used in the next without further purification (0.722 g). ESI-MS (M+H⁺)=533.

Boc-L-Val-OH (0.401 mg) was mixed with EDC (0.53 g), HOBt (0.208 g) and NMM (0.622 mL) in CH₂Cl₂ (7 mL) and the mixture was stirred for 20 minutes. After compound I-56 (0.722 g) was added, the mixture was stirred at room temperature for 2 hours. After brine (25 mL) was added, the mixture was extracted with EtOAc (3×15 mL). The organic layer was combined, dried with MgSO₄, and concentrated. The residue was purified by flash column chromatography (3% MeOH in CH₂Cl₂) to give compound I-57 as a white solid. ESI-MS (M+H⁺)=633.

NaOH (0.335 mL, 30%) was added to a solution of compound I-57 (0.302 g) in a mixture of THF (1 mL), MeOH (8 mL) and water (1 mL). The mixture was stirred at room temperature for several hours. After the solvents were then removed under reduced pressure, the crude product was redissolved in EtOAc and diluted with brine. The pH of the aqueous layer was adjusted to 6 with HCl (1 N) and the aqueous phase was extracted with EtOAc (3×). The combined organic phase was washed with water and brine, dried over MgSO₄, and concentrated under reduced pressure to afford compound I-58 as a yellow solid (0.253 g). ESI-MS (M+H⁺)=619.

(2R,3S)-3-vinyl-2-aminocyclopropyl carboxylic acid ethyl ester hydrochloride (0.046 g) and compound I-58 (0.150 g) in CH₂Cl₂ (5 mL) were added to a mixture of HATU (0.187 g), HOBt (0.039 g), and DIPEA (0.126 mL). The mixture was stirred at room temperature under N₂ for several hours. It was then concentrated under reduced pressure, diluted with EtOAc, washed with HCl, NaHCO₃ and brine solutions. The organic phase was then dried with MgSO₄ and concentrated under reduced pressure to afford compound I-59 (0.080 g). ESI-MS (M+H⁺)=756.

NaOH (0.3 mL, 30%) was added to a solution of compound I-59 (0.08 g) in a mixture of THF (1 mL), MeOH (8 mL), and water (1 mL). The reaction mixture was stirred at room temperature for several hours. After the solvents were removed under reduced pressure, the crude product was redissolved in EtOAc and diluted with brine. The pH of the aqueous layer was adjusted to 6 with HCl (1 N). The aqueous phase was extracted with EtOAc (3×). The combined organic phase was washed with water and brine, dried over MgSO₄, and concentrated under reduced pressure to afford Compound 55 as a yellow solid (0.693 g, 90%). ESI-MS (M+H⁺)=728.

### EXAMPLE 56

### Preparation of Compound 56

A solution of compound 55 (0.110 g), HATU (0.115 g), and DIEA (0.078 mL) in dry DMF (5 mL) was stirred for 1 hour before addition of a solution of benzenesulfonamide (0.048 g), DMAP (0.019 g), and DBU (0.046 mL) in dry DMF (1.5 mL). The mixture was stirred overnight, diluted with EtOAc (60 mL), and washed with an NaOAc aqueous buffer (pH 4, 2×15 mL), a 5% NaHCO₃ aqueous solution (15 mL) and brine (20 mL). The organic layer was dried over anhydrous MgSO₄, filtered, and concentrated. The residue was purified by preparative HPLC to give Compound 56 as a white solid (0.04 g, 31%). ESI-MS (M+H⁺)=868.

### EXAMPLE 57

### Preparation of Compound 57

Compound 57 was prepared (0.10 g, 77%) in a manner similar to that described in Example 55. ESI-MS (M+H⁺)=782.

### EXAMPLE 58

### Preparation of Compound 58

Compound 58 was prepared (0.013 g, 35%) from compound 57 in a manner similar to that described in Example 56. ESI-MS (M+H⁺)=894.

### EXAMPLE 59

### Preparation of Compound 59

Compound 59 was prepared (50 mg, 70%) in a manner similar to that described of the preparation of compound I-11 described in Example 1 by using tert-butyl 1-(thiophen-2-ylcarbamoylcarbamoyl)cyclobutylcarbamate as a starting material. ESI-MS (M+H⁺)=829.

tert-Butyl 1-(thiophen-2-ylcarbamoylcarbamoyl)cyclobutylcarbamate was prepared (0.26 g, 60%) in a manner similar to that of the preparation of compound I-16 described in Example 14. ESI-MS (M+H⁺)=339.

### EXAMPLE 60

### Preparation of Compound 60

Compound 60 was prepared (60 mg, 70%) in a manner similar to that of the preparation of compound I-11 described in Example 1 by using tert-butyl 1-(phenylsulfonylcarbamoylcarbamoyl)cyclobutylcarbamate as a staring material. ESI-MS (M+H⁺)=887.

tert-Butyl 1-(phenylsulfonylcarbamoylcarbamoyl)cyclobutylcarbamate was prepared (0.17 g, 30%) in a manner similar to that of the preparation of compound I-16 described in Example 14. ESI-MS (M+H⁺)=398.

### EXAMPLE 61

### Preparation of Compound 61

Compound 61 was prepared (0.1 g, 60%) in a manner similar to that of the preparation of compound I-11 described in Example 1 by using ethyl 2-(1-(tert-butoxycarbonylamino)cyclobutanecarboxamido)-2-hydroxyacetate as a staring material. ESI-MS (M+H⁺)=806.

Ethyl 2-(1-(tert-butoxycarbonylamino)cyclobutanecarboxamido)-2-hydroxyacetate was prepared as follows: Ethyl glyoxalate (0.36 g, 3.5 mmol) was added to a solution of tert-butyl 1-carbamoylcyclobutylcarbamate (0.3 g, 1.4 mmol) in acetone (10 mL) at room temperature. The solution was heated under reflux overnight and then concentrated under vacuum. The residue was purified by silica gel column chromatography to give the desired compound (0.2 g, 46%). ESI-MS (M+H⁺)=317.

### EXAMPLE 62

### Preparation of Compound 62

To a solution of compound 61 (0.1 g, 0.12 mmol) in CH₂Cl₂ (10 mL) was added Dess-Martine periodnane (80 mg, 0.018 mmol) at room temperature. The solution was stirred for 1 hour. The reaction was then quenched by water and concentrated under vacuum. The residue was purified by silica gel column chromatography to give compound 62 (14 mg, 15%). ESI-MS (M+H⁺)=806.

### EXAMPLE 63

### Preparation of Compound 63

Compound 63 was prepared in a manner similar to that described in Example 9. LC/MS (M+H⁺)=832.30.

### EXAMPLE 64

### Preparation of Compound 64

Compound 64 was prepared in a manner similar to that described in Example 9. LC/MS (M+H⁺)=832.30; ¹H NMR δ 10.15 (brs, 1H), 7.97 (d, J=9.0 Hz, 1H), 7.60 (s, 1H), 7.53 (d, J=8.1 Hz, 1H), 7.38 (s, 1H), 7.37-7.29 (m, 1H), 7.04 (d, J=9.3 Hz, 1H), 6.93 (d, J=8.1 Hz, 1H), 6.89 (s, 1H), 6.03 (s, 2H), 5.86-5.73 (m, 1H), 5.68 (d, J=7.2 Hz, 1H), 5.35 (brs, 1H), 5.23 (d, J=16.8 Hz, 1H), 5.11 (d, J=10.5 Hz, 1H), 4.89 (brs, 1H), 4.57-4.55 (m, 2H), 4.20 (dd, J=8.7 Hz, J=8.7 Hz, 1H), 4.18-4.05 (m, 2H), 3.94 (s, 3H), 2.94-2.82 (m, 1H), 2.64-2.56 (m, 1H), 2.53-2.41 (m, 1H), 2.18-1.92 (m, 3H), 1.86-1.38 (m, 8H), 1.34-1.16 (m, 4H), 1.06-0.88 (m, 6H).

### EXAMPLE 65

### Preparation of Compound 65

Compound 65 was prepared in a manner similar to that described in Example 9. LC/MS (M+H⁺)=882.30.

### EXAMPLE 66

### Preparation of Compound 66

Compound 66 was prepared in a manner similar to that described in Example 9. LC/MS (M+H⁺)=848.30.

### EXAMPLE 67

### Preparation of Compound 67

Compound 67 was prepared in a manner similar to that described in Example 9. LC/MS (M+H⁺)=847.00.

### EXAMPLE 68

### Preparation of Compound 68

Compound 68 was prepared in a manner similar to that described in Example 9. LC/MS (M+H⁺)=835.00.

### EXAMPLE 69

### Preparation of Compound 69

Compound 69 was prepared in a manner similar to that described in Example 37. LC/MS (M+H⁺)=834.90; ¹H NMR δ 10.17 (s, 1H), 7.98 (d, J=9.3 Hz, 1H), 7.47 (s, 1H), 7.38 (s, 1H), 7.06 (d, J=7.5 Hz, 2H), 5.81 (m, 1H), 5.54 (d, J=8.7 Hz, 1H), 5.46 (s, 1H), 5.23 (d, J=17.1 Hz, 1H), 5.12 (d, J=10.2 Hz, 1H), 4.90 (s, 1H), 4.52 (d, J=11.7 Hz, 1H), 4.44 (t, J=8.1 Hz, 1H), 4.21 (t, J=8.4 Hz, 1H), 4.15-4.05 (m, 2H), 3.96 (s, 3H), 3.03-2.92 (m, 4H), 2.67-2.49 (m, 4H), 2.14-1.96 (m, 4H), 1.72-1.41 (m, 8H), 1.25 (t, J=7.2 Hz, 4H), 0.99 (d, J=6.6 Hz, 3H), 0.94 (d, J=6.9 Hz, 3H).

### EXAMPLE 70

### Preparation of Compound 70

Compound 70 was prepared in a manner similar to that described in Example 37. LC/MS (M+H⁺)=837.30.

### EXAMPLE 71

### Preparation of Compound 71

Compound 71 was prepared in a manner similar to that described in Example 1. LC/MS (M+H⁺)=843.40.

### EXAMPLE 72

### Preparation of Compound 72

Compound 72 was prepared in a manner similar to that described in Example 1. LC/MS (M+H⁺)=843.50; ¹H NMR δ 8.27 (d, J=7.8 Hz, 1H), 8.00 (d, J=9.0 Hz, 1H), 7.79 (s, 1H), 7.51 (s, 1H), 7.50 (d, J=7.8 Hz, 1H), 7.40 (s, 1H), 7.06 (dd, J=9.0 Hz, J=2.4 Hz, 1H), 5.85-5.77 (m, 2H), 5.49 (s, 1H), 5.21 (d, J=17.4 Hz, 1H), 5.08 (d, J=10.5 Hz, 1H), 4.89 (brs, 1H), 4.53-4.48 (m, 2H), 4.21 (dd, J=9.0 Hz, J=9.0 Hz, 1H), 3.94 (s, 3H), 3.04-2.99 (m, 2H), 2.85-2.75 (m, 3H), 2.66-2.60 (m, 1H), 2.48-2.24 (m, 1H), 2.15-1.1.81 (m, 9H), 1.80-1.26 (m, 9H), 0.99-0.88 (m, 9H).

### EXAMPLE 73

### Preparation of Compound 73

Compound 73 was prepared in a manner similar to that described in Example 1. LC/MS (M+H⁺)=845.40.

### EXAMPLE 74

### Preparation of Compound 74

Compound 74 was prepared in a manner similar to that described in Example 9. LC/MS (M+H⁺)=834.10.

### EXAMPLE 75

### Preparation of Compound 75

Compound 75 was prepared in a manner similar to that described in Example 36. LC/MS (M+H⁺)=843.30.

### EXAMPLE 76

### Preparation of Compound 76

Compound 76 was prepared in a manner similar to that described in Example 36. LC/MS (M+H⁺)=843.30; ¹H NMR δ 7.91 (d, J=9.3 Hz, 1H), 7.82 (s, 1H), 7.75 (brs, 1H), 7.53-7.36 (m, 2H), 6.97 (d, J=8.1 Hz, 1H), 6.88 (s, 1H), 6.58-6.43 (m, 1H), 5.87-5.73 (m, 1H), 5.66 (brs, 1H), 5.35 (brs, 1H), 5.22 (d, J=17.1 Hz, 1H), 5.10 (d, J=10.2 Hz, 1H), 4.94 (brs, 1H), 4.57-4.38 (m, 2H), 4.28-4.03 (m, 2H), 3.94 (s, 3H), 3.40 (dd, J=8.1 Hz, J=8.1 Hz, 2H), 3.09-2.96 (m, 2H), 2.81 (s, 3H), 2.64-2.32 (m, 2H), 2.21-1.92 (m, 3H), 1.82-1.38 (m, 8H), 1.36-1.16 (m, 4H), 1.08-0.78 (m, 8H).

### EXAMPLE 77

### Preparation of Compound 77

Compound 77 was prepared in a manner similar to that described in Example 9. LC/MS (M+H⁺)=846.30.

### EXAMPLE 78

### Preparation of Compound 78

Compound 78 was prepared in a manner similar to that described in Example 9. LC/MS (M+H⁺)=846.30; ¹H NMR δ 7.95 (d, J=9.0 Hz, 1H), 7.61-7.48 (m, 3H), 7.37 (s, 1H), 7.01 (d, J=9.3 Hz, 1H), 6.96 (d, J=8.4 Hz, 1H), 6.86 (s, 1H), 5.87-5.73 (m, 2H), 5.30 (brs, 1H), 5.21 (d, J=17.4 Hz, 1H), 5.09 (d, J=10.5 Hz, 1H), 4.86 (brs, 1H), 4.58-4.43 (m, 2H), 4.30 (s, 4H), 4.18 (dd, J=8.7 Hz, J=8.7 Hz, 1H), 4.32-4.01 (m, 1H), 3.93 (s, 3H), 2.92-2.80 (m, 1H), 2.59 (dd, J=13.5 Hz, J=6.3 Hz, 1H), 2.44-2.32 (m, 1H), 2.21-2.02 (m, 2H), 1.95 (dd, J=6.0 Hz, J=6.0 Hz, 1H), 1.78-1.34 (m, 7H), 1.32-1.05 (m, 4H), 1.02-0.82 (m, 8H).

### EXAMPLE 79

### Preparation of Compound 79

Compound 79 was prepared in a manner similar to that described in Example 9. LC/MS (M+H⁺)=859.30.

### EXAMPLE 80

### Preparation of Compound 80

Compound 80 was prepared in a manner similar to that described in Example 9. LC/MS (M+H⁺)=859.30; ¹H NMR δ 7.90 (d, J=9.0 Hz, 1H), 755 (s, 1H), 7.53 (d, J=8.7 Hz, 1H), 7.45 (s, 1H), 7.37 (s, 1H), 6.97 (d, J=8.7 Hz, 1H), 6.84 (s, 1H), 6.70 (d, J=7.2 Hz, 1H), 5.87-5.69 (m, 2H), 5.29 (brs, 1H), 5.21 (d, J=17.4 Hz, 1H), 5.09 (d, J=10.5 Hz, 1H), 4.91 (brs, 1H), 4.56-4.39 (m, 2H), 4.30 (brs, 2H), 4.21 (dd, J=8.4 Hz, J=8.4 Hz, 1H), 4.12-3.96 (m, 1H), 3.93 (s, 3H), 3.33 (brs, 2H), 2.87 (s, 3H), 2.59-2.50 (m, 1H), 2.44-2.32 (m, 1H), 2.21-2.02 (m, 2H), 1.97 (dd, J=6.0 Hz, J=6.0 Hz, 1H), 1.79-1.36 (m, 8H), 1.32-1.06 (m, 4H), 1.04-0.84 (m, 8H).

### EXAMPLE 81

### Inhibition of NS3/4A Protein

### Protein Expression and Purification

A plasmid containing N-terminal His₆-tagged-NS4A₍₂₁₋₃₂₎-GSGS-NS3₍₃₋₁₈₁₎ was transformed into E. coli strain BL21(DE3)pLysS (Novagen) for protein over-expression. Single colony of transformed BL21 (DE3)pLysS was cultured in 200 mL of Lauria-Bertani (LB) medium with Kanamycin and Chloramphenicol at 37° C. overnight. The bacterial culture was transferred into 6 L LB medium (Difco) containing antibiotics and incubated with shaking at 22° C. After the absorbance at 600 nm reached 0.6, the culture was induced with 1 mM isopropyl-1-thio-β-D-galactopyranoside (IPTG) at 22° C. for 5 hours. The culture was subsequently harvested by centrifugation (6,000×g for 15 minutes at 4° C.). Cell pellets were resuspended in 150 mL buffer A (50 mM HEPES, pH 7.4, 0.3 M NaCl, 0.1% (w/v) CHAPS, 10 mM imidazol, 10% (v/v) glycerol). After four passes through a Microfluidizer operated at 30 psi disrupted the mixture, the cell debris was removed by centrifugation (58,250×g for 30 minutes at 4° C.). The cell lysate containing His₆-tagged proteins was applied at 3 mL/min to a 25 ml Ni-NTA (Qiagen) column in the presence of 10 mM imidazole using a GradiFrac system (Pharmacia). The column was washed with 10 column volumes of the lysis buffer. The bound NS4A₍₂₁₋₃₂₎-GSGS-NS3₍₃₋₁₈₁₎ was eluted with 8 column volumes of buffer A supplemented with 300 mM imidazole. The pooled fractions were further purified by Q-Sepharose column equilibrated in buffer B (50 mM HEPES, pH 7.4, 0.1% (w/v) CHAPS, 10% (v/v) glycerol, 5 mM dithiothreitol (DTT), and 1 M NaCl). The eluant containing NS4A₍₂₁₋₃₂₎-GSGS-NS3₍₃₋₁₈₁₎ was collected. Fractions containing NS4A₍₂₁₋₃₂₎-GSGS-NS3₍₃₋₁₈₁₎ were collected and further purified by size-exclusion chromatography using Sephacryl-75 columns (16×100 cm, Pharmacia) at a flow rate of 0.5 mL/min. Columns were pre-equilibrated in buffer C (50 HEPES, pH 7.4, 0.1% (w/v) CHAPS, 5 mM DTT, 10% (v/v) glycerol). The purified protein was frozen and stored at −80° C. before use.

### Inhibition Assay Protocol

The HPLC Microbore assay for separation of HCV protease substrate and products was used. The substrate used in the assay was Ac-Asp-Glu-Asp(EDANS)-Glu-Glu-Abu-ψ-[COOAla]-Ser-Lys(DABCYL)-NH₂ (RET S1, ANASPEC). The buffer used in the assay included 50 mM Tris buffer, pH 7.4, 100 mM NaCl, 20% glycerol, and 0.012% CHAPS.

A stock aqueous solution of 10 mM substrate RET S1 was prepared and stored in aliquots at −80° C. before use. DTT, RET S1, and a test compound were dissolved in the buffer (the final volume: 80 μL), and then the solution was added to a well of a 96-well plate. Reaction was initiated by addition of 20 μL of 10 nM NS3/4A protease in the buffer to form a 100 μL assay solution, which contained 50 mM Tris, pH 7.4, 100 mM NaCl, 20% glycerol, 0.012% CHAPS, 10 mM DTT, 5 μM substrate RET S1, and 10 μM the test compound. The final concentration of NS3/4A protease was 2 nM, which was lower than the Km of substrate RET S1.

The assay solution was incubated for 30 minutes at 30° C. The reaction was then terminated by addition of 100 μL of 1% TFA. 200 μL aliquot was transferred to each well of Agilent 96-well plates for the next step.

Separation of Product from Substrate

The reaction products were analyzed using reverse phase HPLC described below. The HPLC system includes: Agilent 1100, Degasser G1379A, Binary pump G1312A, Autosampler G1367A, Column thermostated chamber G1316A, Diode array detector G1315B, Column: Agilent, ZORBAX Eclipse XDB-C18, 4.6 mm, 5 μm, P/N 993967-902, Column thermostat: room temperature, Injection volume: 100 μL; Solvent A=HPLC grade water+0.09% TFA, Solvent B=HPLC grade acetonitrile+0.09% TFA. Total HPLC running time was 7.6 minutes with a linear gradient of acetonitrile from 25 to 50% B within 4 minutes, 50% B for 30 seconds, and a gradient from 50 to 25% B within 30 seconds. The column was re-equilibrated with 25% B for 2.6 minutes before the next sample was injected. The IC₅₀ value (the concentration at which 50% inhibition of NS3/4A was achieved) was calculated for each test compound based on the HPLC results.

Compounds 1-80 were tested in the above inhibition assay. The results showed that 59 compounds exhibited IC₅₀ values lower than 1 μM, 14 compounds exhibited IC₅₀ values in the range of I-10 μM, and 7 compounds exhibited IC₅₀ values higher than 10 μM. Some of the test compounds surprisingly exhibited IC₅₀ values even lower than 50 nM.

### EXAMPLE 82

### HCV Replicon Cell Assay Protocol

HCV replicon Cells were maintained in a media (media A), which contains DMEM including 10% fetal bovine serum (FBS), 1.0 mg/ml G418, and appropriate supplements.

On day 1, the replicon cell monolayer was treated with a trypsin/EDTA mixture, removed, and diluted with media A to give a final concentration of 48,000 cells/ml. The solution (1 ml) was added to each well of a 24-well tissue culture plate, and cultured overnight in a tissue culture incubator at 37° C. with 5% CO₂.

On day 2, each test compound (in DMSO) was diluted with DMEM containing 10% FBS and appropriate supplements to provide a series of sample solutions having different concentrations. The final concentration of DMSO was maintained at 0.2% throughout the dilution series.

The media was removed from wells containing the replicon cell monolayer, and then the sample solutions were added. DMEM containing 10% FBS and appropriate supplements, but no compound, were added to other wells as compound-free controls.

The cells were incubated with a compound or 0.2% DMSO in a media the same as media A described above except G418 is absent for 72 hours in a tissue culture incubator with 5% CO₂ at 37° C. The media was removed, and the replicon cell monolayer was washed once with PBS and extracted total cellular RNA. RNA extraction reagents (e.g., reagents from RNeasy kits or TRIZOL reagents) were added to the cells immediately to avoid degradation of RNA. Total RNA was extracted according to the manufacturer's instructions with modification to improve extraction efficiency and consistency. Finally, total cellular RNA, including HCV replicon RNA, was eluted and stored at −80° C. before further processing.

A TaqMan® real-time RT-PCR quantification assay was set up with two sets of specific primers and probe. One was for HCV and the other was for ACTB (beta-actin). The total RNA extractants from the treated HCV replicon cells were added to the PCR reactions for quantification of both HCV and ACTB RNA in the same PCR well. Experimental failure was flagged and rejected based on the level of ACTB RNA in each well. The level of HCV RNA in each well was calculated according to a standard curve run in the same PCR plate. The percentage of inhibition of HCV RNA level by the compound treatment was calculated using the DMSO or no-compound control as 0% of inhibition. EC₅₀ (the concentration at which 50% inhibition of HCV RNA level was achieved) was calculated from the titration curve of any given compound.

Compounds 1-80 were tested in the HCV replicon cell assay. The results showed that all test compounds exhibited inhibitory effect against the HCV RNA level. Some test compounds surprisingly had very low EC₅₀ values. For example, 23 compounds had EC₅₀ values lower than 50 nM, and 25 compounds had EC₅₀ values between 50-500 nM, and 32 compounds had EC₅₀ values higher than 500 nM. Some of the test compounds surprisingly exhibited EC₅₀ values even lower than 50 nM.

### OTHER EMBODIMENTS

All of the features disclosed in this specification may be combined in any combination. An alternative feature serving the same, equivalent, or similar purpose may replace each feature disclosed in this specification. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 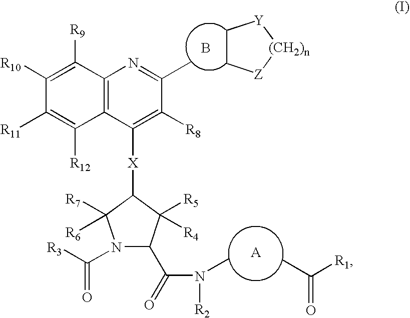
wherein
A is C₃-C₅ cycloalkylene, C₃-C₅ cycloalkenylene, or C₇-C₂₀ alkylarylene;
B is aryl or heteroaryl;
X is O, OCH₂, CH₂O, OC(O), CO(O), C(O)NH, or NHC(O);
each of Y and Z, independently, is N(Rₐ₁), O, or CH₂; in which Rₐ₁ is H, C₁-C₁₀ alkyl, C₃-C₂₀ cycloalkyl, C₁-C₂₀ heterocycloalkyl, aryl, or heteroaryl;
n is 1 or 2;
R₁ is O(R_{b1}), NR_{b1}R_{b2}, NH—O(R_{b1}), NH—C(O)—R_{b1}, NH—C(O)—NR_{b1}R_{b2}, NH—NH—C(O)—R_{b1}, NH—C(O)—NH—S(O)₂—R_{b1}, NH—C(O)—COOR_{b1}, C(O)—NR_{b1}R_{b2}, NH—(R_{b3})—(R_{b4})—S(O)₂—R_{b1}, NH—(R_{b3})—(R_{b4})—S(O)₂—NR_{b1}R_{b2}, or NH—(R_{b3})—(R_{b4})—COO—R_{b1}; in which each of R_{b1} and R_{b2}, independently, is H, C₁-C₁₀ alkyl, C₃-C₂₀ cycloalkyl, C₁-C₂₀ heterocycloalkyl, aryl, or heteroaryl, and each of R_{b3} and R_{b4}, independently, is C₁-C₁₀ alkylene, C₃-C₂₀ cycloalkylene, C₁-C₂₀ heterocycloalkylene, arylene, heteroarylene, or deleted; and
each of R₂, R₃, R₄, R₅, R₆, R₇, R₈, R₉, R₁₀, R₁₁, and R₁₂, independently is H, halo, OR_{c1}, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl, in which R_{c1} is H, C₁-C₁₀ alkyl, C₃-C₂₀ cycloalkyl, C₁-C₂₀ heterocycloalkyl, aryl, or heteroaryl.

2. The compound of claim 1, wherein X is O.

3. The compound of claim 2, wherein B is phenyl, pyridyl, or thiazole.

4. The compound of claim 3, wherein A is 1,1-cyclobutylene, 1,1-cyclopentylene, 1,1-cyclopropylene optional substituted with C₂-C₁₀ alkenyl, 1,1-cyclopentenylene optionally substituted with C₂-C₁₀ alkenyl, or 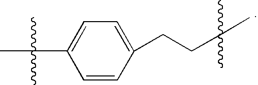
optionally substituted with C₁-C₁₀ alkyl or hydroxyl.

5. The compound of claim 4, wherein R₁ is O(R_{b1}), NH—C(O)—NR_{b1}R_{b2}, NH—NH—C(O)—R_{b1}, NH—C(O)—NH—S(O)₂—R_{b1}, NH—C(O)—COOR_{b1}, NH—(R_{b3})—(R_{b4})—S(O)₂—R_{b1}, NH—(R_{b3})—(R_{b4})—S(O)₂—NR_{b1}R_{b2}, or NH—(R_{b3})—(R_{b4})—COO—R_{b1}.

6. The compound of claim 5, wherein R₁ is OH, NH—S(O)₂—C₁-C₁₀ alkyl, NH—S(O)₂-phenyl, NH—S(O)₂-cyclopropyl, NH—S(O)₂—NH—C₁-C₁₀ alkyl, NH—S(O)₂—NH-phenyl, NH—S(O)₂—NH-cyclopropyl, NH—C(O)—NH-phenyl, NH—NH—C(O)-thienyl, NH—C(O)—NH—S(O)₂-(4-methylphenyl), NH—C(O)—NH-thienyl, NH—C(O)—NH—S(O)₂-phenyl, NH—CH(OH)—COO-ethyl, or NH—C(O)—COO-ethyl.

7. The compound of claim 6, wherein R₃ is aryl optionally fused with C₁-C₂₀ heterocycloalkyl, or C₁-C₁₀ alkyl optional substituted with NH—COOR or C₁-C₂₀ heterocycloalkyl, in which R is H, C₁-C₁₀ alkyl, C₃-C₂₀ cycloalkyl, C₁-C₂₀ heterocycloalkyl, aryl, or heteroaryl.

8. The compound of claim 7, wherein R₃ is benzo[1,3]dioxolyl, or isobutyl substituted with NH—COO-t-butyl, NH—COO-cyclopentyl, 3-cyclopentylimidazolidinonyl, or 3-t-butylimidazolidinonyl.

9. The compound of claim 1, wherein the compound is one of compounds 1-15 and 24-80.

10. The compound of claim 1, wherein X is OCH₂, CH₂O, OC(O), CO(O), C(O)NH, or NHC(O).

11. The compound of claim 10, wherein B is phenyl.

12. The compound of claim 11, wherein A is 1,1-cyclobutylene or 1,1-cyclopropylene optional substituted with C₂-C₁₀ alkenyl.

13. The compound of claim 12, wherein R₁ is O(R_{b1}) or NH—(R_{b3})—(R_{b4})—S(O)₂—R_{b1}.

14. The compound of claim 13, wherein R₁ is OH, NH—S(O)₂-phenyl, or NH—S(O)₂-cyclopropyl.

15. The compound of claim 14, wherein R₃ is C₁-C₁₀ alkyl optional substituted with NH—COOR, in which R is H, C₁-C₁₀ alkyl, C₃-C₂₀ cycloalkyl, C₁-C₂₀ heterocycloalkyl, aryl, or heteroaryl.

16. The compound of claim 15, wherein R₃ is isobutyl substituted with NH—COO-t-butyl or NH—COO-cyclopentyl.

17. The compound of claim 1, wherein the compound is one of compounds 16-23.

18. The compound of claim 1, wherein B is phenyl, pyridinyl, or thiazole; and n is 1 or 2.

19. The compound of claim 18, wherein each of Y and Z is O; each of Y and Z is CH; or Y is NH or NC₁-C₁₀ alkyl and Z is CH or O.

20. The compound of claim 1, wherein R₁ is NH—S(O)₂-cyclopropyl.

21. A method for treating an infection with hepatitis C virus, comprising administering to a subject in need thereof an effective amount of a compound of claim 1.

22. The method of claim 18, wherein X is O or OCH₂; B is phenyl, pyridyl, or thiazole; and R₁ is OH, NH—S(O)₂—C₁-C₁₀ alkyl, NH—S(O)₂-phenyl, NH—S(O)₂-cyclopropyl, NH—S(O)₂—NH—C₁-C₁₀ alkyl, NH—S(O)₂—NH-phenyl, NH—S(O)₂—NH-cyclopropyl, NH—C(O)—NH-phenyl, NH—NH—C(O)-thienyl, NH—C(O)—NH—S(O)₂-(4-methylphenyl), NH—C(O)—NH-thienyl, NH—C(O)—NH—S(O)₂-phenyl, NH—CH(OH)—COO-ethyl, or NH—C(O)—COO-ethyl.

23. The method of claim 21, wherein the compound is one of compounds 1-80.

24. A pharmaceutical composition, comprising a pharmaceutically acceptable carrier and a compound of claim 1.

25. The composition of claim 24, wherein the compound is one of compounds 1-80. treat hepatitis C virus infection. [chemical structure]
